(19) United States
(12) Patent Application Publication
(10) Pub. No.: US 2024/0109852 A1
(43) Pub. Date: Apr. 4, 2024

(54) Ferroptosis-HDAC Inhibitor Hybrid Anticancer Agents

(71) Applicant: The University of Toledo, Toledo, OH (US)

(72) Inventors: Viranga Tillekeratne, Toledo, OH (US); Endri Karaj, Toledo, OH (US); Shaimaa Sindi, Toledo, OH (US); William Taylor, Toledo, OH (US)

(73) Assignee: The University of Toledo, Toledo, OH (US)

(21) Appl. No.: 18/238,687

(22) Filed: Aug. 28, 2023

Related U.S. Application Data
(60) Provisional application No. 63/401,893, filed on Aug. 29, 2022.

(51) Int. Cl. C07D 277/32; A61P 35/00

(52) U.S. Cl. CPC C07D 277/32; A61P 35/00

(57) ABSTRACT

Hybrid molecules having pharmacophores of a ferrotoptosis inducer and an HDAC inhibitor, as well as ferroptosis inducers and HDAC inhibitors, and methods of making and using the same, are described.

### RELATED APPLICATIONS

This application claims priority to U.S. Provisional Application No. 63/401,893 filed under 35 U.S.C. § 111(b) on Aug. 29, 2022, the disclosure of which is incorporated herein by reference in its entirety.

### STATEMENT REGARDING FEDERALLY SPONSORED RESEARCH

This invention was made with government support under Grant Number 1R15CA213185-01A1 awarded by the National Institutes of Health. The government has certain rights in this invention.

### BACKGROUND

Chemotherapy remains one of the major approaches in cancer treatment with small molecules still dominating the market. These small organic compounds target diverse pathways, causing cell cycle arrest and death of cancer cells. Due to genetic instability, tumors become more heterogeneous during the progression of the disease, leading to cells with distinct molecular signatures. When this diverse population is subjected to chemotherapeutic agents, drug resistance can emerge due to chemotherapy acting as an evolutionary pressure to select for cells that can grow in the presence of the drug. Indeed, clinically, there is a negative correlation between the diversity of the tumor and therapeutic outcome. Despite being extensively studied, drug resistance remains a major impediment in cancer treatment. Combinatorial treatment approaches are adopted to decrease the chances of drug resistance and increase the chances of tumor eradication. In this approach, chemotherapy can be combined with radiotherapy or surgery, or in the alternative, a combination of two or more drugs can be used to target different cell survival pathways. More than 370 drug combinations have been approved by the food and drug administration FDA.

Combination therapy was first conceptualized by showing the benefits of combining methotrexate, 6-mercaptopurine, vincristine, and prednisone (POMP regimen) to treat pediatric patients with acute lymphocytic leukemia. Since then, numerous drug combination regimens with synergistic or additive effects have been established, allowing reduced dosage requirements and, therefore, fewer side effects. Combining drugs is a common practice due to the advantages of drug combinations over monotherapy. However, the combined drugs remain different molecular entities with different pharmacological properties. Differences in biodistribution at both the cellular level and the organism level can hinder the successful administration of drug combinations. Thus, combinatorial therapy works best when drugs demonstrate similar pharmacokinetic profiles, which limits the available options.

An alternative to drug combinations is the rational design of multi-targeted hybrid molecules which ensure homogenous spatiotemporal biodistribution of the individual active entities. In this approach, pharmacophoric features from the drugs that are to be combined are incorporated into a single scaffold which maintains the pharmacodynamic effects of the individual drugs. The hybrid molecule ensures that the required activities are uniformly distributed in space and time. However, a potential drawback of hybrid molecules is the limitation of a fixed equimolar ratio of the two components. In addition, the design of hybrid molecules is extremely challenging because many pharmacophores do not tolerate significant structural changes.

One class of drugs useful for the multi-targeted hybrid molecules is histone deacetylase inhibitors (HDACis). HDACis act as epigenetic regulators by inhibiting histone deacetylases. This class of enzymes, in combination with histone acetyl transferases (HATs), control chromatin remodeling by regulating the acetylation levels of histones. HDACs also deacetylate numerous non-histone proteins contributing to the effects of HDAC inhibitors on a plethora of diverse cellular functions such as proliferation, cell death, metastasis, autophagy, metabolism, and ciliary expression. Based on phylogenetic comparison with yeast homologues, HDAC proteins are classified into four classes. Class I includes HDAC-1, HDAC-2, HDAC-3, and HDAC-8. Class 1 HDAC proteins use Zn²⁺ as a co-factor and mainly localize in the nucleus with strong deacetylase activity towards histones. Class II HDACs use Zn²⁺ as a co-factor as well, and are further divided into two subclasses, class IIa, which includes HDAC-4, HDAC-5, HDAC-7, and HDAC-9, and class IIb, which includes HDAC-6 and HDAC-10. Subclass IIa HDACs are found in both the nucleus and the cytoplasm and control the activities of several non-histone proteins like myocyte enhancer factor-2 (MEF2), while subclass IIb HDACs are found mainly in the cytoplasm with deacetylase activities against several interesting targets like tubulin deacetylation by HDAC-6 which regulates microtubule stability. Class III HDACs are NADtdependent enzymes that do not use Zn²⁺ as a co-factor and are referred to as sirtuins (SIRT 1-7). Class IV contains only HDAC-11, which uses Zn²⁺ as a co-factor and has similarities with both class I and class II HDACs. Very little is known about the biochemical function of HDAC-11, though it is believed to have a role in immune activation and tumorigenesis.

Elevated levels of several HDAC isoforms are associated with tumor survival and progression. For example, prostate cancers have elevated levels of HDAC1, while gastric, colorectal carcinomas, cervical, and endometrial cancers all overexpress HDAC2, when compared to the corresponding normal cells. These observations make HDACis possible drug candidates. Four HDACis have already gained FDA approval and are important clinically used drugs. However, the use of HDACis is currently limited to hematological malignancies, and clinical trials are underway to use them for solid tumors in combination with other drugs.

A typical HDACi pharmacophore consists of a zinc-binding group (ZBG), a short planar aliphatic or aromatic linker, and a cap group (FIG. 1B). The ZBG and the linker are important for HDAC inhibition as they are necessary to access and bind to the zinc ion in the internal cavity of the enzyme (FIG. 2A). The amino acids in these regions are highly conserved among the different HDAC isoforms. The cap group binds to a less conserved area around the rim at the entrance to the active site (FIG. 2A). HDACis have the ability to tolerate cap groups of diverse structure (preferably aromatic systems) without affecting the HDAC inhibitory capabilities significantly. With large cap groups, isoform selectivity may be attained, as exemplified by the SAHA (Vorinostat) analogue tubacin. At the cellular level, the net effect of HDAC inhibition can be summarized as induction of apoptosis. In fact, most hybrid molecules to date (including HDACi hybrids) combine two pharmacophores that target different cellular pathways, but both leading to apoptotic death such as hybrids of HDACs with cyclin-dependent kinases (CDK), topoisomerase (topo), bromodomain (BDR) inhibitors, and DNA cross-linkers (some examples of which are shown in FIGS. 1C-1E). There is a need in the art for new and improved hybrid molecules with anticancer activity.

### SUMMARY

Provided is a composition comprising a hybrid molecule having both a ferroptotic pharmacophore and a histone deacetylase (HDAC) inhibitor pharmacophore.

In certain embodiments, the composition further comprises a pharmaceutically acceptable carrier, diluent, or adjuvant.

In certain embodiments, the ferroptotic pharmacophore comprises a terminal alkyne attached to a thiazole. In certain embodiments, the HDAC inhibitor pharmacophore comprises a hydroxamic acid metal-binding group.

In certain embodiments, the hybrid molecule comprises a zinc-binding group (ZBG) connected to a linker, and a cap group connected to the linker. In particular embodiments, the ZBG comprises a hydroxamate. In particular embodiments, the linker comprises an aliphatic chain. In particular embodiments, the ZBG comprises a hydroxxamate, and the linker comprises an aliphatic chain. In particular embodiments, the cap group comprises a terminal alkyne attached to a thiazole. In particular embodiments, the cap group comprises a terminal alkyne at the 2-position of a thiazole ring. In particular embodiments, the cap group further comprises an amide.

In certain embodiments, the ZBG comprises a hydroxamate, the linker comprises an aliphatic chain, and the hydroxamate comprises a terminal alkyne attached to a thiazole. In particular embodiments, the cap group further comprises an amide.

In certain embodiments, the hybrid molecule has Formula A:

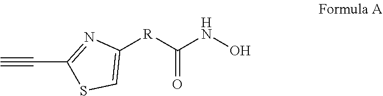

wherein R is alkyl, alkenyl, amidoalkyl, amidoalkenyl, arylalkyl, arylalkenyl, or amidoarylalkenyl. Also provided are salts, stereoisomers, racemates, solvates, hydrates, and polymorphs of Formula A.

In certain embodiments, R is (C1-C6)alkyl or (C1-C6)alkenyl. In certain embodiments, R is ethyl or ethenyl. In certain embodiments, R includes an aliphatic chain of from 4 to 6 carbons. In particular embodiments, R further includes either an alkenyl group or an amido group.

In certain embodiments, the hybrid molecule is HY-1:

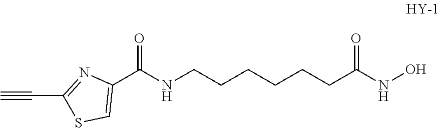 [SMILES: C#Cc1nc(C(=O)NCCCCCCC(=O)NO)cs1]

In certain embodiments, the hybrid molecule is HY-2:

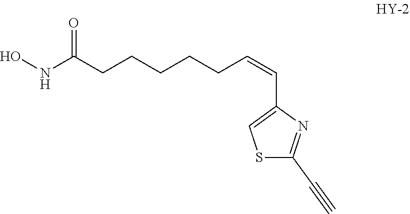 [SMILES: C#Cc1nc(/C=C\CCCCCC(=O)NO)cs1]

In certain embodiments, the hybrid molecule is HY-3:

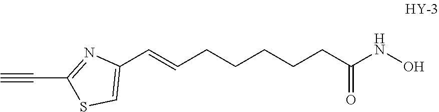 [SMILES: C#Cc1nc(/C=C/CCCCCC(=O)NO)cs1]

In certain embodiments, the hybrid molecule is HY-4:

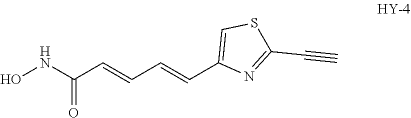

In certain embodiments, the hybrid molecule is HY-5:

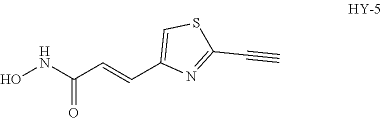

Further provided is a method of killing cancer cells, the method comprising contacting cancer cells with an effective amount of a composition comprising a hybrid molecule described herein, and killing the cancer cells.

In certain embodiments, the cancer is triple negative breast cancer.

In certain embodiments, the cancer is renal cancer, leukemia, colon cancer, ovarian cancer, breast cancer, lung cancer, melanoma, or CNS cancer, and the composition comprises HY-1.

In certain embodiments, the cancer is renal cancer, leukemia, ovarian cancer, breast cancer, lung cancer, melanoma, or CNS cancer, and the composition comprises HY-2.

In certain embodiments, the cancer is renal cancer, leukemia, ovarian cancer, lung cancer, melanoma, or CNS cancer, and the composition comprises HY-3.

In certain embodiments, the cancer is renal cancer, leukemia, ovarian cancer, breast cancer, lung cancer, melanoma, or CNS cancer, and the composition comprises HY-5.

Further provided is a method of treating a cancer, the method comprising administering an effective amount of a composition comprising a hybrid molecule described herein to a subject having a cancer, and treating the cancer.

Further provided is a composition comprising a ferroptotic inducer having Formula B:

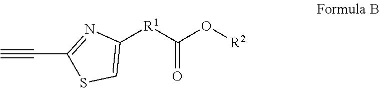

where R¹ is alkyl, alkenyl, amidoalkyl, amidoalkenyl, arylalkyl, arylalkenyl, or amidoarylalkenyl; and R² is alkyl. Also provided are salts, stereoisomers, racemates, solvates, hydrates, and polymorphs of Formula B. In certain embodiments, the composition further comprises a pharmaceutically acceptable carrier, diluent, or adjuvant.

In certain embodiments, R² is methyl or ethyl. In certain embodiments, R¹ is (C1-C6)alkyl or (C1-C6)alkenyl. In certain embodiments, R¹ is ethyl or ethenyl. In certain embodiments, R¹ includes an aliphatic chain of from 4 to 6 carbons. In particular embodiments, R¹ further includes either an alkenyl group or an amido group.

In certain embodiments, the ferroptotic inducer is FC-1:

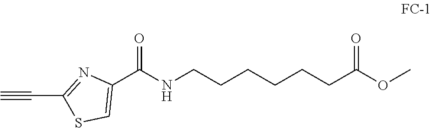 [SMILES: C#Cc1nc(C(=O)NCCCCCCC(=O)OC)cs1]

In certain embodiments, the ferroptotic inducer is FC-2:

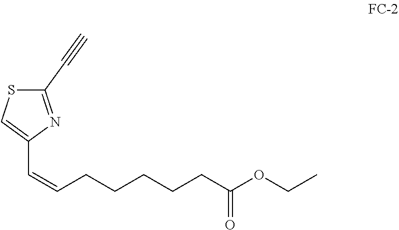 [SMILES: C#Cc1nc(/C=C\CCCCCC(=O)OCC)cs1]

In certain embodiments, the ferroptotic inducer is FC-3:

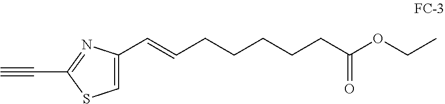 [SMILES: C#Cc1nc(/C=C/CCCCCC(=O)OCC)cs1]

In certain embodiments, the ferroptotic inducer is FC-4:

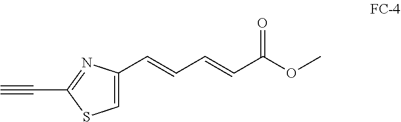

In certain embodiments, the ferroptotic inducer is FC-5:

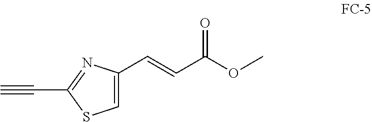

Further provided is a method of killing cancer cells, the method comprising contacting cancer cells with an effective amount of a composition comprising a ferroptotic inducer described herein, and killing the cancer cells.

In certain embodiments, the cancer cells are neuroblastoma cells. In certain embodiments, cancer cells are neuroblastoma cells, and the ferroptotic inducer is FC-1.

Further provided is a method of treating a cancer, the method comprising administering to a subject having cancer an effective amount of a composition comprising a ferroptotic inducer described herein, and treating the cancer.

Further provided is a method of inducing ferroptosis in a cell, the method comprising contacting a cell with an effective amount of a composition comprising a ferroptotic inducer described herein, and inducing ferroptosis in the cell

Further provided is a composition comprising a histone deacetylase (HDAC) inhibitor having Formula C:

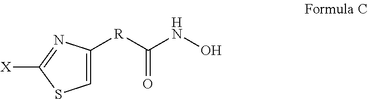

where X is a halide, and R is an alkyl, alkenyl, alkynyl, amidoalkyl, amidoalkenyl, arylalkyl, arylalkenyl, or amidoarylalkenyl group. Also provided are salts, stereoisomers, racemates, solvates, hydrates, and polymorphs of Formula C. In certain embodiments, the composition further comprises a pharmaceutically acceptable carrier, diluent, or adjuvant.

In certain embodiments, X is Br. In certain embodiments, X is Br, and R includes an aliphatic chain of from 4 to 6 carbons. In certain embodiments, X is Br, and R includes an alkenyl group.

In certain embodiments, wherein the HDAC inhibitor is HC-1:

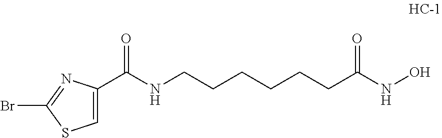 [SMILES: O=C(CCCCCCNC(=O)c1csc(Br)n1)NO]

In certain embodiments, wherein the HDAC inhibitor is HC-2:

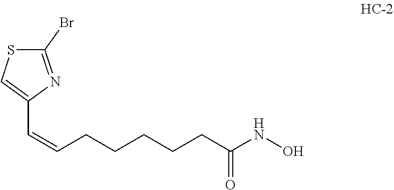 [SMILES: O=C(CCCCC/C=C\c1csc(Br)n1)NO]

In certain embodiments, wherein the HDAC inhibitor is HC-3:

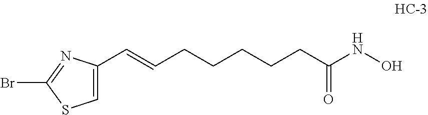 [SMILES: O=C(CCCCC/C=C/c1csc(Br)n1)NO]

In certain embodiments, wherein the HDAC inhibitor is HC-4:

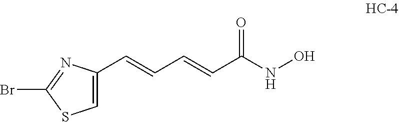 [SMILES: O=C(/C=C/C=C/c1csc(Br)n1)NO]

In certain embodiments, wherein the HDAC inhibitor is HC-5:

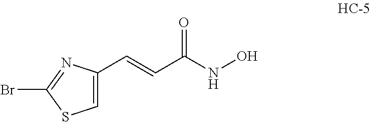 [SMILES: O=C(/C=C/c1csc(Br)n1)NO]

Further provided is a method of killing cancer cells, the method comprising contacting cancer cells with an effective amount of a composition comprising a HDAC inhibitor described herein, and killing the cancer cells. In certain embodiments, the cancer cells are leukemia cells.

Further provided is method of treating a cancer, the method comprising administering to a subject having cancer an effective amount of a composition comprising an HDAC inhibitor described herein, and treating the cancer.

Further provided is a method of inhibiting histone deacetylase (HDAC) in a subject, the method comprising administering to the subject an effective amount of a composition comprising an HDAC inhibitor described herein, and inhibiting HDAC in the subject.

Further provided is a method of making an anticancer agent, the method comprising combining a ferroptosis inducing pharmacophore with a HDAC inhibitor pharmacophore to produce an anticancer agent comprising a ferroptotic inducing HDAC inhibitor hybrid molecule.

Further provided is a method of killing cancer cells, the method comprising contacting cancer cells with an effective amount of a composition comprising Formula A, Formula B, or Formula C, and killing the cancer cells:

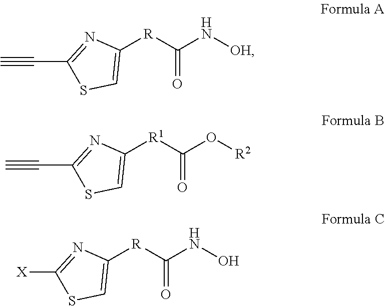

wherein R is alkyl, alkenyl, amidoalkyl, amidoalkenyl, arylalkyl, arylalkenyl, or amidoarylalkenyl; X is a halide; R¹ is alkyl, alkenyl, amidoalkyl, amidoalkenyl, arylalkyl, arylalkenyl, or amidoarylalkenyl; and R² is alkyl. In certain embodiments, the cancer is triple negative breast cancer. In certain embodiments, the cancer is renal cancer, leukemia, colon cancer, ovarian cancer, breast cancer, lung cancer, melanoma, or CNS cancer.

Further provided is a method of treating a cancer, the method comprising administering to a subject having cancer an effective amount of a composition comprising Formula A or Formula C, and treating the cancer:

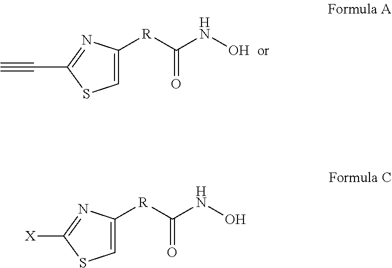

wherein R is alkyl, alkenyl, amidoalkyl, amidoalkenyl, arylalkyl, arylalkenyl, or amidoarylalkenyl; and X is a halide.

Further provided is a method of inhibiting histone deacetylase (HDAC) in a subject, the method comprising administering to the subject an effective amount of a composition comprising Formula A or Formula C, and inhibiting HDAC in the subject:

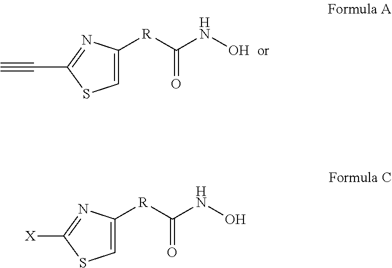

wherein R is alkyl, alkenyl, amidoalkyl, amidoalkenyl, arylalkyl, arylalkenyl, or amidoarylalkenyl; and X is a halide.

Further provided is a method of making an anticancer agent, the method comprising combining a ferroptosis inducing pharmacophore with a HDAC inhibitor pharmacophore to produce an anticancer agent comprising a ferroptotic inducing HDAC inhibitor hybrid molecule.

### BRIEF DESCRIPTION OF THE DRAWINGS

The patent or application file contains at least one drawings executed in color. Copies of this patent or patent application publication with color drawing(s) will be provided by the Office upon request and payment of the necessary fee.

FIGS. 1A-1E: FIG. 1A shows an illustration of some example molecular tools for designing HDAC hybrids composed of ZBG, linkers, and cap groups. Among these, the cap group, which typically prefers to have some aromatic components, tolerates diverse groups without loss of activity. FIGS. 1B-1E show examples of HDAC inhibitor hybrids which utilize some of the tools illustrated in FIG. 1A.

FIGS. 2A-2E: 3D representation of the active site of HDAC-8 PDB id: 1W22 (FIG. 2A). A typical HDAC active site includes a zinc-binding cavity, which is connected to an outside rim through a narrow tunnel. This active site structure is composed of a zinc binding group which chelates with zinc, a linker that occupies the narrow tunnel, and a cap group which covers the outside rim area. FIG. 2B shows a pose analysis of the HY-1 molecule and SAHA, which shows that the hybrid molecule HY-1 positions its ZBG and linker in an identical way to SAHA, while the cap group has a slightly different orientation (vide infra for docking studies). FIGS. 2C-2D show representations of binding interactions for HY-1 (FIG. 2C) and SAHA (FIG. 2D). In both, the ZBG demonstrates bi-dentate chelation with a zinc ion. In addition, hydrogen bonding interactions with HIS142 and TYR306 further facilitate the positioning of the ZBG in the zinc-containing cavity. On HDAC-8, the cap group of HY-1 is positioned towards PHE207, while in SAHA, it is positioned towards the loop containing PRO35-ILE34-Lys33. FIG. 2E shows the rational design for the synthesis of the amide and olefinic series. Combination of the simplified CETZOLE-1 analog (1) and SAHA analog (2) provides the hybrid molecule HY-1. Combination of CETZOLE-1 analog (3) with SAHA analog (4) by Wittig reaction provides the hybrid molecules HY-(2-5).

FIGS. 3A-3D: Designed library of analogs including negative controls NC-(1-5) (FIG. 3A), ferroptosis inducers FC-(1-5) (FIG. 3B), HDACis HC-(1-5) (FIG. 3C), and hybrid molecules HY-(1-5) (FIG. 3D).

FIG. 4: Scheme 1, showing the synthesis of amide analogs. This synthetic route allows the access of amide analogs NC-1, FC-1, HC-1, and HY-1.

FIG. 5: Scheme 2, showing the synthesis of the olefinic analogs. This synthesis generates a mixture of isomers (and the corresponding N/F/HC-2-M and HY-2-M analogs), as well as the pure Z-isomers (and the corresponding N/F/HC-2 and HY-2 analogs). The pure E-analog was not obtained through this process.

FIG. 6: Scheme 3, depicting the synthesis of analogs containing a doubly unsaturated linker.

FIG. 7: Scheme 4, depicting the synthesis of the short-linker analogs.

FIG. 8: Scheme 5, depicting the synthesis of the pure E-analogs. The nature of the counter ion of the base used for the transformation of (23) to (24) dictates the resulting analogs. Use of Na⁺ provides E/Z mixtures, which allow the separation of some pure Z-product (NC-2). The use of Li⁺ provided E-enriched mixtures, which allowed for the partial separation of pure E-product.

FIG. 9: Cell survival rates of NCI-522 and HCT-116 cells after treatment with HY-1, CETZOLE-1, and SAHA (2.5 mM). HY-1 combines ferroptosis and HDACi, which leads to enhanced sensitivity of the NCI-H522 cell line over the parent molecules. HCT-116 cells are more resistant to ferroptosis; thus, HY-1 has significant effect over CETZOLE-1, but comparable to the effect with SAHA. Data are mean±SD. (n=3) and expressed as -fold decrease relative to the value for the control group (DMSO). Statistical analysis utilizing one-way ANOVA test *P<0.05, **P<0.01, ***P<0.001.

FIGS. 10A-10D: Cell survival assay of NCI-H522 cells with CETZOLE-1, SAHA, equimolar mixture of CETZOLE-1+SAHA; FC-1, HC-1, equimolar mixture of FC-1+HC-1, HY-1; FC-2, HC-2, equimolar mixture of FC-2+HC-2, and HY-2, at concentrations of 1 mM, 2 mM, and 5 mM. FIG. 10A shows equimolar combination of SAHA and CETZOLE-1 led to significant enhancement of cytotoxicity at a concentration of 2 mM when compared to the corresponding SAHA treatment. At lower (1 mM) or higher (5 mM) concentrations, no significant differences were observed. FIG. 10B shows that for FC-1, and HC-1, equimolar combinations behaved exactly like a combination of SAHA and CETZOLE-1. Enhanced cytotoxicity of the equimolar mix was observed only at 2 mM, when compared to the corresponding HC-1 treatments. However, HY-1, being highly cytotoxic, showed enhanced effects even at 1 mM when compared to HC-1 or the equimolar mix. At 2 mM and 5 mM, HY-1 behaved like the equimolar mix. FIG. 10C shows that for FC-2 and HC-2, a high concentration of 5 mM was required to observe the enhanced effects. In contrast, HY-2 demonstrated enhanced cytotoxicity even at 2 mM. FIG. 10D shows the enhanced cytotoxicity of HY-1 and equimolar mix of SAHA and CETZOLE was not limited to the NCI-H522 cell line. MDA-MB-231 cells were more sensitive to HY-1 (2.5 mM) and SAHA+CETZOLE equimolar combination (2.5 mM) than the corresponding treatments with only SAHA (2.5 mM) or only CETZOLE-1 (2.5 mM).

FIGS. 11A-11C: The hybrid molecule shows selectivity rates for cancer cells over normal cells ranging between sensitivity to SAHA and CETZOLE-1. HY-1 has superior pharmacological effects on cancer cells (enhanced sensitivity of H522) vs the normal cells. NCI-H522, HCT-116, WI-38, and RPE cells grown in FBS media were treated with the inhibitors in a dose response manner FIG. 11A shows dose response graphs for NCI-H522, HCT-116, WI-38, and RPE cells treated with HY-1, SAHA, and CETZOLE-1. FIG. 11B shows the cell survival bar referring to a representative concentration of approximately 2 μM. Data are mean±SD (n=3) and expressed as fold decrease relative to the value for the control group (DMSO). Statistical analysis utilizing one-way ANOVA test *P<0.05, **P<0.01, ***P<0.001. FIG. 11C shows a table with IC₅₀±SD (n=3) values (μM) for HY-1, SAHA, and CETZOLE-1. Fold change¹ refers to the ratio of mean IC₅₀ (WI38) over mean IC₅₀ (NCI-H522). Fold change² refers to the ratio of mean IC₅₀ (RPE) over mean IC₅₀ (HCT-116).

FIGS. 12A-12D: The hybrid molecule shows attenuated effects on neuronal cells, accompanied by significant sensitivity of the NCI-H522 and HCT-116 cell lines. FIG. 12A shows viability of the SH-SY5Y cell line after 3 days of treatment with the amide series analogs at concentrations of 0.5×IC₅₀, 1×IC₅₀, 2×IC₅₀ of the corresponding ferroptosis or HDAC inhibitor controls on H522 cancer cell line. For HY-1, the tested concentrations are approximately 1×IC₅₀, 2×IC₅₀, and 4×IC₅₀ on H522 cancer cell line. FIG. 12B shows cell viability percentages of NCI-H522 and HCT-116 cells after 3 days of treatment with the amide series analogs at a concentration of 2.5 μM. The cell survival is shown as a % of average of DMSO treatment after triplicate experiment. FIG. 12C shows non-differentiated PC-12 cells have stem like properties, but when differentiated by nerve growth factor, they demonstrate neuronal behavior. FIG. 12D shows both differentiated and undifferentiated PC-12 cells proved to be resistant to ferroptosis induction. HY-1 behaved like the HDAC control. Data are mean±SD (n=3) and expressed as fold decrease relative to the value for the control group (DMSO). Statistical analysis utilizing one-way ANOVA test *P<0.05, **P<0.01, ***P<0.001.

FIG. 13: Flow cytometry data utilizing the C11-BODIPY^{581/591} probe for lipid peroxide quantification. NCI-H522 cells were treated with DMSO, SAHA (10 μM), HY-1 (10 μM), or HY-1 (10 μM)+Liproxstatin-1 (0.25 μM) for 6 h. Green is a measure of the levels of the oxidized form of the dye, proportional to the lipid peroxide levels.

FIGS. 14A-14B: Addition of ferroptosis inhibitor Liproxstatin-1 (0.25 μM) rescues the cells after one day of treatment with the hybrid molecules, but not after 4 days of treatment. FIG. 14A shows cell survival after one day of treatment with CETZOLE-1 (10 μM), SAHA (5 μM), HY-1 (5 μM), HY-2 (5 μM), HY-2-M (10 μM), HY-4 (20 μM), and HY-5 (20 μM) in the presence (0.25 μM) or absence of Liproxstatin-1. FIG. 14B shows cell survival % after four days of treatment with CETZOLE-1 (10 μM), SAHA (5 μM), HY-1 (5 μM), HY-2 (5 μM), HY-2-M (10 μM), HY-4 (2.5 μM), and HY-5 (20 μM) in the presence (0.25 μM) or absence of Liproxstatin-1. Data are mean±SD (n=3) and expressed as fold decrease relative to the value for the control group (DMSO). Statistical analysis utilizing one-way ANOVA test *P<0.05, **P<0.01, ***P<0.001.

FIGS. 15A-15B: Treatment of NCI-H522 cells with the hybrid molecules leads to hyper acetylation of histones and tubulin. FIG. 15A shows a Western blot analysis using acetyl-H3 (K9) and acetyl tubulin antibody. NCI-H522 cells were treated with the hybrid molecules or controls at the corresponding concentrations in the presence of Liproxstatin-1 (0.25 μM) for 3 days. For HY-1, similar results were obtained with a “pan-acetyl-lysine” antibody (shown in FIG. 15B).

FIGS. 16A-16C: Intercellular propagation of ferroptosis is more intense with the hybrid molecules. The addition of ferroptosis inhibitor Liproxastatin-1 makes the hybrid molecules have similar antimitotic affects with SAHA. FIG. 16A shows a time-lapse of microscopy of NCI-H522 cells treated with DMSO, SAHA (5 μM), CETZOLE-1 (20 μM), HY-1 (5 μM), and HY-1 (5 μM)+Liproxstatin-1 (0.25 μM). The images were captured every 12 min for a total of 300 pictures. FIG. 16B shows a Kaplan-Meier curve for the corresponding treatments of FIG. 16A. For these graphs cell death at certain time intervals (1 hr or 0.2 h) was considered as an event. On the left is a comparison of all treatments at time intervals of 1 h. On the right is a comparison of HY-1 and CETZOLE-1 at time intervals of 0.2 h. FIG. 16C shows flow cytometry data of fixed NCI-H522 cells stained with PI after treatment with DMSO, SAHA (5 μM), or HY-1 (5 μM) in the presence of Liproxstatin-1 (0.25 μM). The graph in FIG. 16C shows the % values of cells that are dead in G1, S, G2/M, or post-G2 phase for the corresponding time after treatment.

FIGS. 17A-17I: Docking scores for the designed library of analogs as well as for the controls SAHA and CETZOLE-1 on several HDAC isoforms. FIG. 17A shows an x-axis representing the entry ID based on the docking score, e.g., the compound with lowest docking score is entry 1. The y-axis represents the docking score which degreases from top to bottom. As a result of this representation, a diagonal can be drawn from top right to bottom left, a direction in which the binding ability increases. Thus, the best compounds for each HDAC isoform will be accumulated more towards the bottom left of each graph. FIGS. 17B-17I show the docking scores for seven different HDAC enzyme crystal structures downloaded from the Protein Data Bank (PDB) used: HDAC-1 PDB id: SICN (FIG. 17B), HDAC-2 PDB id: 4LXZ (FIG. 17C), HDAC-8 PDB id: 1W22 (FIG. 17D), HDAC-4 PDB id: 2VQM (FIG. 17E), HDAC-7 PDB id: 3C10 (FIG. 17F), HDAC-6-CD1 PDB id: 6UO2 (FIG. 17G), HDAC-6-CD2 PDB id: 5G0H (FIG. 17H), HDAC-10 PDB id: 6WBQ (FIG. 171). SAHA and HY-1 (colored red) showed similar behavior and constantly ranked among the best compounds (bottom left of the graphs). Each data point is colored based on the value of the docking score. Red represents the highest docking score while blue represents the lowest docking score in each data set. For HDACs 1, 2, 6 (CD2), and 10, no good binding poses of CETZOLE-1 were found. Thus, no docking scores are reported. All HDAC proteins used in this in-silico study are of human origin, except HDAC-6 CD1 and CD2, which originate from zebrafish.

FIG. 18: 3D representation of the binding motifs for two representative hybrid analogs on 7 different HDAC proteins. Compounds HY-1 (orange) and HY-2 (turquoise) bind to the active site for HDACi, with the hydroxamate binding to the zinc ion, the linker placed in the narrow tunnel, and the cap group placed at the rim area. HDAC-1 PDB id: SICN, HDAC-2 PDB id: 4LXZ, HDAC-8 PDB id: 1W22, HDAC-4 PDB id: 2VQM, HDAC-7 PDB id: 3C10, HDAC-6-CD1 PDB id: 6UO2, HDAC-6-CD2 PDB id: 5G0H, HDAC-10 PDB id: 6WBQ.

FIGS. 19A-19B: Summary of data from the one dose and five dose assays performed by NCI-60 DTP. FIG. 19A shows the mean Inhibition (%) values for hybrids and HDAC controls. FIG. 19B shows average GI₅₀ values for the compounds tested on the five-dose assay. Both one dose and five dose assays were performed as single experiments and not in multiple replicates.

FIG. 20: NCI-60 one dose data for HDAC controls, HY-(1-5), and SAHA. Most of the tested cell lines showed enhanced sensitivity to the hybrid molecules over the HDAC controls. HY-1 demonstrated the best profile. A plethora of cell lines showed remarkable sensitivity to HY-1 over SAHA. Data for SAHA was obtained from the public data base of NCI. SAHA (NSC 759852)-NSC Cancer Chemotherapy National Service Center number assigned by NCI. Data are Growth % values.

FIG. 21: Heat map of GI₅₀ values (μM) for HY-1, HY-2, HY-3, HY-5, SAHA, HC-1, and HC-5 in the NCI-60 five dose assay. Crossed boxes denote data not determined. White boxes indicate GI₅₀>4 μM and are not included for better resolution.

FIG. 22: Scheme 6, depicting the synthesis of hybrid molecules by combining the CETZOLE-1 analog (1) and the SAHA analog (3).

FIG. 23: Non-limiting example hybrid molecules (ferroptotic HDACi), along with inactive controls, ferroptotic inducers, and HDAC inhibitors.

FIG. 24: Scheme 7, depicting the synthesis of analogs containing a cinnamate linker (“cinnamate analogs”).

FIG. 25: Table showing IC₅₀±SD (n=3) values for cinnamate analogs on NCI-H522 and HCT-116 cell lines.

FIG. 26: Cell survival of NCI-H522 cells treated with DMSO, SAHA (5 mM), 10 (5 mM), or CETZOLE-1 (10 mM) in combination with none, one, or two of the cell death inhibitors Liproxtastin-1 (0.25 mM), Z-VAD-FMK (10 mM), and Necrostatin-5 (10 mM).

FIG. 27: Results of a chain elongation study of the linker, connecting the ferroptotic cap group with the ZBG. The results show that HY-1 demonstrated a superior cytotoxicity profile compared to its shorter or longer analogs.

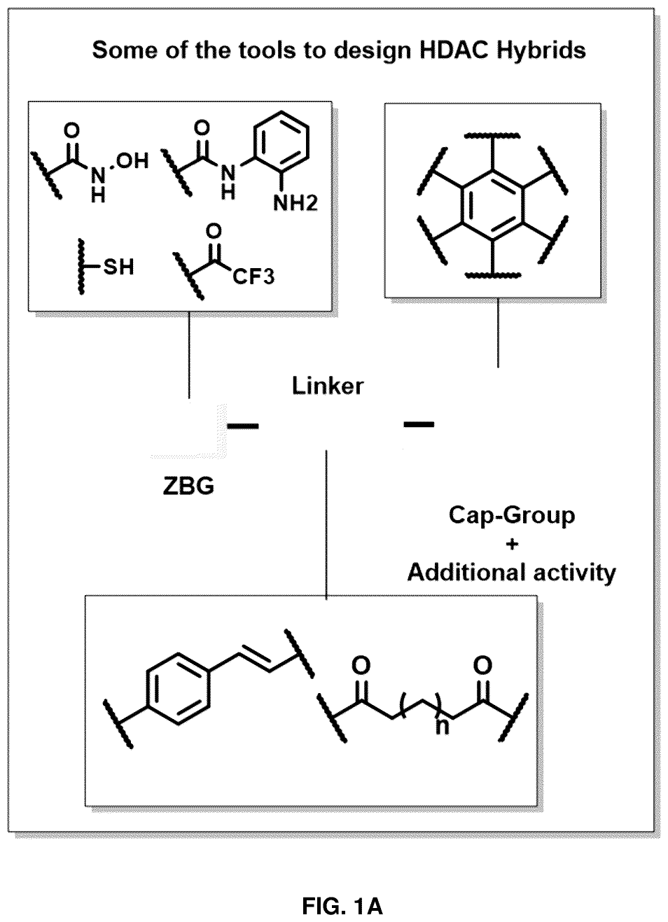

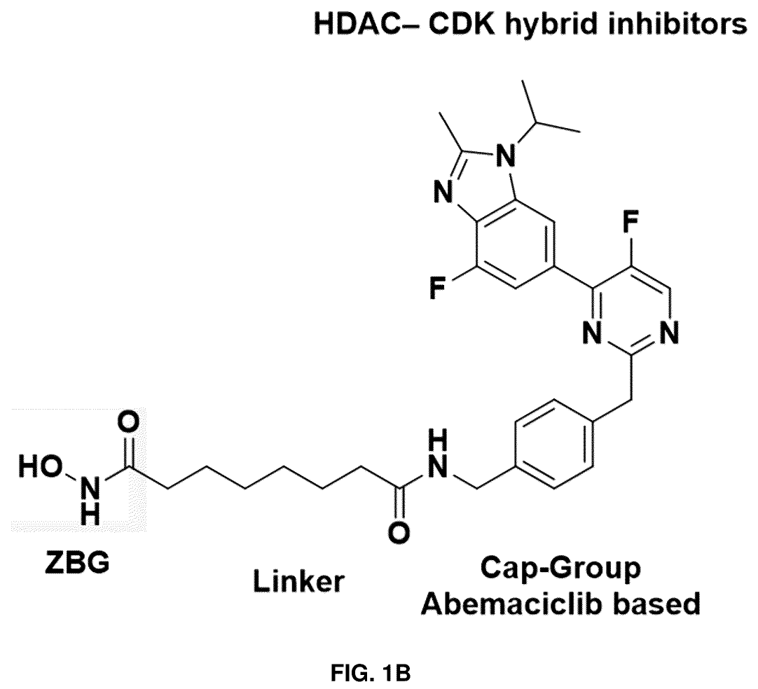

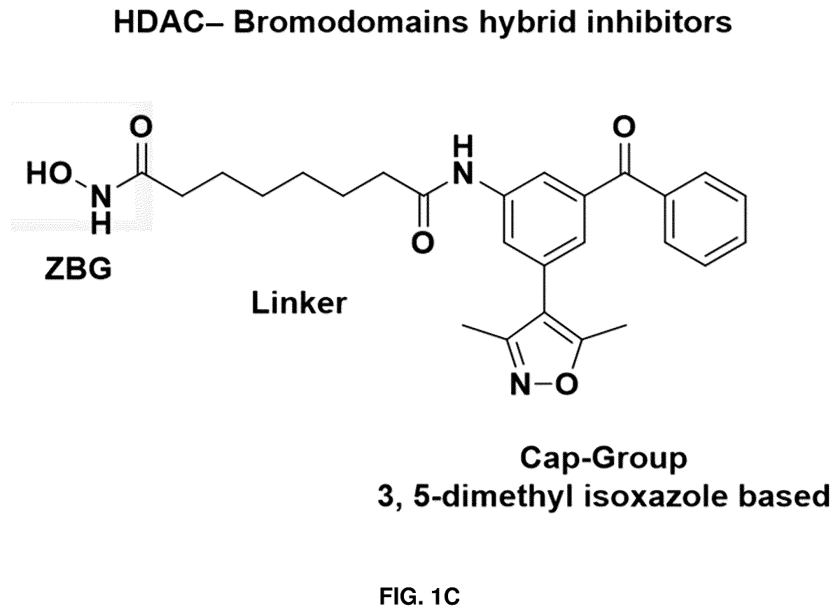

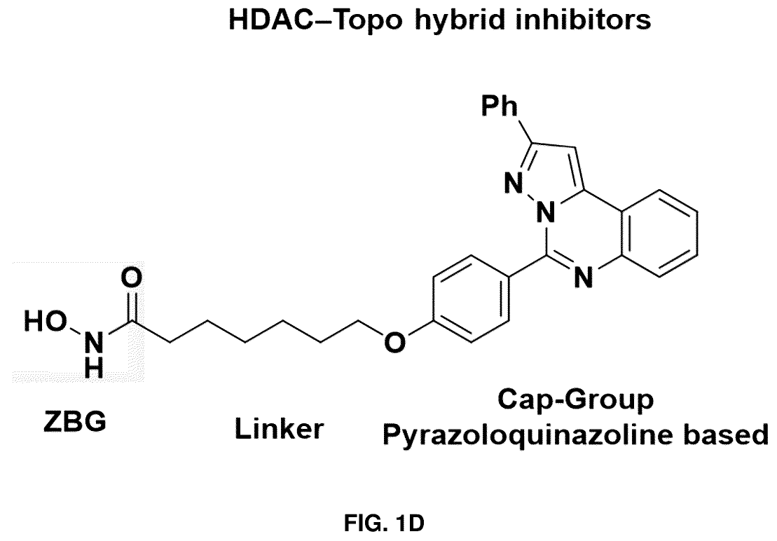

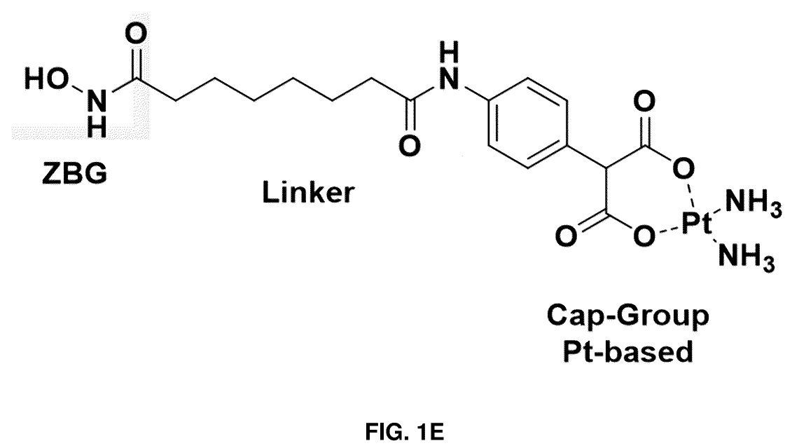

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

The lack of diversity in activating different cell death mechanisms is a major disadvantage of conventional hybrids molecules, because many cancer cells either have defective apoptosis pathways, or can dysregulate different steps in these pathways leading to drug resistance. Accordingly, the design and synthesis of hybrid molecules that combine apoptosis with other cell death machinery is an attractive alternative. In particular, combinations using drugs to sensitize cancer progenitor cells which are insensitive to standard therapies may be especially useful in improving the clinical outcome of chemotherapy.

One non-apoptotic cell death mechanism is known as ferroptosis. The hallmark of ferroptosis is iron-dependent lipid peroxidation, which, in combination with defective lipid peroxide repair mechanisms, leads to programmed cell death without requiring caspase activity. Ferroptosis is a nonapoptotic cell death mechanism characterized by a rapid increase in ROS leading to membrane lipid peroxidation. There are currently no ferroptosis inducers in clinical use.

Synthetic agents can induce ferroptosis by inhibiting cellular components that are crucial for maintaining an intracellular reductive environment such as system Xc⁻ (erastin, sorefinib) or GPX4 (RSL3, ML210). In addition, several other processes such as ferritinophagy, epithelial-to-mesenchymal transition (EMT), and glutamine and iron metabolism modulate ferroptotic cell death. One important and unique feature of ferroptosis that makes it attractive for drug development is the enhanced sensitivity of mesenchymal cells to ferroptotic agents due to their high dependency on pathways that lead to lipid peroxidation quenching. The mesenchymal state has been associated with drug resistance and cell migration and, thus, enhanced and selective killing of this subpopulation of cells will lead to reduced drug resistance and tumor metastasis, especially when combined with another cell death mechanism such as apoptosis.

The combined effects of HDACi and ferroptosis inducers are highly useful with respect to cancer treatment and reduced neurotoxicity. For example, HDACi increase the sensitivity of cells to ferroptosis, leading to synergetic killing of cancer cells. In addition, the neuroprotective effects of HDACi can be an added benefit of such drug combinations by attenuating the neurotoxic effects of ferroptotic agents. Expression of distinct HDAC isoforms in neurons versus cancer cells is believed to explain this difference. Although the exact mechanisms by which HDAC inhibitors enhance ferroptosis in cancer cells is yet not fully understood, it is beleived that HDAC inhibition induces EMT and alters cellular iron homeostasis.

Combination therapies using a mixture of two or more anticancer agents are used to overcome the limitations of monotherapy, including drug resistance. However, differences in pharmacokinetic properties and spatio-temporal biodistbution of the drugs used limit the scope of combination therapies. Provided herein are hybrid molecules which act as dual-mechanism anticancer agents to overcome the limitations of conventional combination therapies. The hybrid molecules incorporate pharmacophores from different drugs, some acting by apoptotic and nonapoptotic mechanisms. Advantageously, the hybrid molecules may maintain the pharmacodynamic profiles of individual drugs while ensuring their uniform spatiotemporal distribution.

Provided herein are hybrid molecules that are highly potent anticancer agents and which incorporate the pharmacophores of a ferroptosis inducer and a histone deacetylase (HDAC) inhibitor. These hybrid molecules may be referred to as ferroptosis-HDAC inhibitor hybrids, or ferroptotic HDACis.

A ferroptotic agent has the ability to kill cancer stem cells responsible for tumor metastasis and therefore prevent tumor metastasis. An HDAC inhibitor is not effective on solid tumors. However, the hybrid molecules have the ability to extend application to solid tumors. Combination of ferroptosis with the apoptotic cell death mechanism of HDAC inhibitors have the ability reduce drug resistance and tumor metastasis. The hybrid molecules retain the pharmacodynamic profiles of the individual drugs while ensuring their uniform spatiotemporal distribution.

In accordance with the present disclosure, provided are hybrid molecules that are capable of inducing ferroptosis in cancer cells while maintaining HDAC inhibitory activity. In general, the hybrid molecules include a zinc-binding group (ZBG) attached to a linker, and a cap group attached to the linker. The ZBG group includes a hydroxamic acid or hydroxamate. However, other ZBGs are possible and encompassed within the scope of the present disclosure. The linker may be, or may include, a short aliphatic chain, such as from about 3 carbons to about 6 carbons. However, other linkers are possible, as described in more detail below, and encompassed within the scope of the present disclosure. The cap group of the hybrid molecules may include a terminal alkyne bonded to a thiazole, or more specifically a terminal alkyne at the 2-position of a thiazole ring, which is also referred to as a CETZOLE. The cap group may further include additional functional groups or moieties, such as an amide.

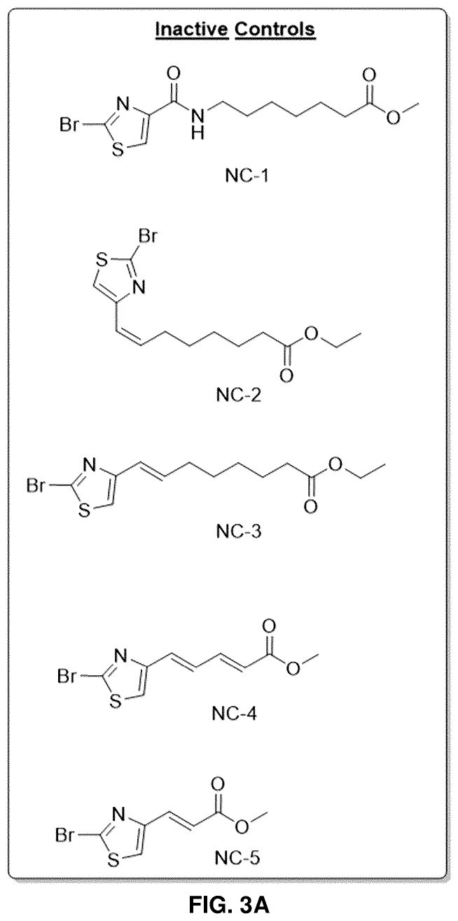

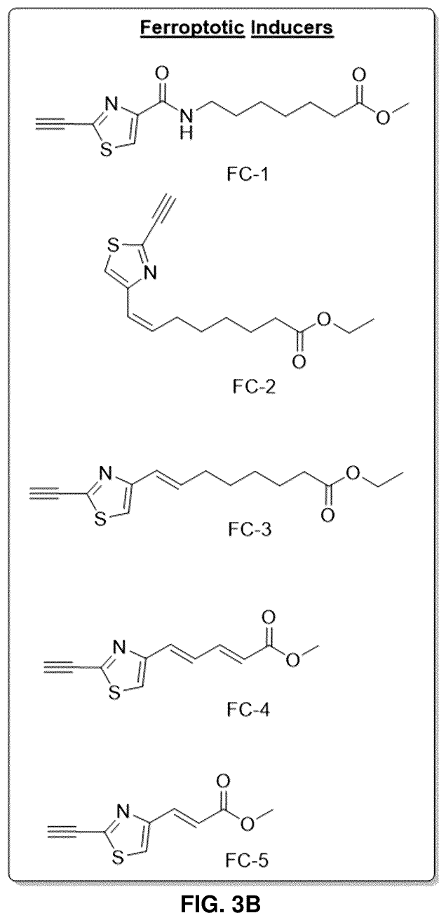

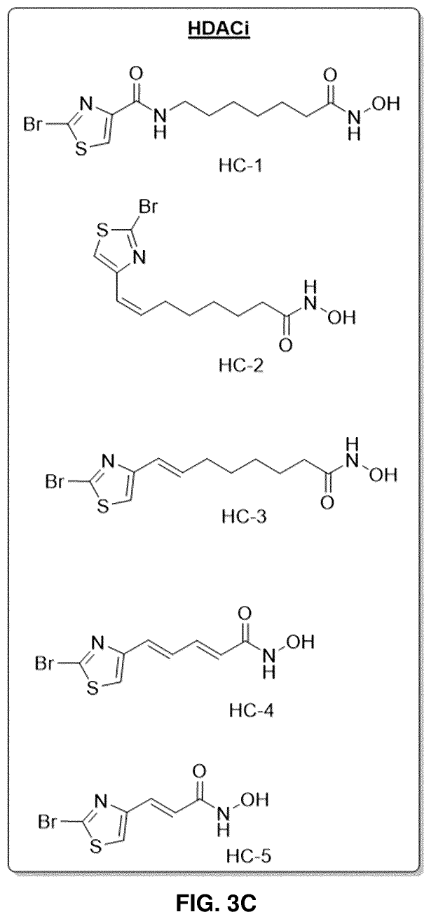

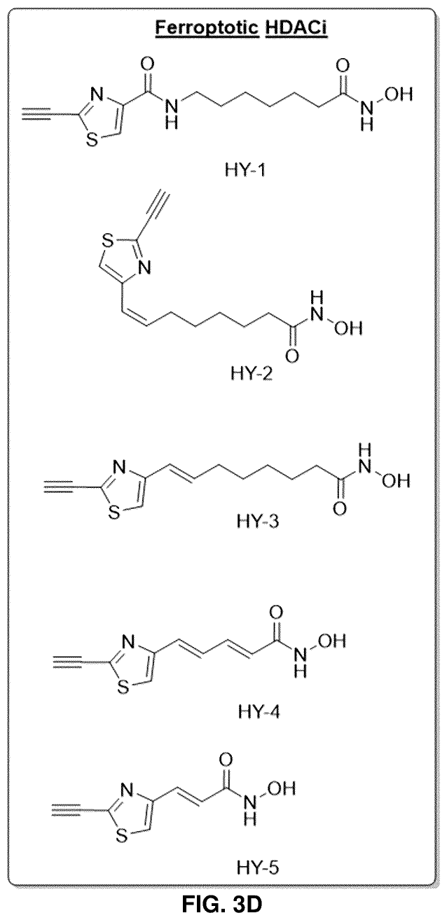

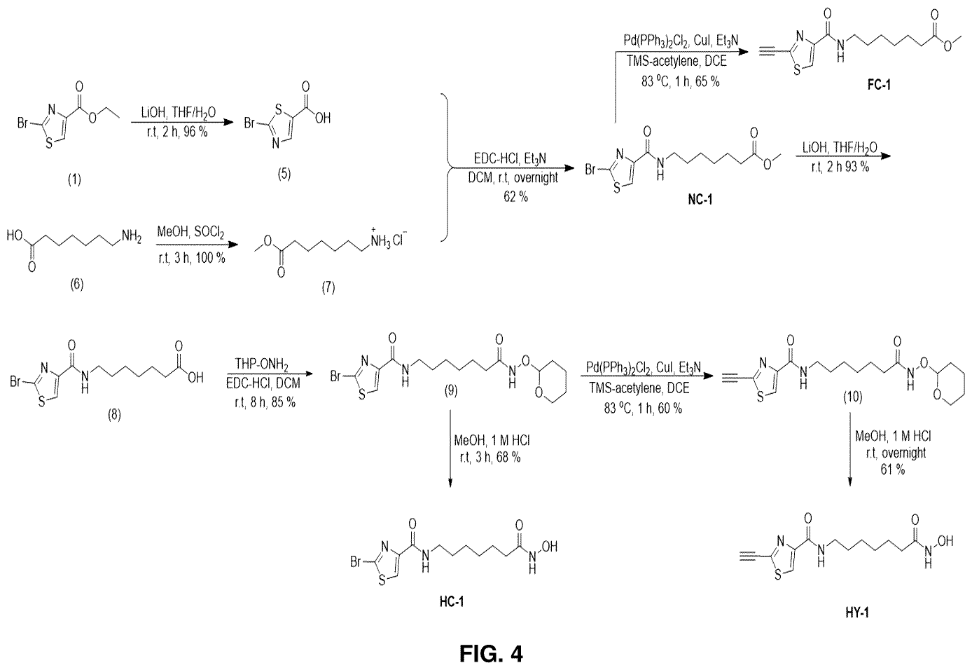

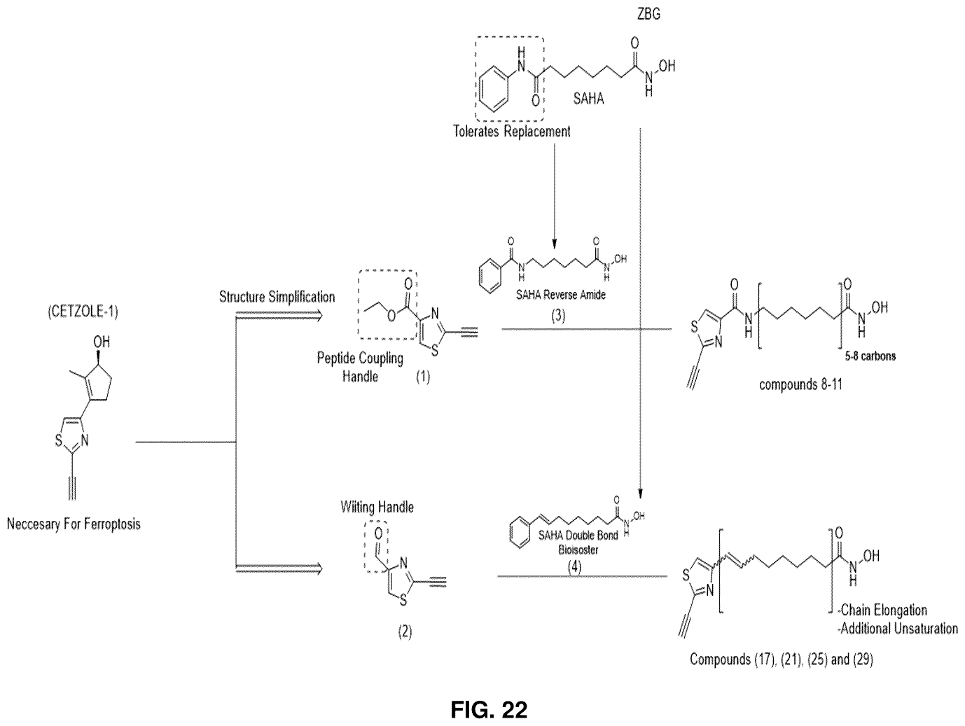

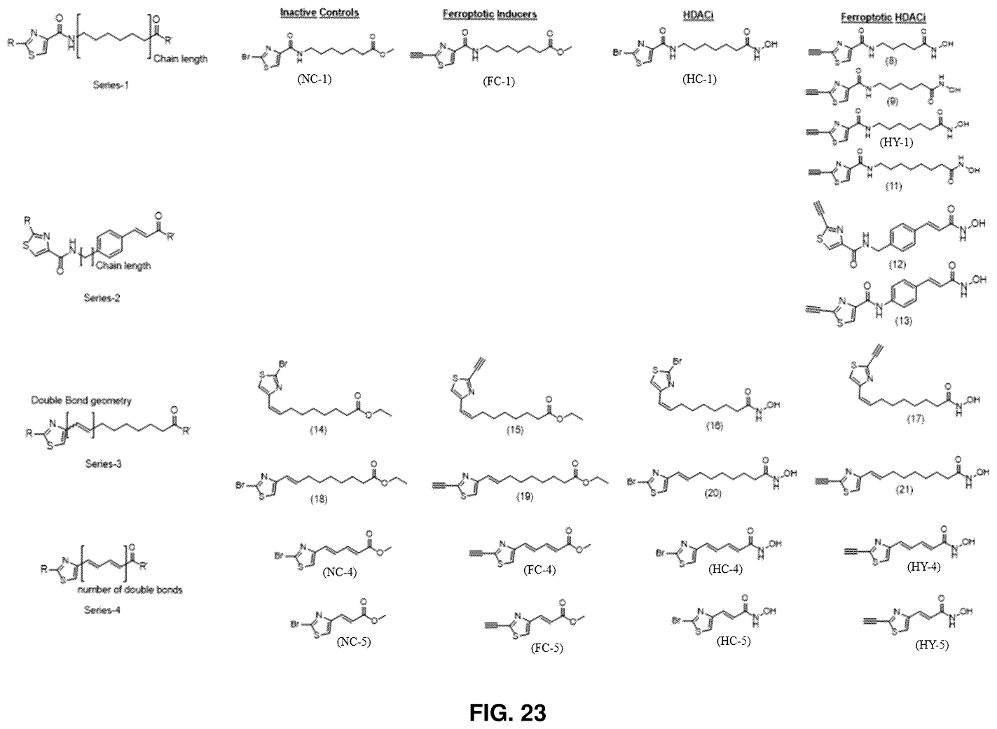

In some embodiments, the hybrid molecules have the general structural formula of Formula A:

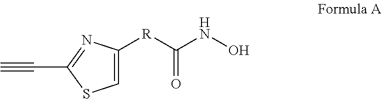

where R is an alkyl, alkenyl, amidoalkyl, amidoalkenyl, arylalkyl, arylalkenyl, or amidoarylalkenyl group. For example, R may be a (C1-C6) alkyl or (C1-C6) alkenyl. As another example, R may be an amidohexyl. In some examples, R includes an aliphatic chain of from 4 to 6 carbons, and may further include either an alkenyl group or an amido group. Non-limiting example hybrid molecules are HY-1, HY-2, HY-3, HY-4, and HY-5, which are depicted in FIG. 3D. Other non-limiting example hybrid molecules are depicted in FIG. 23. These non-limiting example hybrid molecules can be synthesized according to Schemes 1-4, shown in FIGS. 4-7, as well as in FIG. 22, which shows the synthesis of hybrid molecules by combining the CETZOLE-1 analog with a SAHA analog.

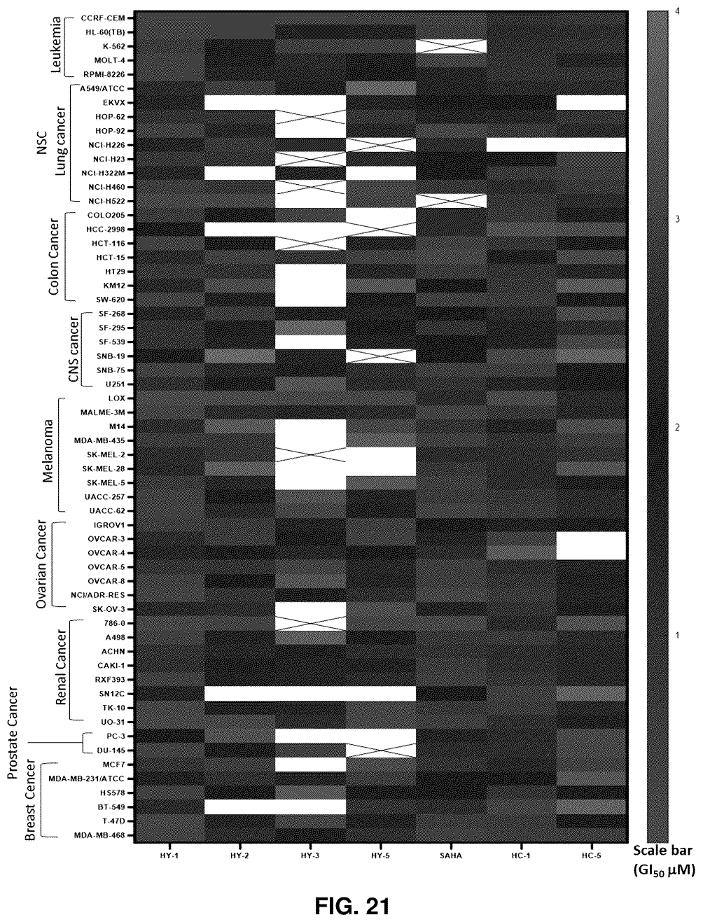

As demonstrated in the examples herein, the hybrid molecules are useful as anticancer agents. Certain hybrid molecules have significant activity and selectivity against a wide range of cancers. For example, HY-1 is cytotoxic to renal cancer, colon cancer, leukemia, ovarian cancer, breast cancer, lung cancer, melanoma, and CNS cancer. (FIG. 21.) HY-2 is cytotoxic to renal cancer, leukemia, ovarian cancer, breast cancer, lung cancer, melanoma, and CNS cancer. (FIG. 21.) HY-3 is cytotoxic to renal cancer, leukemia, ovarian cancer, lung cancer, melanoma, or CNS cancer. (FIG. 21.) HY-5 is cytotoxic to renal cancer, leukemia, ovarian cancer, breast cancer, lung cancer, melanoma, and CNS cancer. (FIG. 21.)

In some embodiments, the hybrid molecules are more active than SAHA (Vorinostat). The hybrid molecules kill cancer cells by ferroptosis (nonapoptotic) and HDAC inhibition (apoptoic) mechanisms, and therefore can prevent drug resistance. In some embodiments, the hybrid molecules are effective on cancer stem cells, and therefore can prevent cancer metastasis.

As noted above, hybrid molecules combine the benefits of combinatorial treatment with homogeneous spatiotemporal distribution. HDAC inhibitors are an attractive class of cytotoxic agents for the design of hybrid molecules. Several HDAC hybrids have emerged over the years but none combines HDAC inhibition with ferroptosis, a combination which leads to enhanced cytotoxicity and attenuated neuronal toxicity. In accordance with the present disclosure, the pharmacophores of an HDAC inhibitor and a ferroptosis inducer have been combined to design dual-mechanism hybrid molecules which induce ferroptosis and inhibit HDAC proteins. As described in the examples herein, the involvement of both mechanisms in cytotoxicity was confirmed by a series of biological assays where hallmarks of both mechanisms were investigated. The cytotoxic effects were evaluated in a series of cancer and neuronal cell lines. In these examples, analog HY-1 demonstrated the best cytotoxic profile with GI₅₀ values as low as 20 nM.

Further provided are ferroptotic inducers which include the CETZOLE cap group like the hybrid molecules, and similar linkers, but do not include the same ZBG group. The ferroptotic inducers, which may also be referred to as ferroptosis inducers, have the general structural formula of Formula B:

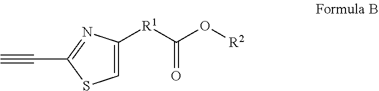

where R¹ is an alkyl, alkenyl, amidoalkyl, amidoalkenyl, arylalkyl, arylalkenyl, or amidoarylalkenyl group; and R² is alkyl. Thus, the ferroptosis inducers include an ester in the ZBG group. Non-limiting examples of ferroptotic inducers include FC-1, FC-2, FC-3, FC-4, and FC-5, which are depicted in FIG. 3B. These non-limiting example ferroptotic inducers can be synthesized according to Schemes 1-4, shown in FIGS. 4-7.

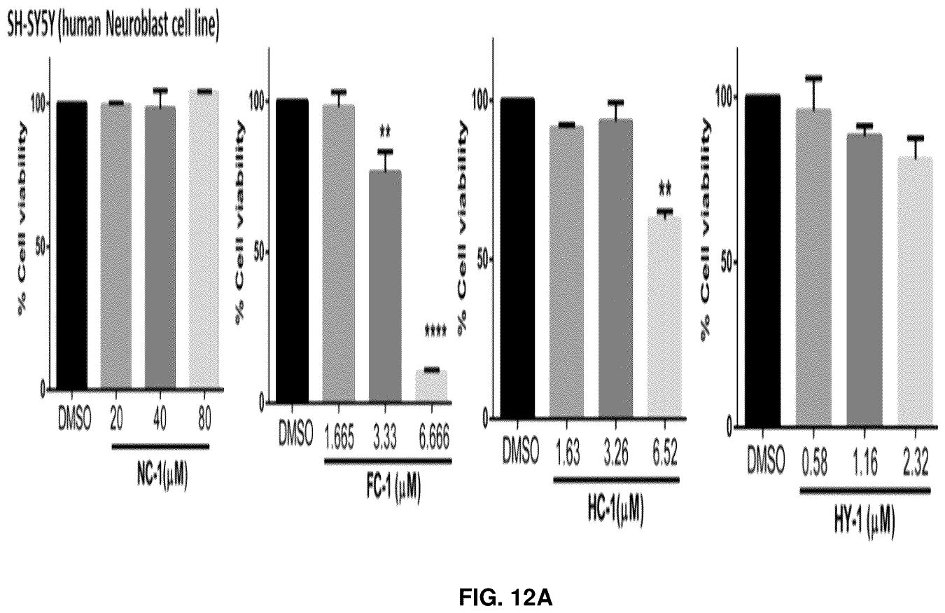

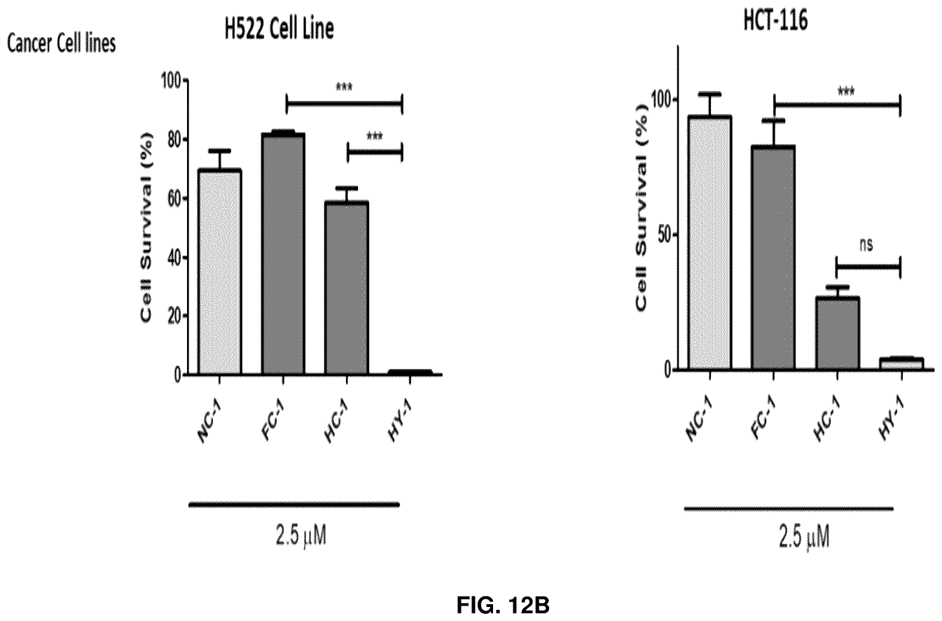

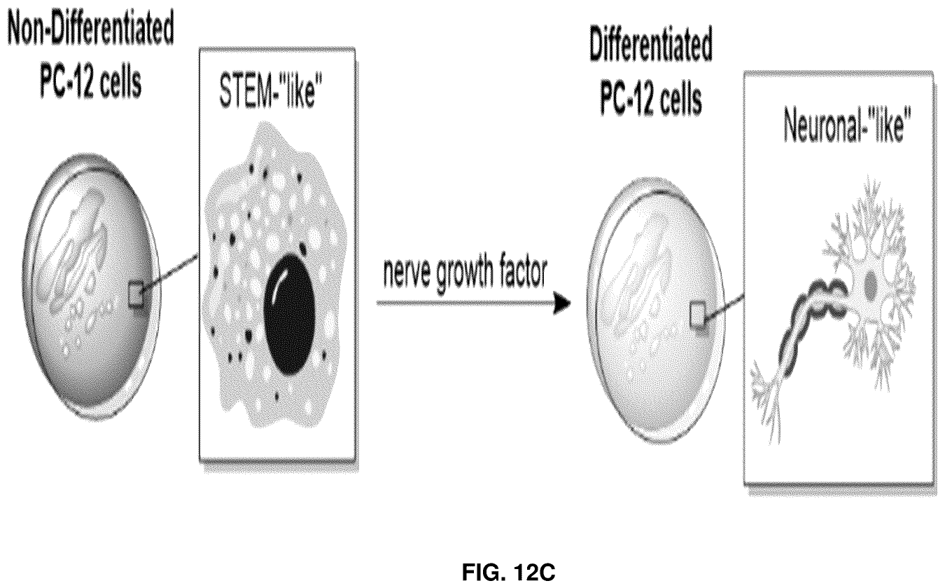

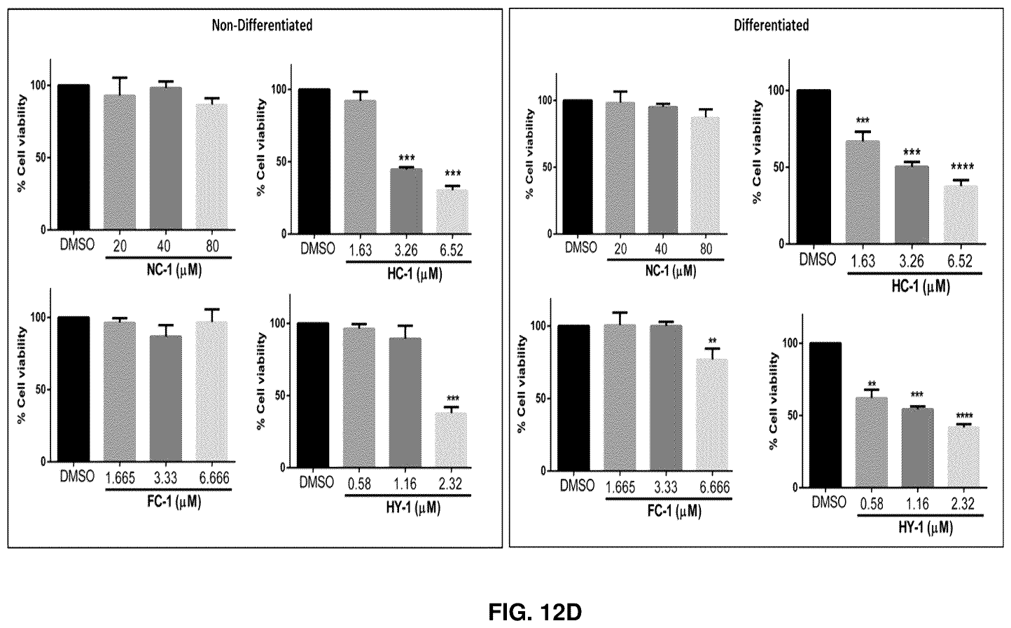

As demonstrated in the examples herein, the ferroptotic inducers are useful against certain cancers. For example, as shown in FIG. 12A, the ferroptotic inducer FC-1 has significant neurotoxicity against human neuroblastoma cells. The ferroptotic inducers are useful against cancers that are susceptible to cell death through the ferroptosis mechanism.

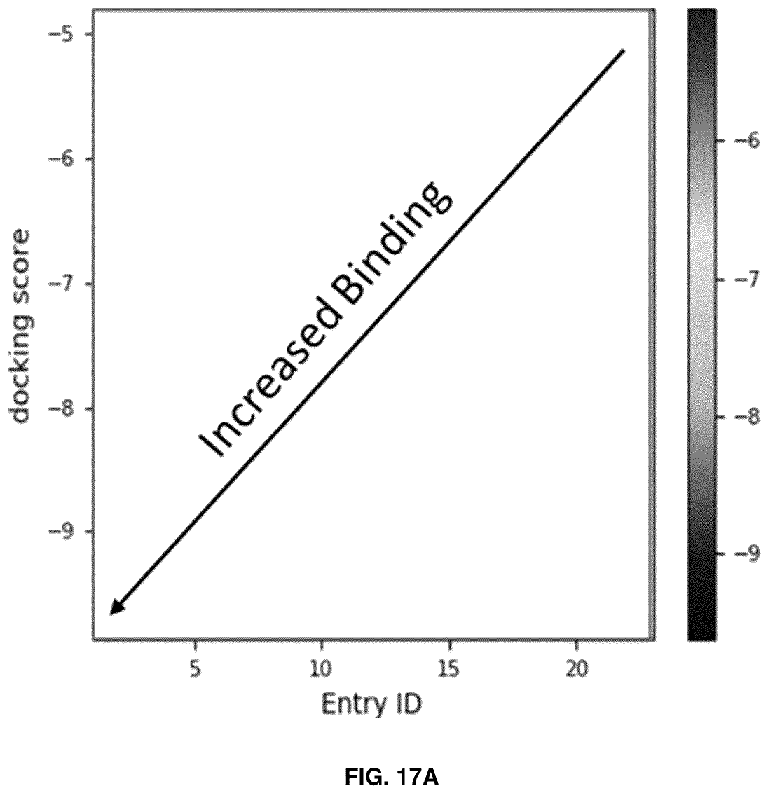

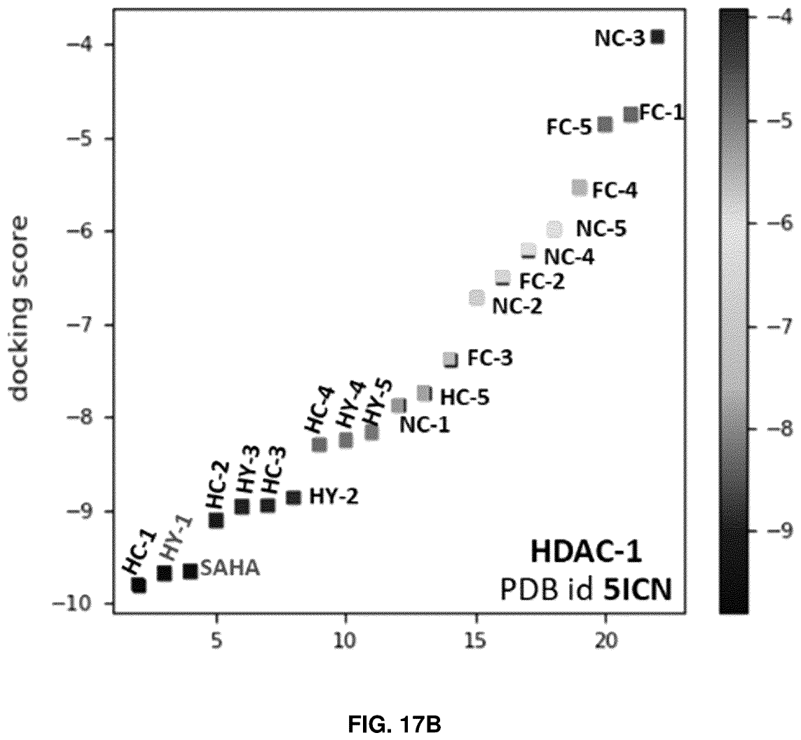

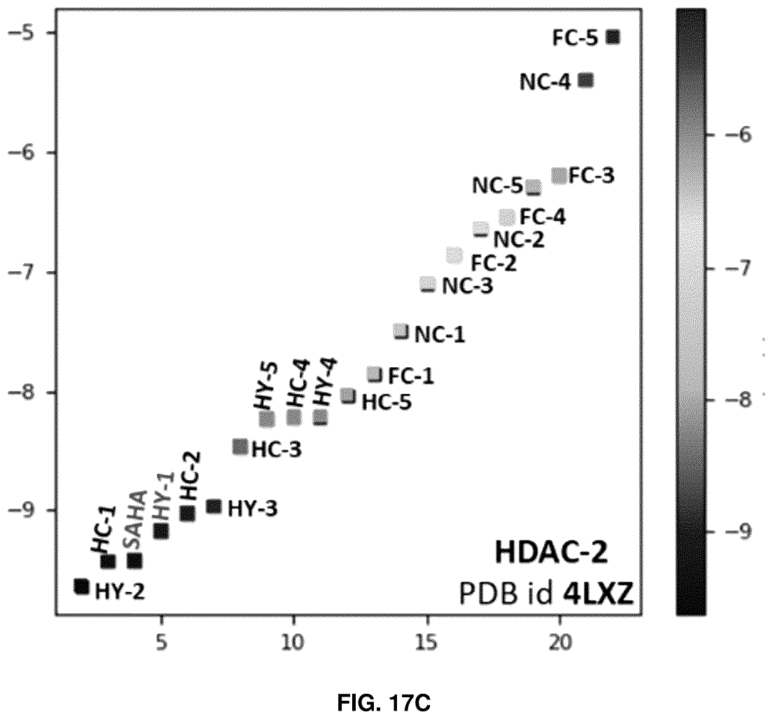

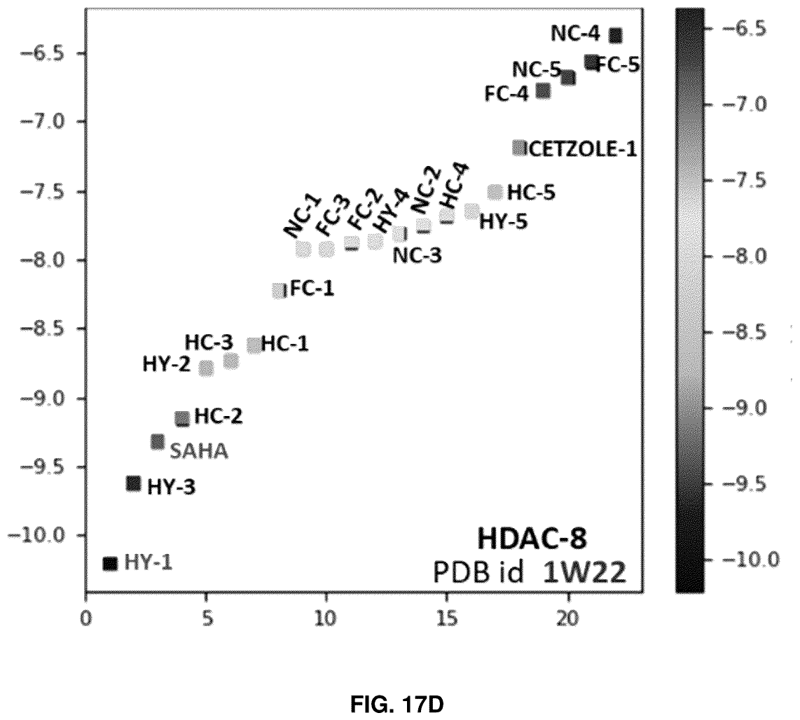

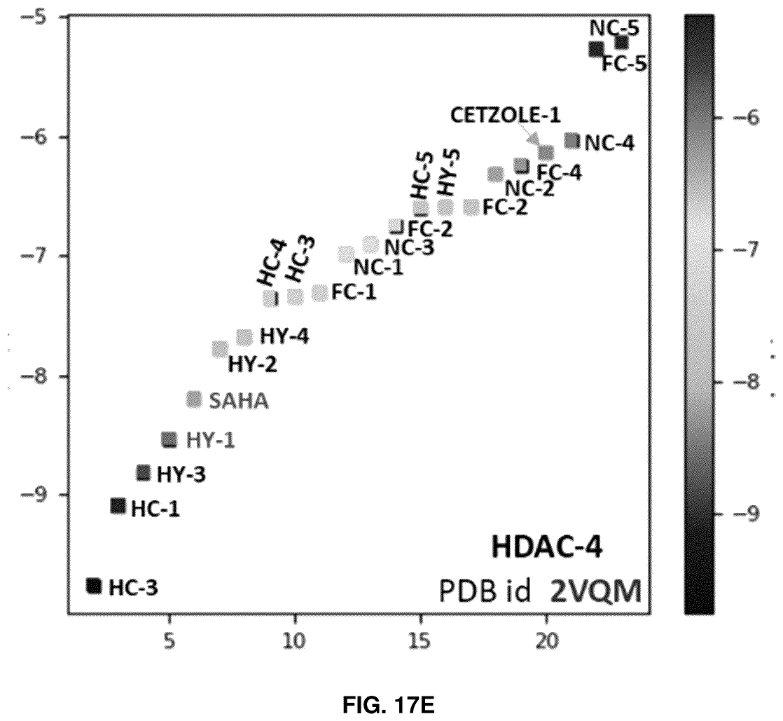

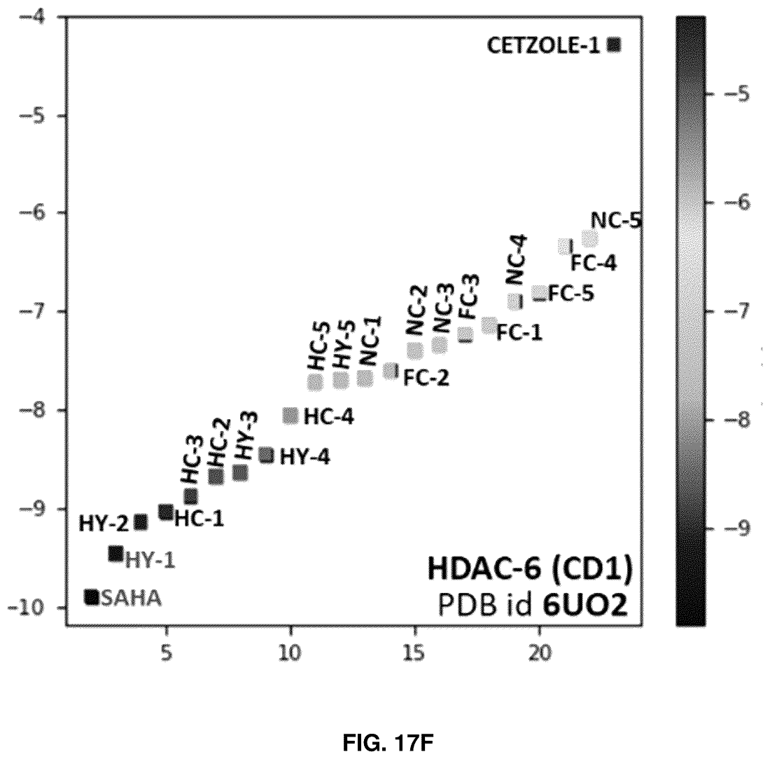

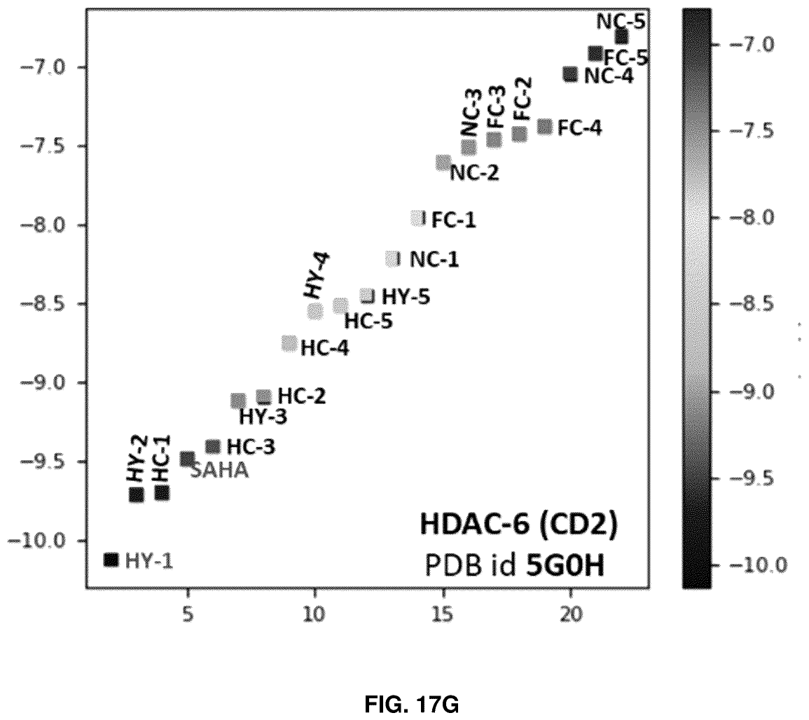

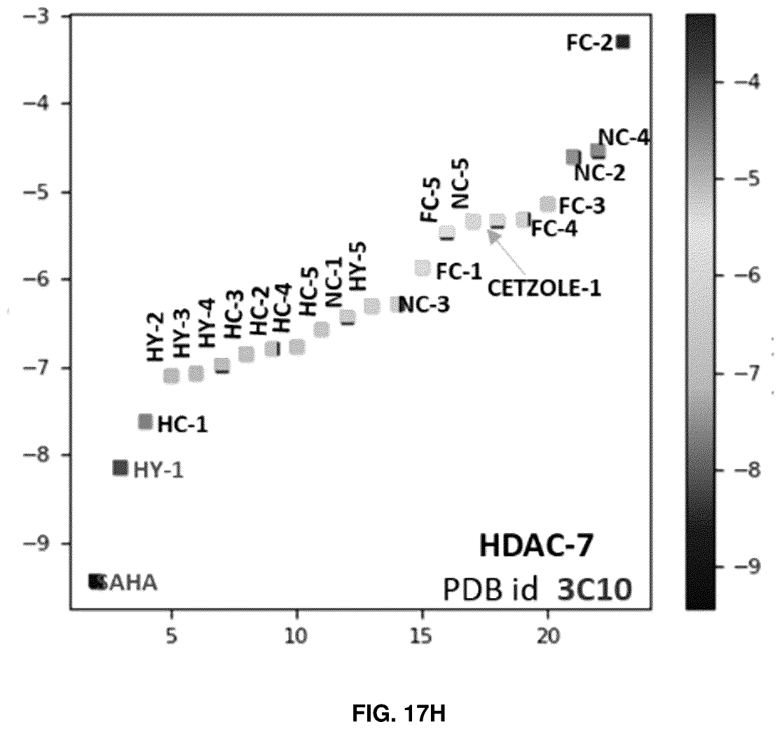

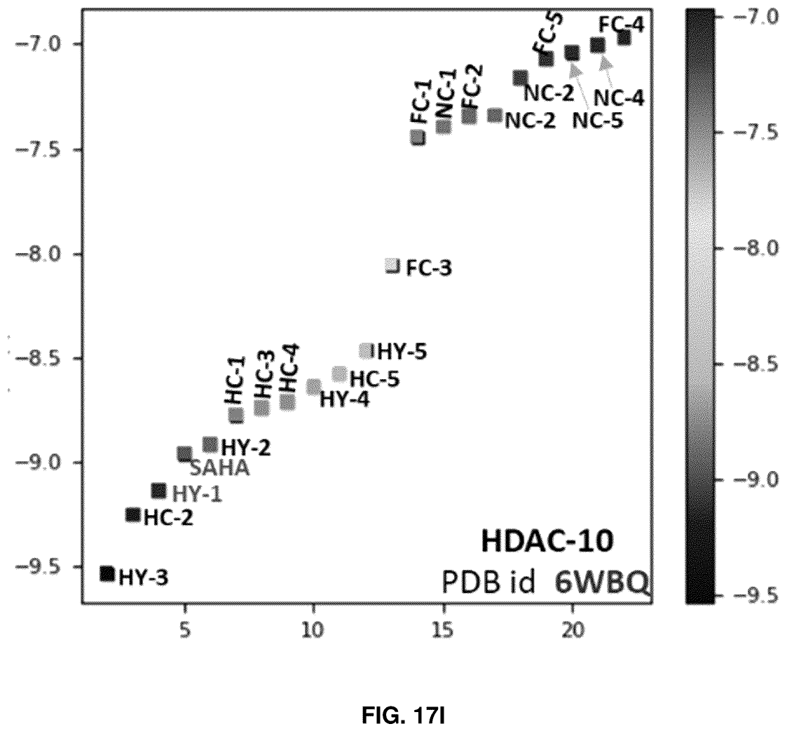

Further provided are HDAC inhibitors that include a hydroxamate ZBG like the hybrid molecules, and a similar linker connected to a thiazole, but do not include the terminal alkyne that the hybrid molecules include. The HDAC inhibitors have the general structural formula of Formula C:

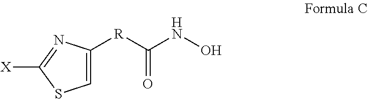

where X is a halide, and R is an alkyl, alkenyl, alkynyl, amidoalkyl, amidoalkenyl, arylalkyl, arylalkenyl, or amidoarylalkenyl group. In some embodiments, X is Br. R may be, for example, a (C1-C6) alkyl or (C1-C6) alkenyl. As another example, R may be an amidohexyl. In some examples, R includes an aliphatic chain of from 4 to 6 carbons, and may further include either an alkenyl group or an amido group. Non-limiting examples of HDAC inhibitors are HC-1, HC-2, HC-3, HC-4, and HC-5, depicted in FIG. 3C. These non-limiting example HDAC inhibitors can be synthesized according to Schemes 1-4, depicted in FIGS. 4-7. As demonstrated in the examples herein, the HDAC inhibitors are useful at inhibiting HDAC (FIG. 17), and are cytotoxic to certain cancers such as leukemia (FIG. 21).

Pharmaceutical compositions of the present disclosure may comprise an effective amount of a hybrid molecule, ferroptotic inducer, or HDAC inhibitor (an “active compound” or “active ingredient”), or combination thereof, optionally with additional agents, dissolved or dispersed in a pharmaceutically acceptable carrier, optionally with an additional cancer therapeutic drug. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515, and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form must be sterile and must be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.) and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the compounds and compositions described herein are useful for treating cancers or killing cancer cells. As described herein, the compounds and compositions herein can be used in combination therapies. That is, the compounds and compositions can be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active compound in a way that the therapeutic effects of the first administered procedure or drug is not entirely disappeared when the subsequent procedure or drug is administered.

In some embodiments, the hybrid molecule, ferroptotic inducer, or HDAC inhibitor is part of a combination therapy with a chemotherapeutic agent. Suitable chemotherapeutic agents include, but are not limited to: taxane compounds, such as paclitaxel; platinum coordination compounds; topoisomerase I inhibitors, such as camptothecin compounds; topoisomerase II inhibitors, such as anti-tumor podophyllotoxin derivatives; anti-tumor vinca alkaloids; anti-tumor nucleoside derivatives; alkylating agents; anti-tumor anthracycline derivatives; HER2 antibodies; estrogen receptor antagonists or selective estrogen receptor modulators; aromatase inhibitors; differentiating agents, such as retinoids, and retinoic acid metabolism blocking agents (RAMBA); DNA methyl transferase inhibitors; kinase inhibitors; farnesyltransferase inhibitors; HDAC inhibitors, or other inhibitors of the ubiquitin-proteasome pathway; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide, and trimethylomelamine; acetogenins; camptothecins, such as the synthetic analog topotecan; cryptophycins; nitrogen mustards, such as chlorambucil; nitrosoureas; bisphosphonates; mitomycins; epothilones; maytansinoids; trichothecenes; retinoids, such as retinoic acid; pharmaceutically acceptable salts, acids and derivatives of any of the above; and combinations thereof. Non-limiting examples of specific chemotherapeutic agents include erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethyl-ethanamine, NOLVADEX®, ISTUBAL®, VALODEX®), doxorubicin (ADRIAMYCIN®), Akti-1/2, HPPD, rapamycin, lapatinib (TYKERB®, Glaxo SmithKline), oxaliplatin (ELOXATIN®, Sanofi), bortezomib (VELCADE®, Millennium Pharm.), sutent (SUNITINIB CD, SU11248, Pfizer), letrozole (FEMARA®, Novartis), imatinib mesylate (GLEEVEC®, Novartis), XL-518 (MEK inhibitor, Exelixis, WO 2007/044515), ARRY-886 (MEK inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), ABT-869 (multi-targeted inhibitor of VEGF and PDGF family receptor tyrosine kinases, Abbott Laboratories and Genentech), ABT-263 (Bcl-2/Bcl-xL inhibitor, Abbott Laboratories and Genentech), PTK787/ZK 222584 (Novartis), fulvestrant (FASLODEX®, AstraZeneca), leucovorin (folinic acid), lonafamib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifamib (ZARNESTRA™, Johnson & Johnson), capecitabine (XELODA®, Roche), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thioTepa and cyclosphosphamide (CYTOXAN®, NEOSAR®), bullatacin, bullatacinone, bryostatin, callystatin, CC-1065 (including its adozelesin, carzelesin and bizelesin synthetic analogs), cryptophycin 1, cryptophycin 8, dolastatin, duocarmycin (including the synthetic analogs, KW-2189 and CB1-TM1), leutherobin, pancratistatin, sarcodictyin, spongistatin, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, clodronate, esperamicin, neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK® polysaccharide complex (JHS Natural Products, Eugene, Oreg.), razoxane, rhizoxin, sizofuran, spirogermanium, tenuazonic acid, triaziquone, 2,2′,2″-trichlorotriethylamine, T-2 toxin, verracurin A, roridin A, anguidine, urethane, vindesine, dacarbazine, mannomustine, mitobronitol, mitolactol, pipobroman, gacytosine, arabinoside (“Ara-C”), cyclophosphamide, thioTepa, 6-thioguanine, mercaptopurine, vinblastine, etoposide (VP-16), ifosfamide, mitoxantrone, vincristine, vinorelbine (NAVELBINE®), novantrone, teniposide, edatrexate, daunomycin, aminopterin, ibandronate, CPT-11, topoisomerase inhibitor RFS 2000, and difluoromethylomithine (DMFO), paclitaxel, 5-fluorouracil, abraxane (paclitaxel albumin-stabilized nanoparticle formulation), afinitor (everolimus), erlotinib hydrochloride, everolimus, gemcitabine hydrochloride, oxaliplatin (eloxatin), capecitabine (xeloda), cisplatin, irinotecan (camptosar), colinic acid (leucovorin), folfox (folinic acid, 5-fluorouracil, and oxaliplatin), folfirinox (folinic acid, 5-fluorouracil, irinotecan, and oxaliplatin), nab-paclitaxel with gemcitabine, metformin, digoxin, and simvastatin.

In some embodiments, the hybrid molecule, ferroptotic inducer, or HDAC inhibitor is part of a combination therapy with an immunotherapeutic agents. Non-limiting examples of immunotherapeutic agents include nivolumab, pembrolizumab, rituximab, durvalumab, cemiplimab, and combinations thereof.

In some embodiments, the hybrid molecule, ferroptotic inducer, or HDAC inhibitor is part of a combination therapy with a hormonal therapeutic agent. Non-limiting examples of hormonal therapeutic agents include anastrozole, exemestane, letrozole, tamoxifen, raloxifene, fulvestrant, toremifene, gosrelin, leuprolide, triptorelin, apalutamide, enzalutamide, darolutamide, bicalutamide, flutamide, nilutamide, abiraterone, ketoconazole, degarelix, medroxyprogesterone acetate, megestrol acetate, mitotane, and combinations thereof.

### EXAMPLES

These examples describe the design, general synthetic route, in silico, in vitro, and cellular evaluation of the hybrid molecules described herein.

### Rational Design of Ferroptosis-HDACi Hybrid Molecule

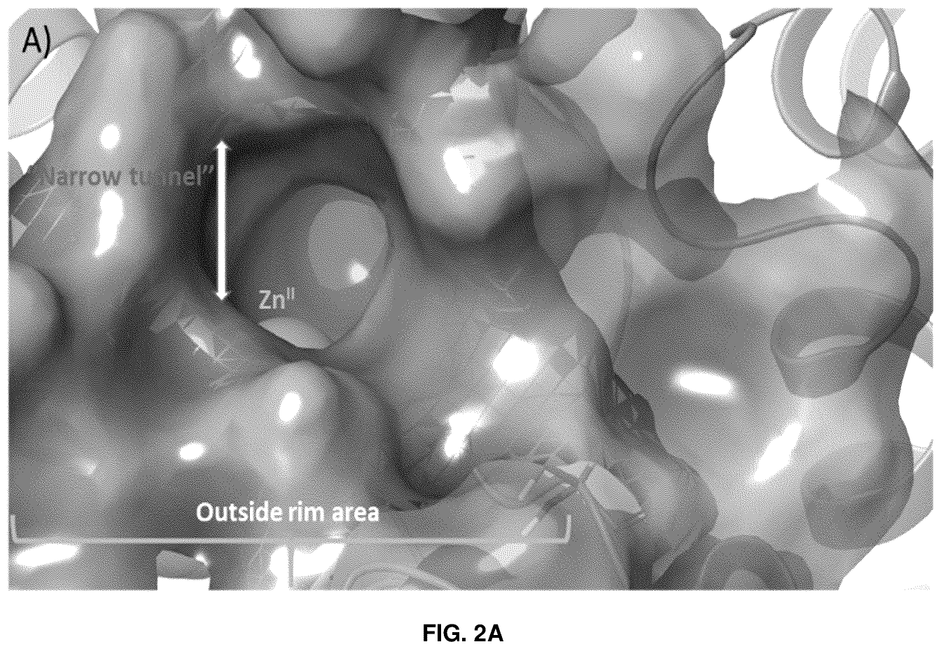

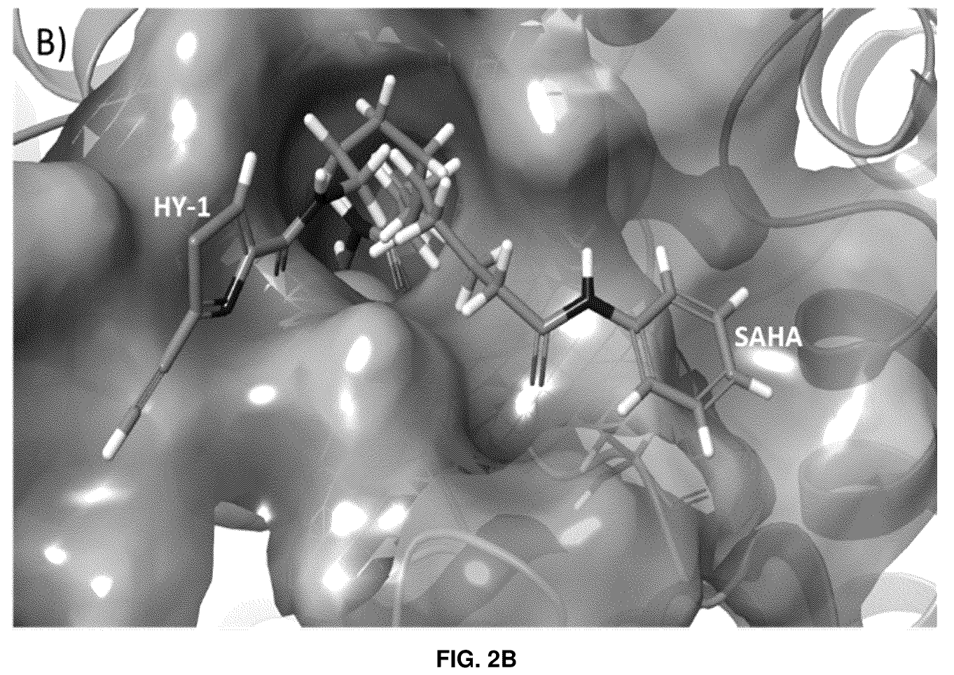

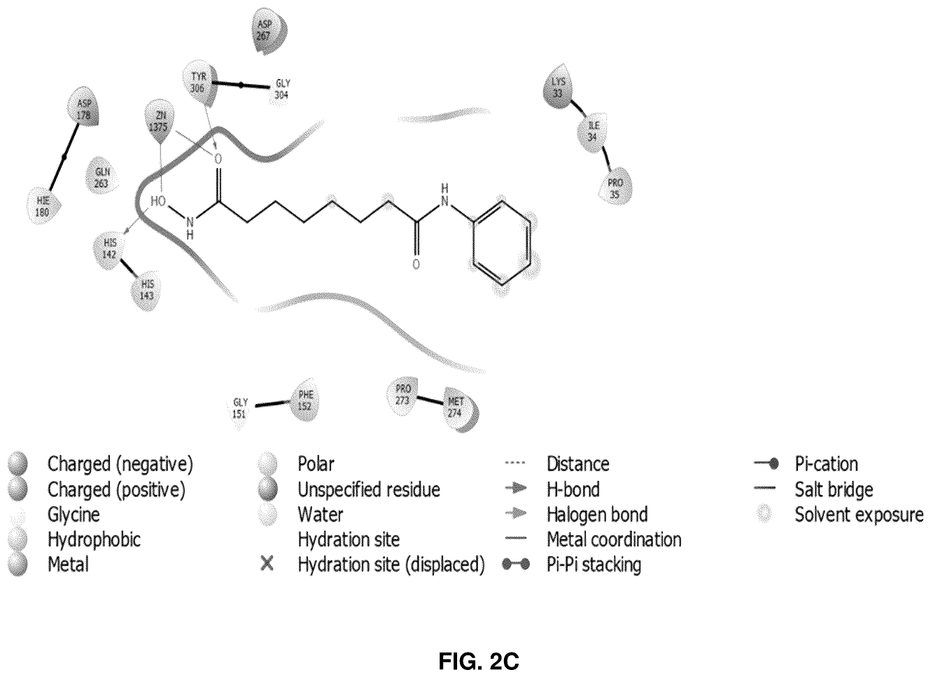

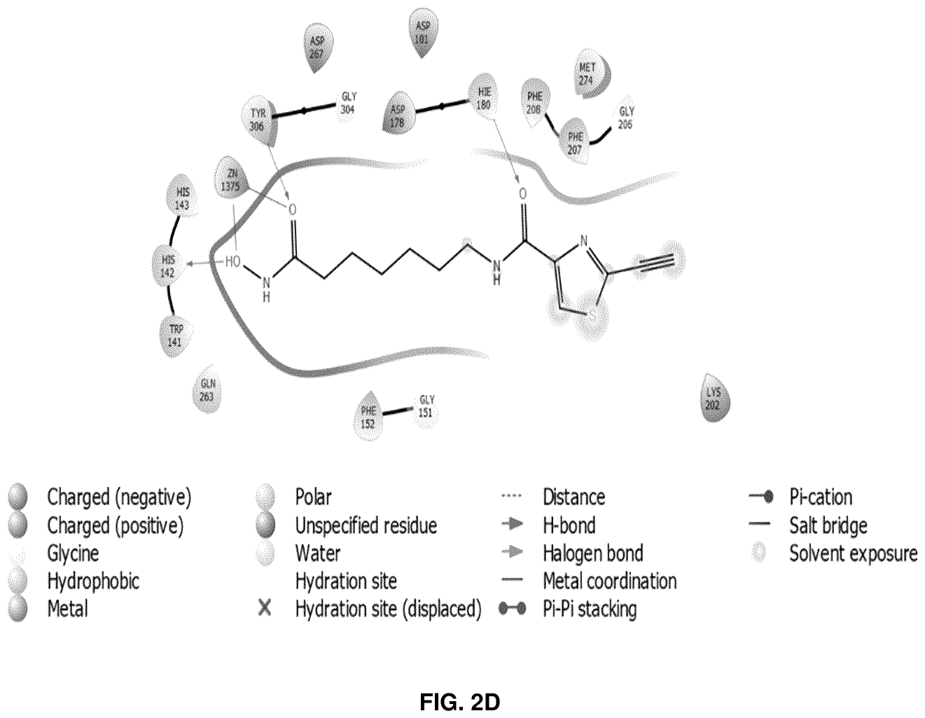

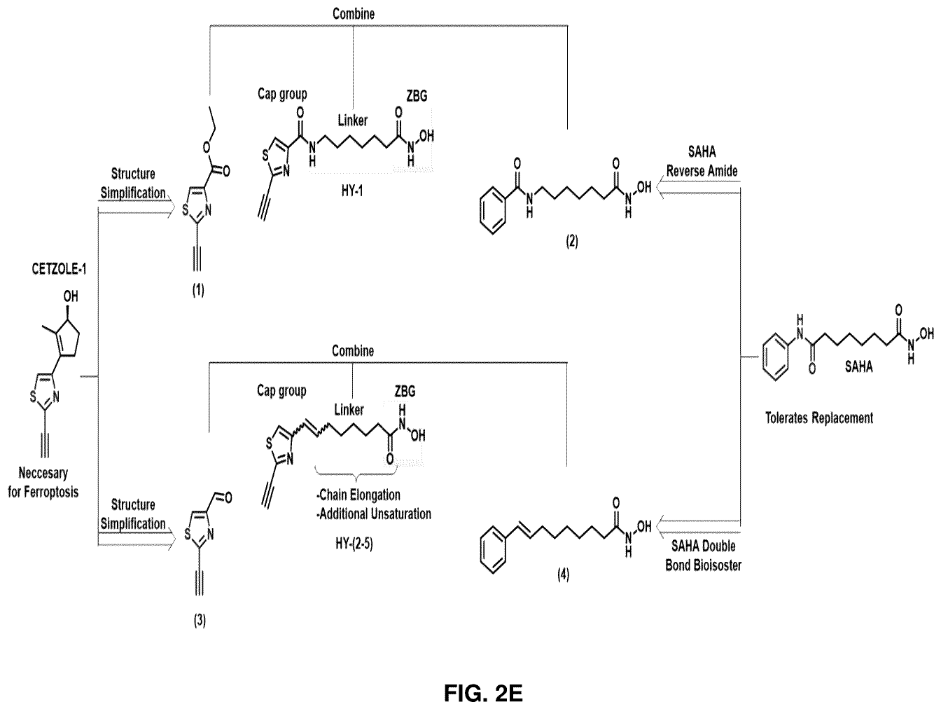

The design of HDACi was dictated by the characteristics of the active site (FIG. 2A). The pan inhibitor SAHA was used for the design of the first generation ferroptosis HDACi hybrids. Hydroxamic acid was chosen as the ZBG group while a short aliphatic linker of comparable length to that of SAHA was used. It was believed that the cap group would be the ideal site for positioning the ferroptotic pharmacophore. Although many different pharmacophores have been reported to be capable of inducing ferroptosis, incorporation of some of them into a hybrid molecule is challenging. For example, erastin has a large pharmacophore with strict structure-activity-relationship (SAR) requirements, thus rendering its incorporation into an HDAC pharmacophore without affecting HDAC and ferroptosis activities challenging. A ferroptotic agent referred to herein as CETZOLE-1 (FIG. 2E), which has a 4-cyclopentenyl-2-ethynylthiazole scaffold (therefore, referred to as a CETZOLE) has been discovered. This compound induces ferroptosis, but has a simpler structure, making its incorporation into hybrid molecules easier. In addition, extensive SAR studies have revealed that the small 2-alkynyl thiazole system retains the ability to induce ferroptosis with wider group tolerance at the 5-position. Two different types of structural modifications were performed: the first via a carboxylate at the 5-position appropriate for amide coupling (FIG. 2A), and the second via a formyl group at this position for Wittig reactions (FIG. 2B). These modifications result in analogs that have a bioisosteric relationship. In addition, the Wittig reaction allows the control of the geometry of the double bond as well as the degree of unsaturation of the linker, allowing for a SAR study to be performed in a quest of more potent and/or selective inhibitors. The analogue synthesis via carboxylate would result in reverse amides of SAHA, which, however, are not believed to have any biological significance as such modifications have previously been reported as “non-critical”. Indeed, preliminary molecular modeling data on HDAC-8 (FIGS. 2A-2D) confirm that reversing the amide bond direction and modifying the cap group do affect the binding ability of the resulting HY-1 molecule, which accesses the active site in a way similar to SAHA (for detailed analysis on docking, see below for in-silico evaluation).

Twenty compounds were designed based on this pharmacophore, as summarized in FIG. 3. Members of group A (FIG. 3A) were designed as inactive analogs that can be used as negative controls in biological studies. They lack a terminal alkyne group at the 2-position of thiazole ring that is necessary for ferroptotic activity and a hydroxamic acid metal-binding group necessary for HDAC inhibitory activity (negative controls (NC)). In addition, molecules in group A serve as convenient synthetic intermediates to access the molecules in the other groups. FIG. 3B contains analogs which are designed to induce only ferroptosis without HDAC inhibitory activity (ferroptosis controls (FC)). In contrast, FIG. 3C has analogs that are designed to have only HDAC inhibitory activity without any ferroptotic effect (HDAC controls (HC)). Hybrid molecules that have both ferroptotic and HDAC inhibitory activity are shown in FIG. 3D (hybrid molecules (HY)). The first row includes SAHA-like analogs, which are similar in shape and length. The second and third rows include the E/Z isomers of bioisosteric SAHA analogs. The fourth and fifth rows contain analogs which were designed to investigate the effect of the chain length as well as the degree of unsaturation of the linker, while maintaining the general concept of “SAHA-like” compounds.

### Chemical Synthesis

The amide analogs were synthesized as shown in Scheme 1 (FIG. 4) using ethyl 2-bromothiazole-4-carboxylate (1) and 7-aminoheptanoic acid (6) as the main building blocks. 2-Bromothiazole-4-carboxylic acid obtained by the hydrolysis of ester (1) and the methyl ester of 7-aminoheptanoic acid were subjected to amide coupling under Steglich conditions to obtain compound (NC-1). It served as the inactive control in biological assays, and also as the precursor for the synthesis of rest of the analogs. Sonogashira coupling of NC-1 with trimethylsilylacetylene resulted in the alkynyl ester (FC-1), which was designed to induce only ferroptosis (ferroptosis control (FC)). Hydrolysis of (NC-1) gave the carboxylate (8), which served as the precursor for the synthesis of hydroxamic acids. Direct conversion of the esters NC-1 and FC-1 to hydroxamic acid proved difficult due to the presence of electrophilic bromine or alkyne on the thiazole ring. Coupling of compound (8) with NH 2 OTHP resulted in the hydroxamic acid derivative (9) in good yields. Sonogashira coupling of (9) with TMS-acetylene proceeded efficiently to give the TMS-alkyne derivative, which underwent desilylation during separation resulting directly in compound (10) in moderate yields. Removal of THP protecting group using stochiometric amount of 1 M HC1 in Me0H resulted in the ferroptosis HDACi hybrid (HY-1).

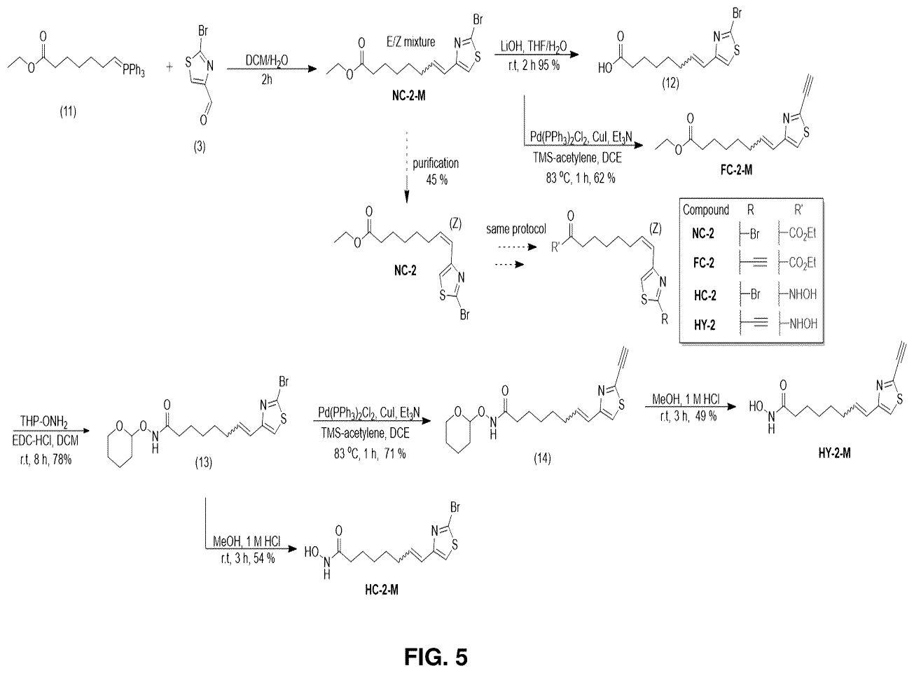

For the synthesis of the non-amide olefinic congeners, a Wittig reaction of 2-bromothiazole-4-carbaldehyde (3) (synthesized in situ) with ethyl 7-(triphenyl-λ⁵-phosphaneylidene)heptanoate (11) resulted in a mixture of both (E) and (Z) isomers NC-2-M (_1:1 E/Z ratio). Slow column chromatography separation on silica allowed the separation of a small amount of pure (Z) analog (NC-2), while the rest was obtained as an E/Z mixture (1:1 ratio) (NC-2-M). Analogues were synthesized using both the pure (Z) analog as well as the E/Z mixture, using similar synthetic procedures (Scheme 2, FIG. 5).

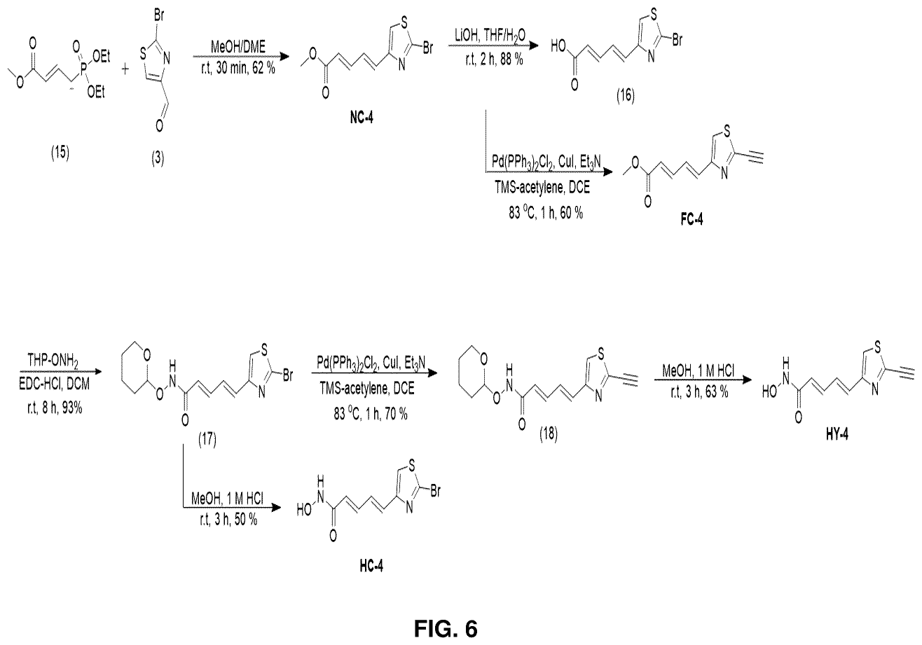

To access the doubly unsaturated linker, the stabilized phosphonate carbanion (15) formed in situ was reacted with 2-bromothiazole-4-carbaldehyde (3) to yield predominantly the E-analog NC-4, followed by a similar synthetic route to access the rest of the analogs of the series (Scheme 3, FIG. 6).

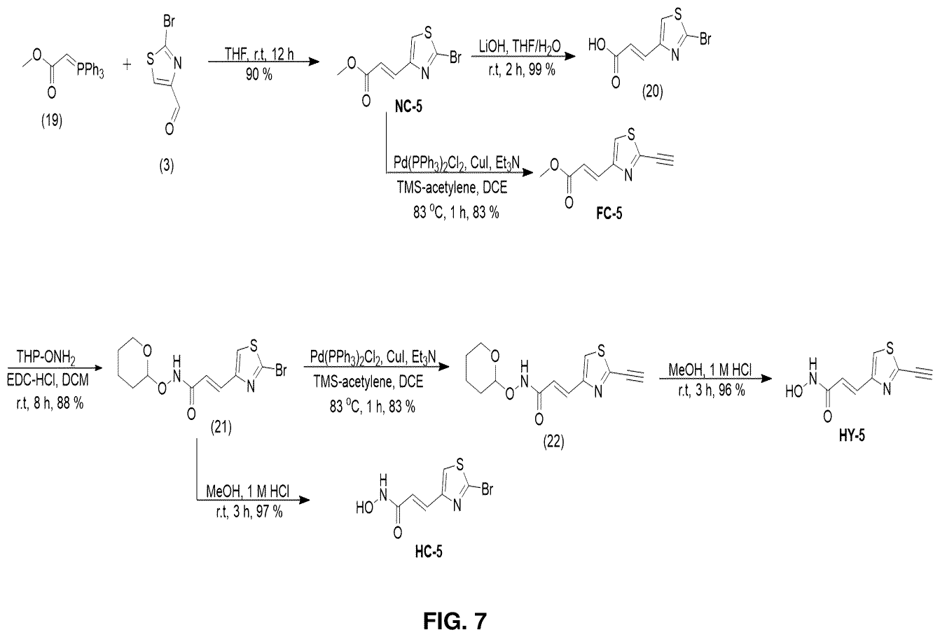

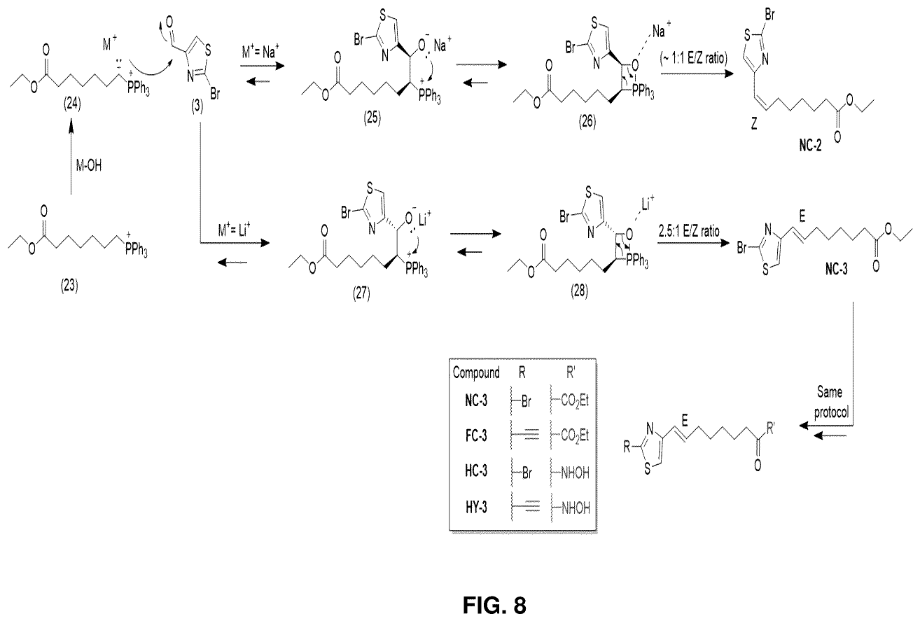

Similarly, reaction of compound (3) with methyl 3-(triphenyl-15-phosphaneylidene)propanoate (19) gave the short unsaturated analogs NC-5, FC-5, HC-5, and HY-5 (Scheme 4, FIG. 7).

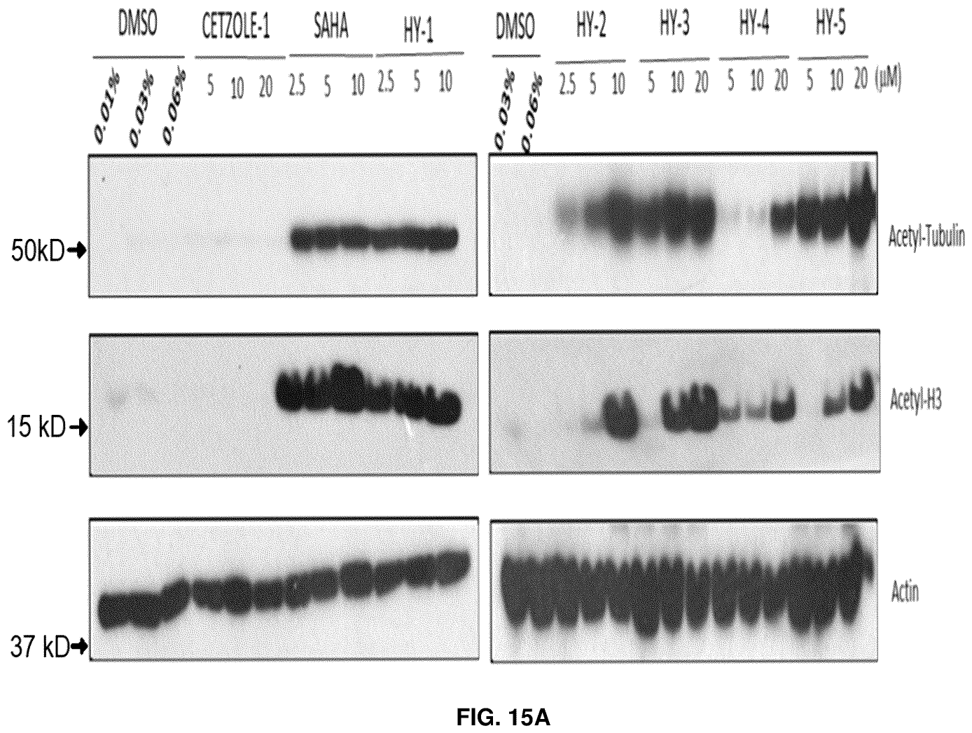

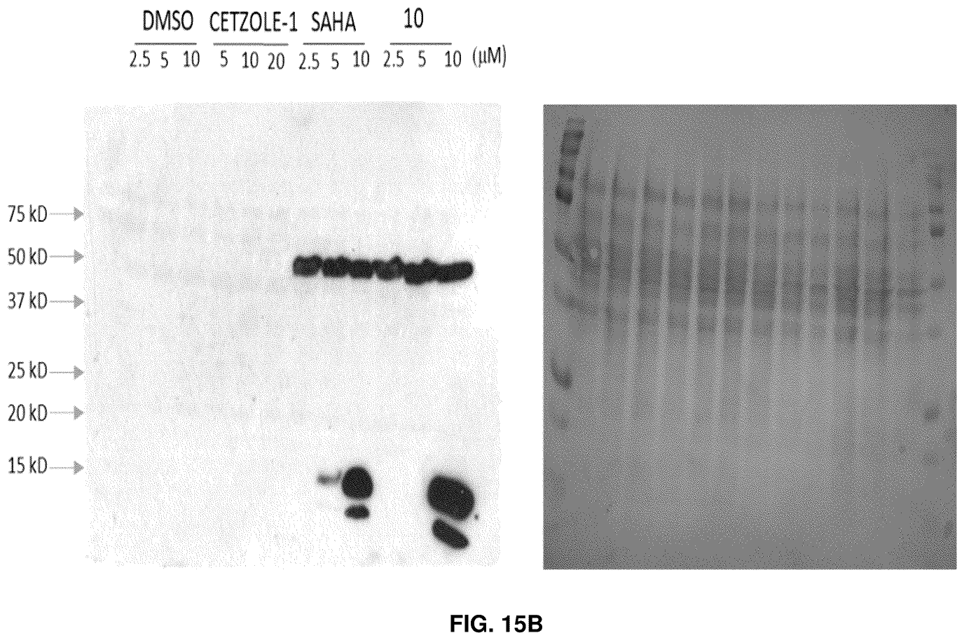

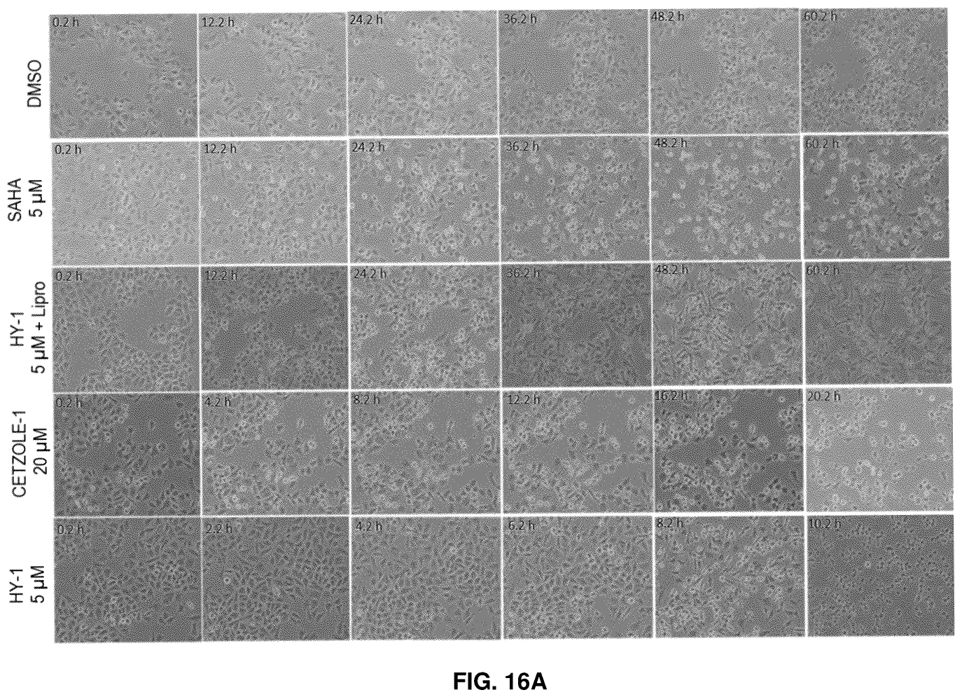

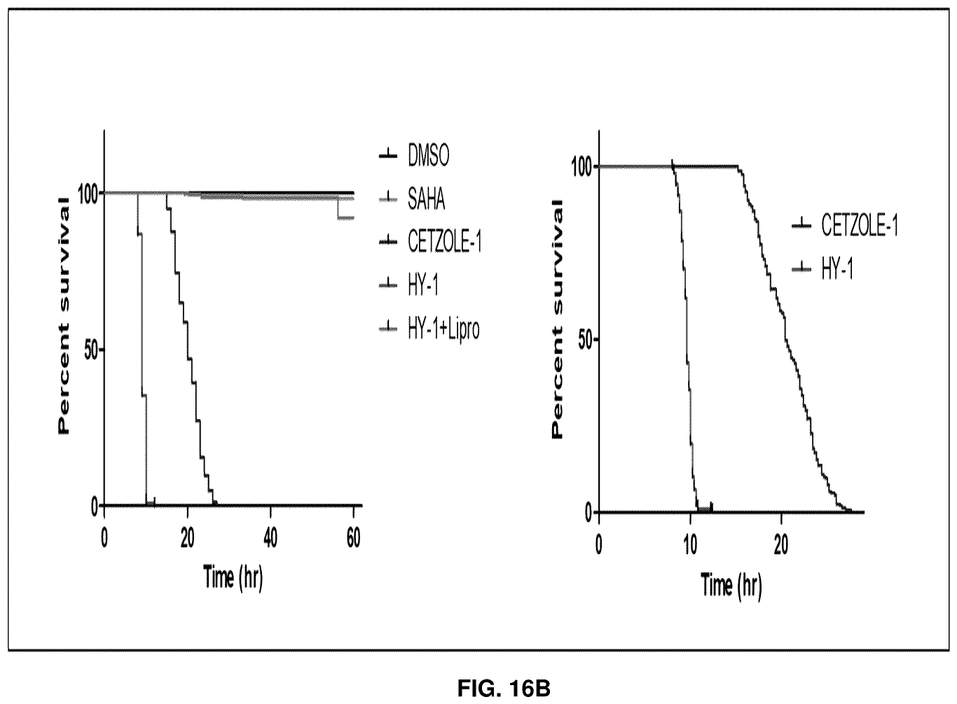

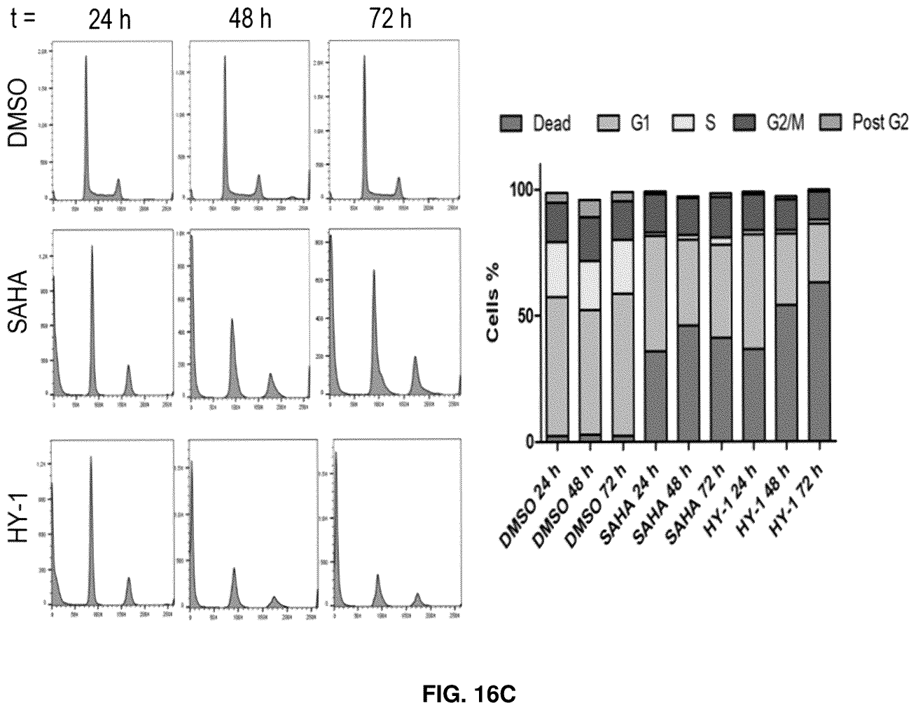

As shown in Table 1, FIG. 15, and FIGS. 16A-16C, there is a difference between the activity profiles of the pure Z-analogs (FC-2, HC-2, and HY-2) and the E/Z-mixtures (FC-2-M, HC-2-M, and HY-2-M). Most E/Z analog mixtures have higher IC₅₀s on both H522 and HCT-116 cell lines. In addition, the E/Z mixture, when tested for H3 and tubulin hyperacetylation, did not show results similar to those of the pure Z-analog. The latter shows H3 hyperacetylation only at 10 mM and tubulin hyperacetylation at 5 mM and 10 mM. In contrast, the E/Z mixture did not show H3 hyperacetylation even at 10 mM, and showed tubulin hyperacetylation only at 10 mM. Without wishing to be bound by theory, it is believed that the different pharmacological profiles stem from different activities of E and Z isomers, with the E-analogs being less reactive than the Z-analogs. To confirm this, pure E-analogs were synthesized.

The Wittig reaction has the inherent tendency to provide mainly the Z-isomer as the faster formed kinetic product. The nature of the counter ion of the base used can have an effect on the stereochemical outcome of the reaction. The smaller and harder Lewis acidic Li+ions result in mostly the E-isomer and softer counter ions such as Na lead to equimolar E/Z mixtures. Indeed, using LiOH as the base resulted in mixtures enriched with E-analogue (2.5:1 E/Z ratio), allowing for its partial separation and purification. Although the Horner—Wadsworth—Emmons reaction would be a good alternative, it was not undertaken due to success in obtaining the E-isomer by counter ion control.

### The Hybrid Molecules are Potent Cytotoxic Agents

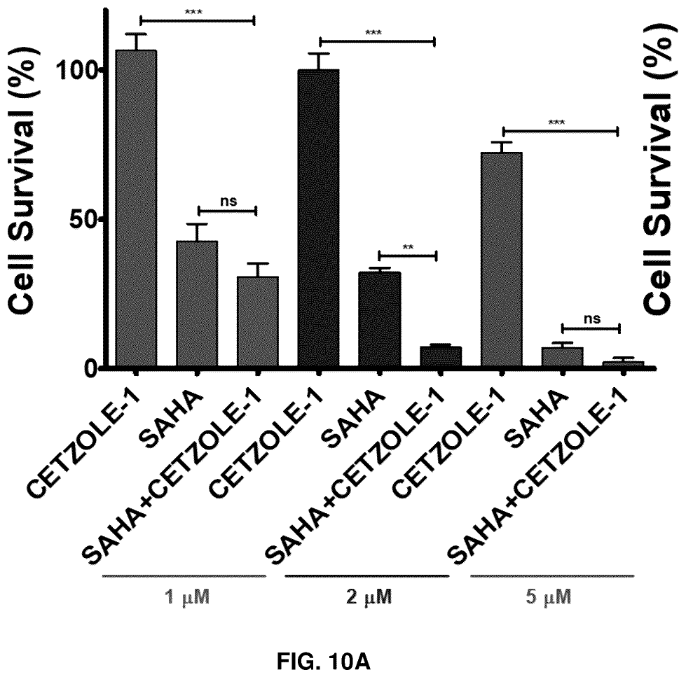

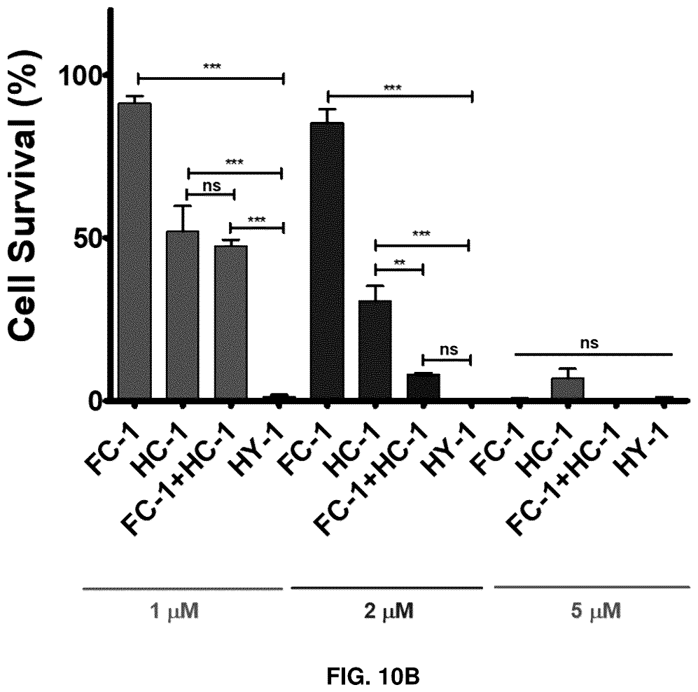

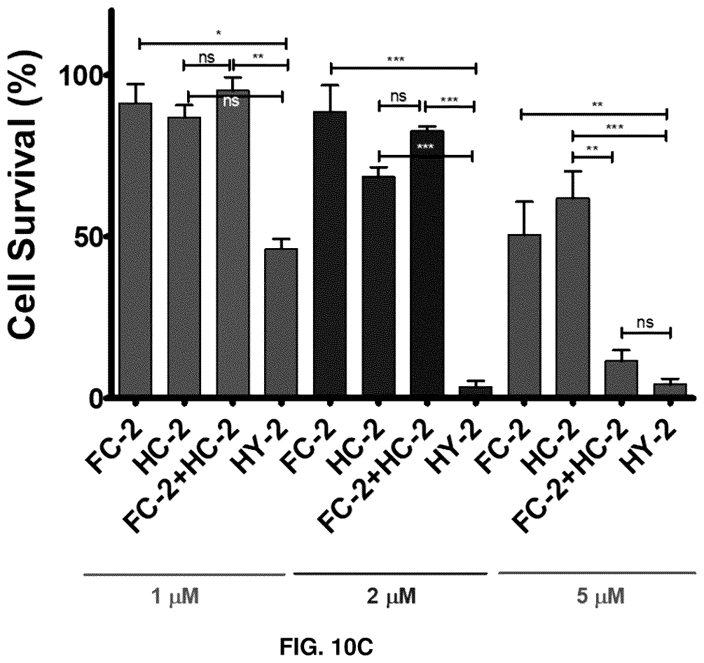

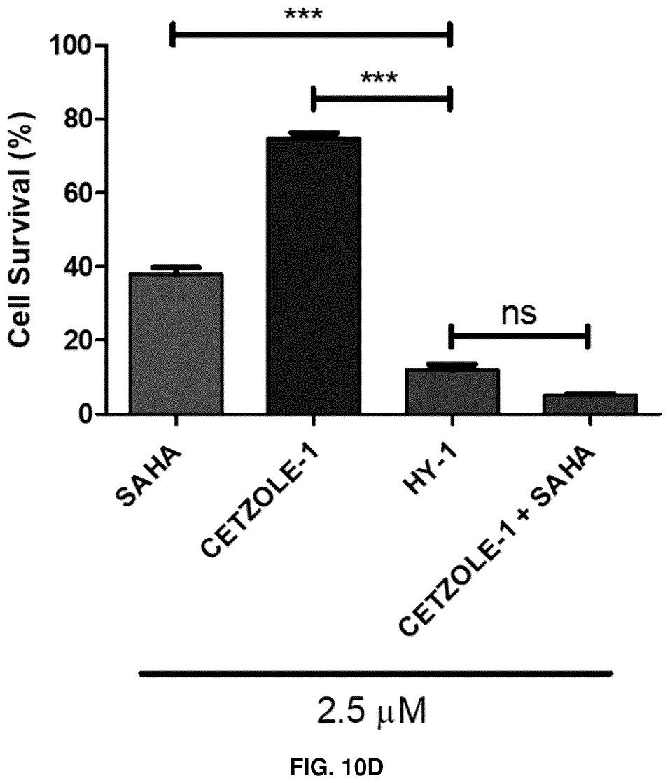

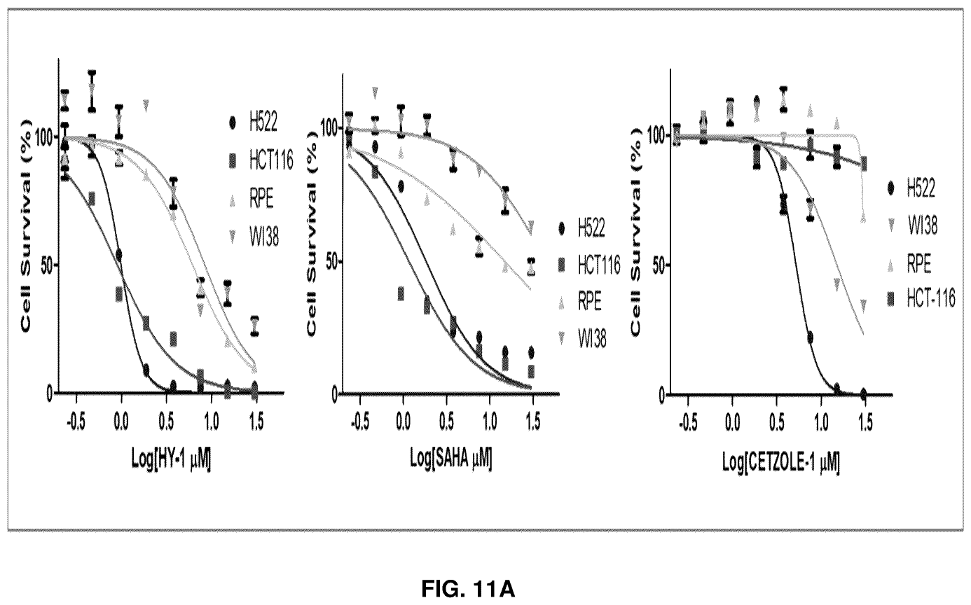

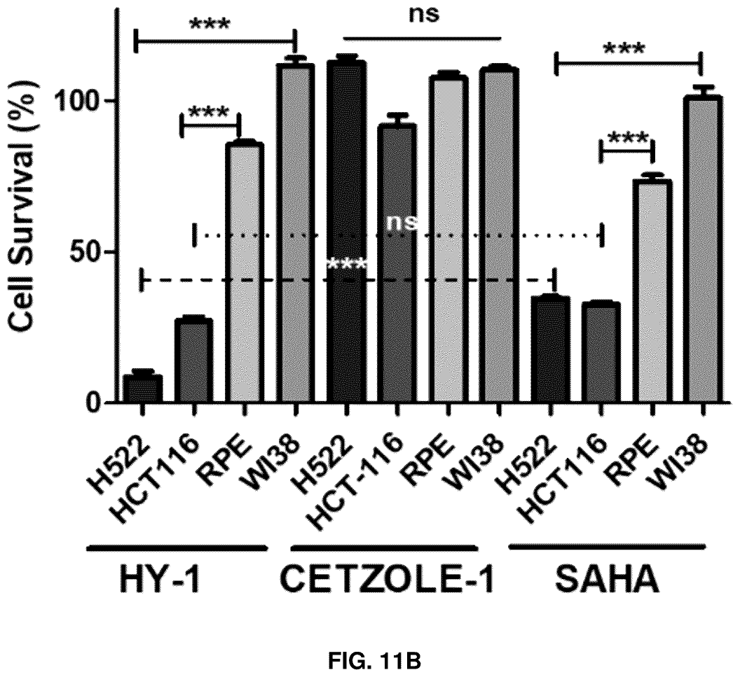

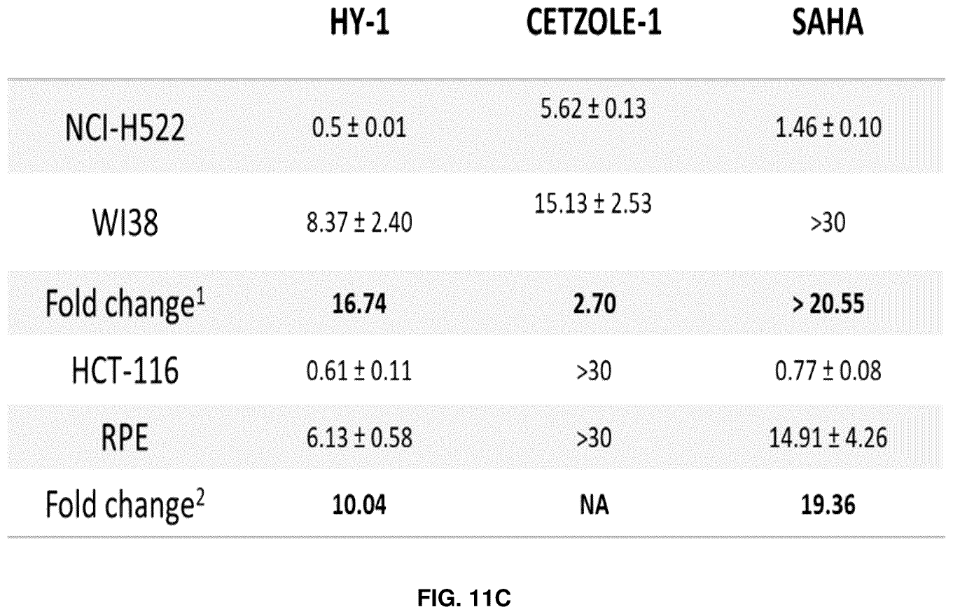

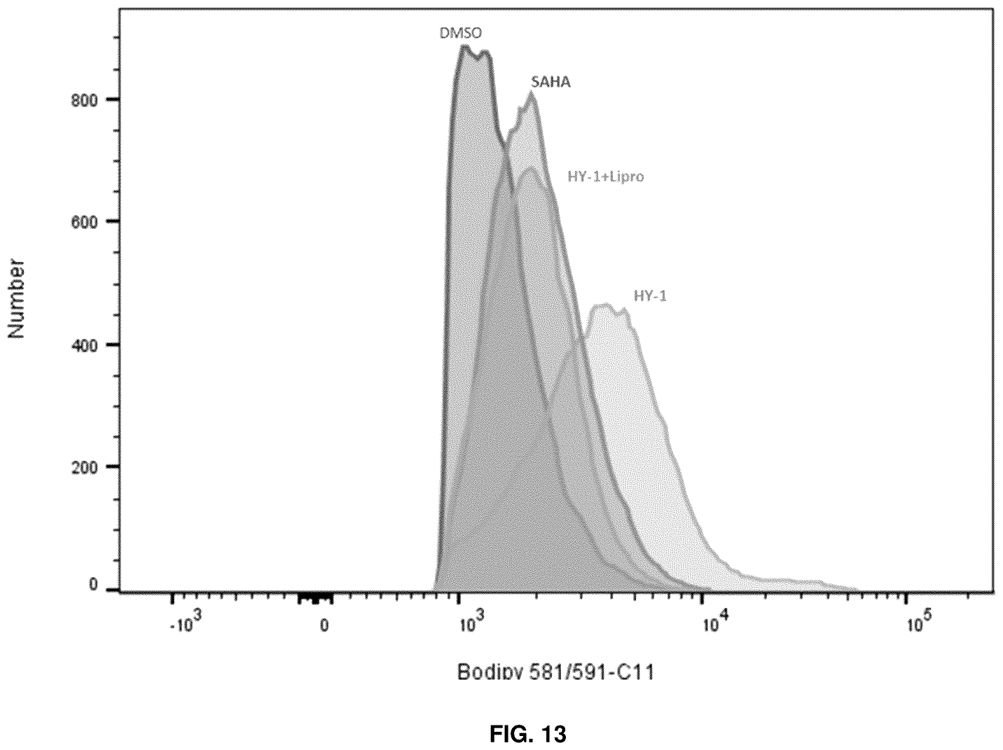

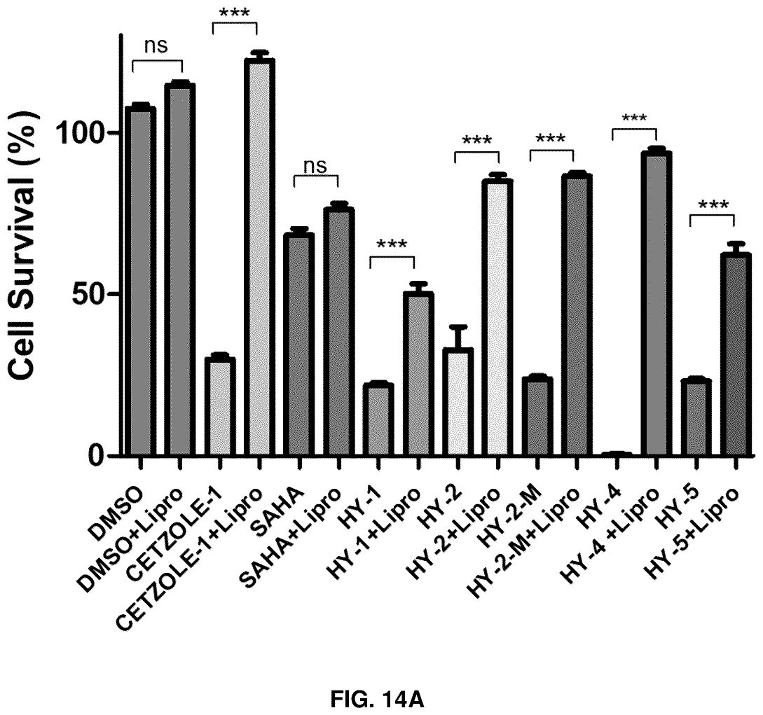

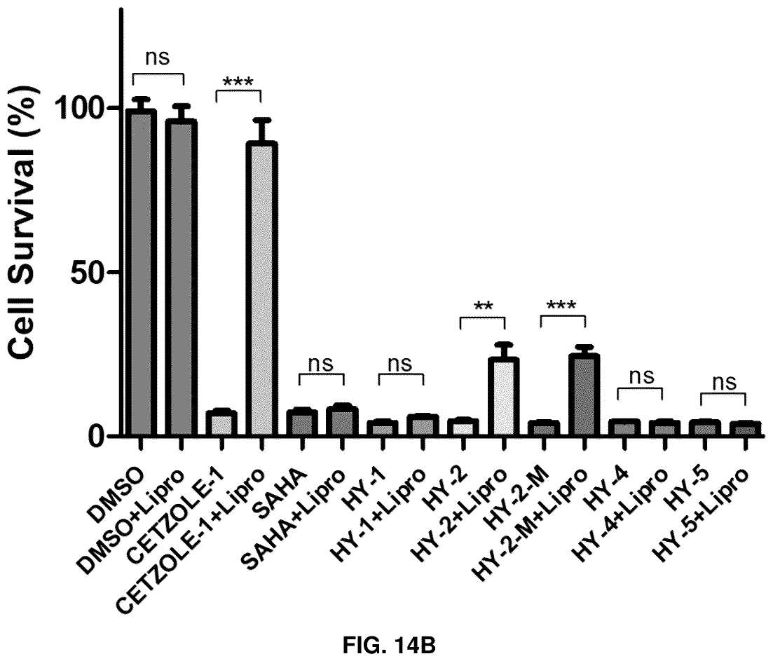

The cytotoxic effects of the compounds was first investigated using two cell lines: NCI-H522 (non-small lung cancer) and HCT116 (human colon carcinoma). Previous studies have shown that NCI-H552 cells undergo ferroptosis, while the HCT116 cells were insensitive to ferroptosis induced by CETZOLEs, sulfasalazine, or simple cystine deprivation. The HCT116 cells are killed by erastin, however, this is only partially explained by ferroptosis, with erastin likely having other targets in this cell line. Comparing effects in NCI-H522 and HCT116 made it possible to delineate the HDAC inhibitory and ferroptotic effects. The negative control (NC) analogs were inactive against both cell lines. The compounds that were designed to induce only ferroptosis (ferroptosis control) showed low mM activity only on the NCI-H522 cell line (2-13 mM IC₅₀), but were inactive against HCT116 cells, except for analog FC-5, which showed some activity on HCT116 (13.73 mM IC₅₀) (Table 1). To investigate if this effect is due to ferroptosis or some other activity, the cells were co-treated with the ferroptosis inhibitor Liproxstatin-1 (0.25 mM), which rescued the cells (FIG. 14A-14B). The HDAC inhibitors showed good activity on both cell lines. The hybrid molecules showed enhanced killing of NCI-H522 cells (0.5-3.61 mM IC₅₀) and HCT116 cell line (0.61-8.67 mM IC₅₀) In fact, at concentrations close to 2.5 mM, most of the hybrid molecules killed NCI-H522 cells to a significantly greater extent than any of the controls used, including CETZOLE-1 and SAHA (FIGS. 11, 13, MS4). This indicates a synergistic or additive effect, because the same phenomenon was not observed with HCT116 cells, where at the same concentrations, the activity of hybrid molecules was similar to that of SAHA (FIGS. 11, 13, MS4). In addition, a similar observation was made on co-treatment of the two types of cells with CETZOLE-1 and SAHA (FIG. 10A). This is in line with synergistic effects from HDAC inhibitors and ferroptosis inducers. The E and Z isomers have different pharmacological behavior with respect to HDAC inhibitory activity, as seen from the activity profiles of both the HDAC controls (bromo-analogs) and the hybrid molecules (Table 1). Because of the different activity profiles in the rest of the biological assays, the pure E-analogs were used further and not the mixture. Although in this case the activity differences are minimal, it is important in medicinal chemistry and drug design to evaluate both activities and side effects of individual isomers and decide whether the convenient choice of using a mixture is appropriate or not.

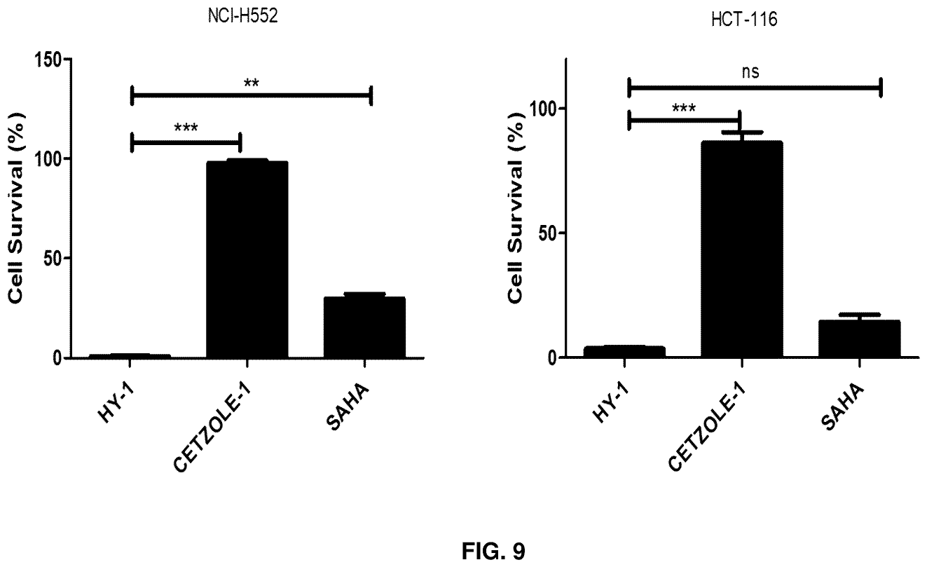

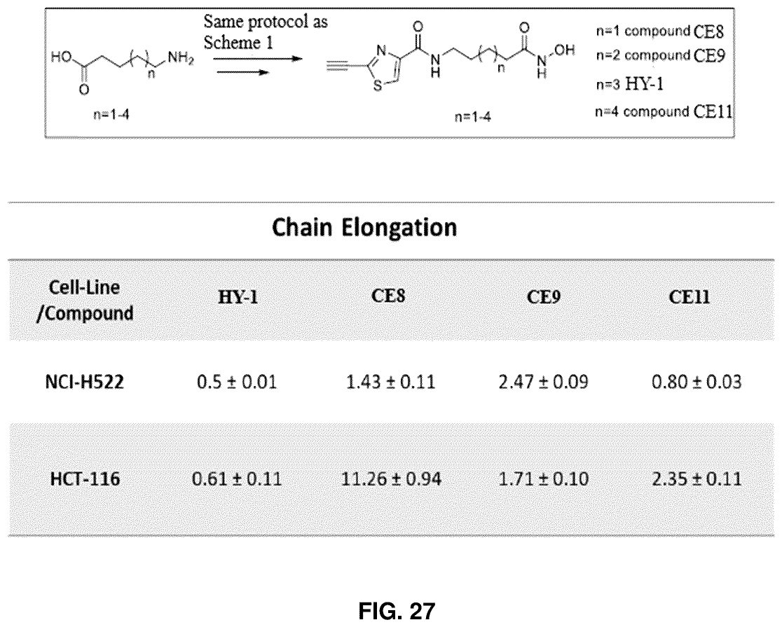

Driven by the demonstrated antiproliferative activity of HY-1, (Table 1 and FIG. 9), a chain elongation study of the linker was performed connecting the ferroptotic cap group with the ZBG (FIG. 27). The results indicate that the initial design was ideal because HY-1 demonstrated a superior cytotoxicity profile compared to its shorter or longer analogs. Thus, HY-1 was used in most of the biological assays as it was the most potent analog.

TABLE 1
IC50 ± SD (n = 3) values (mM) for the designed library of compounds
on NCI-H522 and HCT-116 cell lines.
Hybrid Molecules

| Cell- | Compound | | | | | | |
|---|---|---|---|---|---|---|---|
| Line | HY-1 | HY-2 | HY-2-M | HY-3 | HY-4 | HY-5 | N/A |
| NCI- | 0.5 ± | 1.07 ± | 1.67 ± | 1.59 ± | 0.52 ± | 0.79 ± | N/A |
| H522 | 0.01 | 0.10 | 0.06 | 0.16 | 0.04 | 0.04 | |
| HCT- | 0.61 ± | 1.19 ± | 3.42 ± | 4.96 ± | 5.43 ± | 5.16 ± | N/A |
| 116 | 0.11 | 0.07 | 0.47 | 0.78 | 1.34 | 0.76 | |

HDACi

| Cell- | Compound | | | | | | |
|---|---|---|---|---|---|---|---|
| Line | HC-1 | HC-2 | HC-2-M | HC-3 | HC-4 | HC-5 | SAHA |
| NCI- | 3.26 ± | 8.94 ± | 9.54 ± | 20.71 ± | 12.48 ± | 3.23 ± | 1.46 ± 0.10 |
| H522 | 0.26 | 2.18 | 2.00 | 3.27 | 1.07 | 0.25 | |
| HCT- | 1.09 ± | 6.28 ± | 11.99 ± | 12.95 ± | 17.20 ± | 1.30 ± | 0.77 ± 0.08 |
| 116 | 0.12 | 0.86 | 3.42 | 1.95 | 2.95 | 0.14 | |

Inducers of Ferroptosis

| Cell- | Compound | | | | | | |
|---|---|---|---|---|---|---|---|
| Line | FC-1 | FC-2 | FC-2-M | FC-3 | FC-4 | FC-5 | CETZOLE-1 |
| NCI- | 3.33 ± | 7.33 ± | 13.13 ± | 15.7 ± | 2.80 ± | 2.29 ± | 5.62 ± 0.13 |
| H522 | 0.15 | 1.28 | 2.00 | 4.51 | 0.34 | 0.36 | |
| HCT- | >40 | >40 | >40 | 33.01 ± | >40 | 13.73 ± | >40 |
| 116 | | | | 1.42 | | 4.64 | |

Inactive analogs
For all inactive analogs IC₅₀ > 40 mM for both Cell Lines
N/A: not applicable.

In FIG. 9, the ability of the hybrid molecules to show enhanced cytotoxicity on the ferroptosis sensitive NCI-H522 cell line is demonstrated. In order to investigate how this enhancement compares with treatment with equimolar combinations of appropriate ferroptosis and HDAC controls, cell survival assays were performed at concentrations of 1 μM, 2 μM, and 5 μM for CETZOLE-1, SAHA, and their equimolar combinations (FIG. 10A), as well as the two most active hybrid molecules, HY-1 (FIG. 10B) and HY-2 (FIG. 10C), and equimolar combinations of the two individual components. The results indicate that hybrid molecules demonstrate enhanced cytotoxicity (preferably at concentrations around 2 μM) in a similar way to the equimolar combination of the two individual components, as in the case of HY-1 (FIG. 10B). But if the corresponding controls are significantly less potent than the hybrid molecules, such as in the case of HY-2, then the equimolar combination requires higher concentrations (5 μM) to reach cytotoxicity levels comparable to that of the hybrid molecules, as seen in FIG. 10C. Similar results were observed for HY-1 with MDA-MB-231, which is another ferroptosis sensitive cell line (FIG. 10D).

A salient property of a successful chemotherapeutic agent is the ability to discriminate between normal cells and cancer cells. Lack of selectivity is usually the cause of severe side effects. To investigate the ability of the hybrid molecules to selectively kill cancer cells over normal cells, the cytotoxicity of one of the most potent hybrid analog, HY-1, was tested on WI-38 cells (normal human lung fibroblasts) and hTERT-immortalized RPE cells (retinal pigment epithelial cells) (FIGS. 11A-11C). The two parent molecules SAHA and CETZOLE-1 were used as controls. Based on the mean IC₅₀ values, the hybrid molecule was approximately 17-fold more selective for H522 over WI-38 and 10-fold more selective for epithelial HCT116 over RPE cell lines (FIGS. 11A, 11C). The control CETZOLE-1 did not show any notable effect on both cancerous HCT116 and normal RPE epithelial cells (IC₅₀>30 μM) (FIGS. 11A, 11C). However, it showed poor selectivity (approximetly 3-fold) for NCI-H522 cells over the WI-38 (FIGS. 11A, 11C). This can be explained as being due to fast and stress-induced cell death, which is an inherent characteristic of ferroptosis. In contrast, SAHA kills cells with more than 20-fold selectivity for NCI-H522 over WI-38 and 19-fold selectivity for HCT116 over RPE. It is noteworthy that the hybrid molecule which combines characteristics from both mechanisms has selectivity rates in-between those of ferroptosis inducer and HDACi. Interestingly, comparison of selectivity at concentrations of therapeutic relevance (2 μM) shows an enhanced cytotoxic effect of the hybrid molecule on the NCI-H522 cell line in comparison to SAHA and CETZOLE-1, but at the same time the survival rate of WI-38 is comparable to that of SAHA (FIG. 11B). Thus, the hybrid molecule in the mesenchymal system has a significantly greater cytotoxic effect on cancer cells compared to SAHA and CETZOLE-1, while at the same time showing no toxic effects on normal cells. On the epithelial system, the sensitivity to hybrid molecule is comparable to that of SAHA with respect to killing of the cancer cells (HCT116), while no toxicity was observed on the normal cells (RPE)(FIG. 11B).

Neuronal cells are inherently more sensitive to ferroptosis, as they have higher levels of polyunsaturated fatty acids (PUFAs), which serve as precursors for lipid peroxidation. In addition, due to their high metabolic activity, brain cells are particularly vulnerable to oxidative stress. Indeed, ferroptosis plays an important role in a series of neurodegenerative diseases, and CNS-related toxic effects can explain the failure of ferroptotic agents in clinical applications as potent anticancer agents. Thus, there is a need to find strategies to attenuate their neurotoxic effects while retaining the beneficial anticancer effects. Most ferroptosis inhibitors are capable of inhibiting ferroptosis in both neuronal and cancer cells, but class I histone deacetylase (HDAC) inhibitors selectively protect neurons, while enhancing ferroptosis in cancer cells. Although the pathways that lead to ferroptosis-mediated neuronal and cancer cell death are the same, they are differently regulated by HDACs. Thus, the hybrid molecule HY-1 and its corresponding negative controls were tested on the SH-SYSY cell line (human neuroblastoma), which can serve as a model for neurodegenerative diseases. As shown in FIG. 12A, the inactive analog NC-1 had no effect on the neuronal cells. The ferroptosis inducer FC-1 showed significant neurotoxicity. Interestingly, the HDACi HC-1 showed very mild toxicity on the neuronal cells at the highest concentration tested. Strikingly, the hybrid molecule HY-1, when tested at concentrations of therapeutic relevance (2.32 μM), had no neurotoxic effects. These results are in line with the belief that HDACi may attenuate ferroptotic neurotoxicity. The latter findings become more important when the effects on neuronal cells are contrasted with the effect on the cancer cell lines NCI-H522 and HCT116 (FIG. 12B), where opposite effects are observed. In the NCI-H522 cell line, which is sensitive to both ferroptosis and HDACi, the hybrid molecule showed significant effect compared to its ferroptosis or HDACi only controls. Since HCT116 cells are relatively resistant to ferroptotic cell death, the observed sensitivity is mostly due to HDAC inhibition. Thus, the sensitivity to hybrids and the HDACi controls are of comparable rates. (The same applies for CETZOLE-1 and SAHA, see FIG. 9). The analog HY-4 (and its corresponding controls) did not show enhanced killing of the H522 cell line, while at the same time not showing attenuated neurotoxicity, indicating the possibility that the mechanisms responsible for both effects are the same. Without wishing to be bound by theory, it is believed that the failure of this analog to provide beneficial effects is due to its poor HDAC inhibitory activity, as indicated by its relatively high HCT116 IC₅₀ values (Table 1), low levels of H3 and tubulin hyperacetylation (FIG. 15), and the HDAC isoform selectivity data (FIG. 17). As a result, its ferroptotic activity predominates, indicating that a balance between ferroptosis and HDAC inhibition activities is required. A similar result with HY-4 was obtained with PC-12 (pheochromocytoma) cells as well. Non-differentiated PC-12 cells have stem like properties, but when differentiated by nerve growth factor, they demonstrate neuronal behavior (FIG. 12C). Both these states proved to be resistant to the ferroptosis control (FC-1), indicating resistance to ferroptosis induction. As a consequence, it was not possible to test the protective effect of the HDAC moiety on ferroptosis in this neuronal cell line. In fact, the hybrid HY-1 behaved like the HDAC control HC-1 (FIG. 12D). These results indicate that the hybrid molecules have attenuated neuronal toxicity in a cell line dependent manner Molecular crosstalk between HDACi and ferroptosis may be responsible.

### The Hybrid Molecules Induce Ferroptosis while Maintaining HDAC Inhibitory Activity

Next, key biomarkers were investigated to directly assess ferroptosis and HDAC inhibition by the hybrid molecules. To measure ferroptosis, lipid peroxidation was first investigated. The fluorescent dye C11-BODIPY^{581/591} is an established system for the quantification of oxidation processes in membranes of living cells that lead to accumulation of lipid peroxides. Fluorescent measurements in the green area of the spectra can provide information on the levels of the oxidized form of the dye, which are proportional to the lipid peroxide levels. Liproxstatin-1 has previously been identified as a potent small molecule that suppresses ferroptosis in cells and inhibits lipid peroxidation by acting as a radical trapping antioxidant selectively on lipid bilayers. Flow cytometry data (FIG. 13) shows that treatment of NCI-H522 cells with the hybrid molecule (HY-1) leads to increased levels of lipid peroxides within 6 hours of treatment. When HY-1 was used in combination with Lipoxstatin-1, lipid peroxidation levels decreased. It was a perfect overlap with the graph obtained with SAHA treatment. HDACis have been reported to increase reactive oxygen species (ROS) production. In one previous study, SAHA resistant cell lines failed to show accumulation of ROS, in contrast to significant increases in ROS levels in sensitive cell lines, in caspase independent manner Normal cells exhibited increased levels of the antioxidant protein thioredoxin, thereby revealing a possible mechanism for tumor cell selectivity of HDAC inhibitors. Thus, the increased fluorescence in cells treated with SAHA or HY-1+Liproxstatin-1 compared to DMSO may stem from this effect. Increased levels of ROS may serve as one of the possible origins of synergism between HDAC inhibitors and ferroptosis inducers. To investigate whether lipid peroxide accumulation is the only driving force that leads to cell death, rescue experiments were performed in which NCI-H522 cancer cells were treated with several of the inhibitors in the presence and absence of Liproxstatin-1 for either one day or four days (FIGS. 14A-14B). Lirpoxstatin-1 showed significant rescue from ferroptosis inducer CETZOLE-1 and the hybrid molecules, while cell death from SAHA after one-day treatment was only about 30%. However, although Lirpoxstatin-1 still rescued cells from CETZOLE-1 after four days of treatment, it failed to do so with the hybrid molecules due to activation of non-ferroptotic cell death pathways arising from the potential HDAC activity of the hybrid molecules.

Next, whether the hybrid molecules inhibit HDAC proteins was investigated by measuring hyperacetylation of the HDAC substrates, histones for class I inhibition and tubulin for class II (HDAC-6) (FIG. 15A). CETZOLE-1 did not increase the acetylation levels of either H3 or tubulin, while the hybrids HY-1 and HY-2 led to hyperacetylation of both in a similar way to SAHA (pan-inhibitor). Interestingly, compound HY-5, at the a concentration of 5 μM, did not show any H3 hyperacetylation, but only tubulin hyperacetylation, indicating isoform selectivity. Compound HY-4 did not show significant effects at the tested concentrations.

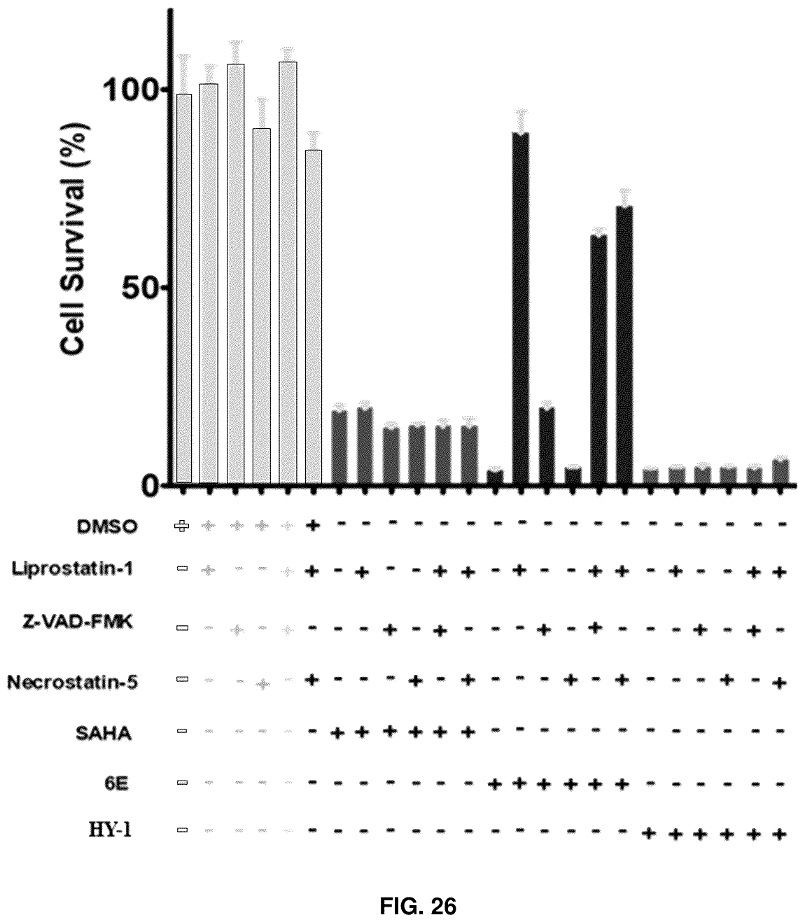

Activation of the ferroptotic pathway by hybrid molecules was also indicated by increased levels of transferrin receptor, which has been identified as a selective marker for ferroptosis. Hybrid molecules also increased caspase-3 cleavage, indicating activation of the apoptotic pathway, possibly as a result of HDAC inhibition. However, the pan-caspase inhibitor Z-VAD-FMK did not rescue cells from HDACi SAHA and had no effect on hybrid-induced death (FIG. 26). This observation can be attributed to additional non-caspase-mediated cell death induced by HDACi. In addition, other cell death inhibitors like autophagy inhibitors had no effect on hybrid-induced cell death (FIG. 26). Thus, hybrid molecules increase the pleiotropic effects of HDAC inhibitors with ferroptosis providing an additional mechanism of action as a means to overcome drug resistance. Another important characteristic of ferroptosis is that cell death is fast, possibly due to self-amplifying oxidation reactions.

In contrast, HDAC inhibition has a slower mechanism of action, which through epigenetic regulation of histones, or hyper-acetylation of other proteins, arrests cells in G1 and/or G2/M, eventually leading to cell death. The observations in these examples with SAHA, CETZOLE-1, and the hybrid molecule HY-1 were consistent with these ideas (FIG. 16A). NCI-H522 cells treated with SAHA were alive even 60 hours after treatment. In contrast, CETZOLE-1-induced ferroptosis began approximately 12 hours after treatment, and within the next 8 hours most of the cells were dead. Strikingly, the hybrid molecule induced death even faster than CETZOLE-1, and the wave-like propagation of ferroptosis is more intense. After initiation of ferroptosis 8 hours after treatment, all cells were dead in the next 2 hours. However, in the presence of Liproxstatin-1, the hybrid molecule behaved more like SAHA, with cells still alive even 60 hours after treatment. Next, cells were co-treated with Liproxstatin-1 and HY-1 to see the prolonged effect of HDACi on cell cycle arrest as determined by flow cytometry of cells stained with propidium iodide (PI) (FIG. 16C). NCI-H522 cells treated with DMSO and Liproxstatin-1 have a distribution of cells between G1, S, and G2/M. However, when treated with SAHA and the hybrid molecules (in combination with Liproxstatin-1) at different time points, the S population of cells disappeared and the majority of cells were either dead or possibly arrested in the G1 or G2/M phase. The flow cytometry data shows once again that if the ferroptotic activity of the hybrid molecules is diminished by the addition of a ferroptosis inhibitor like Liproxstatin-1, they behave in a similar way to SAHA, indicating their ability to inhibit HDAC proteins (similar results were obtained from combinatorial treatment of SAHA and CETZOLE-1).

### In silico Evaluation

Molecular docking studies of the designed analogs were performed to estimate their potential to bind to HDAC proteins. The docking study was focused on class I and class II HADCs using Maestro v10.6 computational software. Initially, a library of 22 compounds, including the 20 analogs shown in FIG. 3, and CETZOLE-1 and SAHA as controls, was generated. X-ray crystallographic data of the HDAC-isoforms co-crystalized with appropriate inhibitors downloaded from the PDB were used. Rotation of side chains was allowed only for the amino acids within the active site. Extra precision docking of the compounds to selected HDAC-isoforms provided the docking scores of each compound as summarized in FIG. 17.

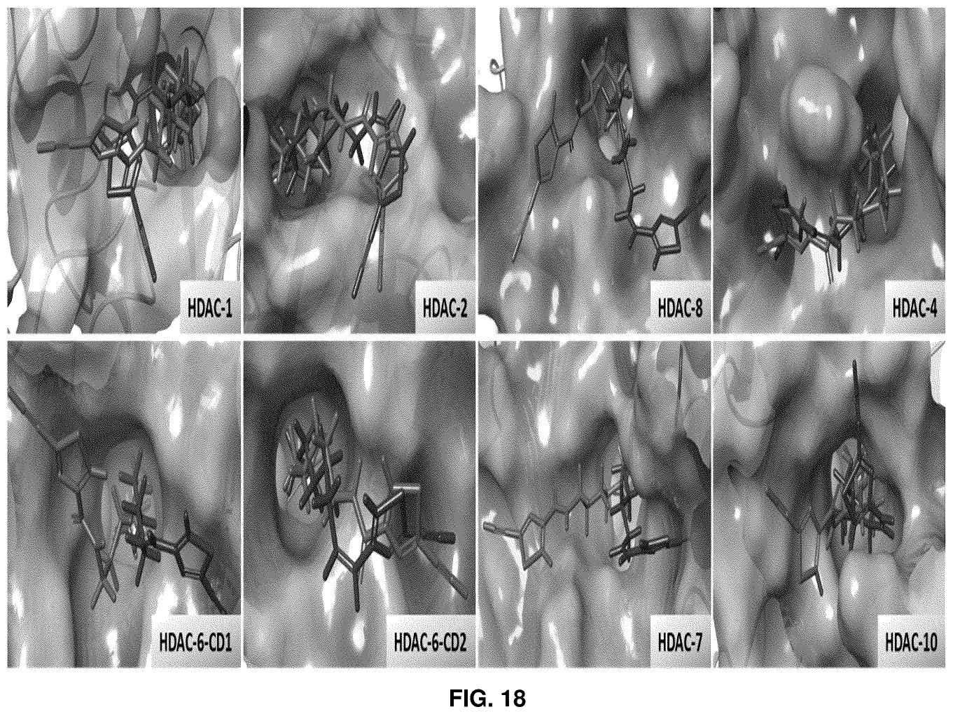

The docking scores indicated that the hybrid molecules as well as the HDACi controls have the ability to bind to the HDAC enzymes in a similar way as SAHA (FIGS. 2, 17). In addition, a slight preference for the class I proteins was observed when compared with class II. An exception to this observation is HDAC-6, for which the hybrid analogs had good docking scores for both catalytic domains (CD1 and CD2). In contrast, analogs that were designed as inactive controls or only as ferroptotic agents, showed poor binding scores, although they have similar shape and form. This is because they lack the ZBG. Pose analysis of binding (FIG. 18 for HY-1 (red) and HY-2 (blue)) reveals that the hybrid analogs retain the ability to bind in a similar binding motif like SAHA (as previously shown in FIG. 2B). The hydroxamic acid chelates with the zinc ion by positioning the linker in the narrow tunnel and the thiazole cap group in the rim area of the HDAC proteins. The surface representation is provided in order to emphasize the complementary shape of the hybrids to the active site (“key into lock”).

### NCI-60 One Dose and Five Dose Assays

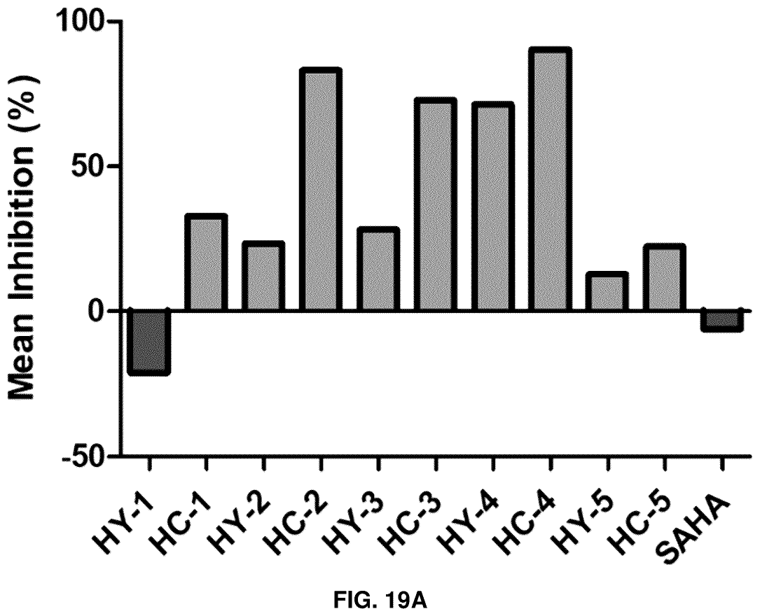

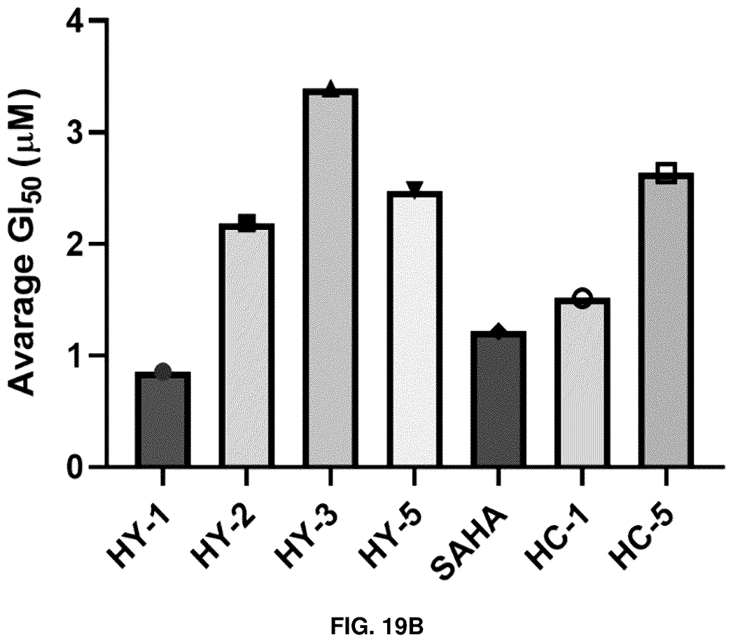

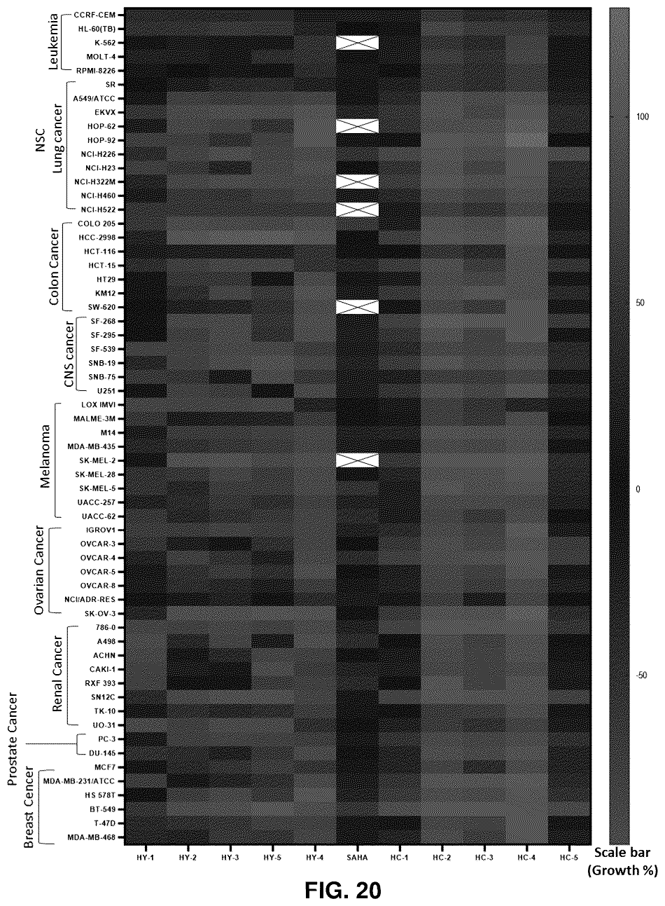

The cytotoxicity of the hybrid analogs was additionally evaluated in the NCI-60 human tumor cell lines screen by the National Cancer Institute (NCI) through the development therapeutics program (DTP), initially at a single dose of 10 mM to determine mean inhibition (FIG. 20) and then in a five dose assay to determine growth inhibition (GI₅₀) values (FIG. 21). The results for both one dose and five dose assays are summarized in FIGS. 19A-19B where the mean values from approximately sixty cell lines are included. Because the compounds which included only the ferroptosis component had poor activity profiles in the NCI 60 cell line assay, the ferroptosis positive controls were not tested in this assay. Consistent with performed cytotoxicity evaluations, data obtained by the NCI-60 DTP confirmed that HY-1 demonstrated the best cytotoxicity profile when compared to other hybrids or HDAC controls and was even more active than SAHA. Many cell lines across multiple cancer types showed enhanced sensitivity to HY-1 (FIGS. 19A-19B). Taking into consideration that the HDAC inhibitory abilities of HY-1 and SAHA are comparable, the higher cytotoxic activity of HY-1 was attributed to the combination of HDAC inhibition with ferroptosis. For the leukemia cell lines, HY-1 showed an enhanced effect on all tested cell lines with the largest difference being observed with the CCRF-CEM cell line (Growth %: 18.40 for SAHA and −41.42 for HY-1). Similarly, HY-1 showed enhanced effects on the NSC-Lung cancer cell lines, with the most noticeable difference being observed with the NCI-H23 cell line (Growth % 3.30 for SAHA and −74.60 for HY-1). HY-1 and SAHA demonstrated similar activity profiles with the colon cancer cell lines, indicating poor contribution by ferroptosis in this type of cancer. SF-539 and SNB-75 are two CNS cancer cell lines with the most noticeable enhancement of HY-1-induced cytotoxicity (SAHA Growth % 16.80 for SF-539 and 18.00 for SNB-75, HY-1 −80.14 for SF-539, and −65.15 for SNB-75). With respect to melanoma cells, HY-1 has substantially greater activity on LOX IMVI cell line (Growth % −84.63) when compared to SAHA (Growth % −3.4). The same effect was observed with the ovarian cancer cell line IGROV-1 (Growth % 23.7 for SAHA and −72.55 for HY-1). Strikingly, most renal cancer cells were outstandingly more sensitive to all hybrid molecules, when compared to their HDAC controls or SAHA, indicating greater therapeutic benefits arising from the combination of ferroptosis and HDAC inhibition on this type of cancer cells. Of the two prostate cancer cells tested, PC-3 cells showed similar levels of sensitivity HY-1 and SAHA (Growth % 10.4 for SAHA and 14.28 for HY-1), but DU-145 cells were much more sensitive to HY-1 (Growth % 3.5 for SAHA and −51.12 for HY-1). Finally, the robust triple-negative breast cancer (TNBC) cell line MDA-MB-231 showed enhanced sensitivity to HY-1 (Growth % −71.82%) when compared to SAHA (Growth % 16.4). This is of particular importance because TNBC represents approximately 10-15% of all breast cancers and patients with TNBC have a poor outcome when compared to the other subtypes of breast cancer. Additional testing on this cell line further confirmed these results (FIG. 5). In contrast to HY-1, analog HY-4 demonstrated the worst cytotoxicity profile. This analog is a poor HDAC inhibitor and its ferroptosis activity predominates. The other hybrid compounds (HY-2, 3, and 5) demonstrated cytotoxicity profiles in between those of HY-1 and HY-4. Most of the HDAC controls demonstrated activity profiles similar to that of SAHA. This is of particular importance because the HDAC inhibitory ability of hybrid analogs and HDAC controls are similar. Thus, any difference in cytotoxicity stems from the combination of ferroptosis with HDAC inhibition.

Analogs that demonstrated promising cytotoxicity in the one dose assay were tested in the 5-dose assay to determine the GI₅₀ values (FIG. 21). These included all hybrid molecules (except HY-4), HC-1, and HC-5. HY-1 showed the best cytotoxicity profile with an average GI₅₀ of 0.85 μM, which is lower than the 1.21 μM average GI₅₀ for SAHA. Surprisingly, HY-2, HY-3, and HY-5 showed attenuated effects when compared to SAHA with an average GI₅₀ of 2.18, 3.39, and 2.47 μM, respectively. Consistent with further cytotoxicity evaluation and inhibition of purified HDACs results, HC-1 demonstrated lower activity than SAHA with an average GI₅₀ 1.51 μM, demonstrating the contribution to cytotoxicity by ferroptosis. FIG. 21 summarizes the dose response for the selected compounds. All leukemia cell lines showed greater sensitivity to HY-1 over SAHA and corresponding HDAC controls. Although in the one dose assay, the CCRF-CEM cell line showed the highest difference in sensitivity to HY-1 and SAHA, in the five-dose assay RPMI-8226 demonstrated the highest difference due to combined effect of ferroptosis and HDAC inhibition (HY-1 GI₅₀ 0.35 μM, SAHA GI₅₀ 1.5 μM). Consistent with the results of the one dose assay at 10 μM, the NSC lung cancer cell line NCI-H23 showed enhanced sensitivity to HY-1 with GI₅₀ 0.78 μM over SAHA with GI₅₀ 2 μM. The insensitivity of the colon cancer cell lines to ferroptosis was once again confirmed by the similar GI₅₀ values obtained for HY-1 and SAHA against most of the colon cancer cell lines. Contrary to the results of the one dose data of the CNS cancer cell lines, only the SF-539 cell line showed a significant difference in sensitivity to HY-1 and SAHA (HY-1 GI₅₀ 1 μM over SAHA GI₅₀ 2 μM). Of the melanoma cell lines, LOXIMVI showed remarkable sensitivity to the hybrid molecules. HY-1 demonstrated GI₅₀ of 0.02 μM, which is significantly lower than GI₅₀ value of 1.25 μM for SAHA. Of the ovarian cancer cell lines, once again, IGROV-1 demonstrated enhanced sensitivity to hybrid molecules (GI₅₀ 0.57 μM for HY-1 compared to GI₅₀ 2 μM for SAHA). Although the differences were slightly lower when compared to the results of the one dose assay, the renal cancer cell lines (e.g., 786-0 cell line) were more sensitive to the hybrid molecules. Of the two prostate cancer cell lines, PC-3 was slightly more sensitive to SAHA (1.58 μM) compared to HY-1 (GI₅₀ 2 μM), while DU-145 demonstrated opposite effects with GI₅₀ 0.36 μM for HY-1 and GI₅₀ 1.25 μM for SAHA. The breast cancer cell line HS578 showed enhanced sensitivity to HY-1 with GI₅₀ 0.39 μM over 2 μM for SAHA. Analysis of the data in the one dose and five dose assays identifies two basic types of cell lines; those that are significantly more sensitive to hybrid molecules than the HDAC controls and those that have similar effects. Without wishing to be bound by theory, it is believed that the enhanced cytotoxicity results from the combination of ferroptosis with HDAC inhibition.

### Conclusions

In these examples, dual mechanism hybrid molecules capable of inducing ferroptosis and inhibiting HDAC proteins simultaneously are demonstrated. The HDAC inhibitory component of the hybrid molecules was based on a SAHA-like model in which hydroxamates were used as the ZBG, while a short aliphatic chain constituted the linker. A 2-alkynyl thiazole moiety, which is the warhead of CETZOLE ferroptosis inducers, was used as the cap group. The design of the first-generation hybrid molecules focused mainly on the length and the nature of the linker. Hybrid molecules that lacked either the hydroxamate ZBG or ferroptosis-inducing thiazole moiety or both were used as appropriate controls. A SAR study was performed focusing on the length of the linker as well as the unsaturation levels. The results proved the double bond to be a poor bioisosteric change for the amide in the case of the HDAC inhibitors because it provided analogs with attenuated cytotoxicity. In silico studies indicated that the hybrid molecules are capable of binding to HDAC proteins in a similar binding motif as SAHA, with the ZBG occupying the active site and chelating with the zinc ion, and the cap group occupying the outer rim of the proteins. The cytotoxicity of the library of analogues was initially determined on two cell lines; the ferroptosis-sensitive NCI-H522 and the ferroptosis “resistant” HCT-116 cell lines. This combination allowed the delineation of the HDAC inhibitory and ferroptosis-inducing potential of the molecules. Most of the hybrid molecules showed enhanced cytotoxicity on the NCI-H522 cell line when compared to SAHA, CETZOLE-1, or the corresponding HDAC and ferroptosis controls, indicating synergism by the combination of the two mechanisms. In addition, hybrid molecules showed attenuated neurotoxicity. At concentrations capable of showing cytotoxic effects on the NCI-H522 cell line, hybrid molecules showed minimal effect on the neuronal cell line SH-SY5Y, in contrast to the enhanced neuronal toxicity of the ferroptosis inducers and the moderate effects of HDAC controls. These data indicate the ability to use these hybrid molecules as anticancer agents capable of inducing ferroptosis with minimized neuronal side effects. HY-1 demonstrated the best cytotoxicity profile with low nM GI₅₀ values on many cancer cell lines. The first-generation hybrid molecules incorporated a pan-HDAC inhibitor component which demonstrated no isoform selectivity.

### Materials and Methods

All chemicals and solvents were purchased from commercial sources and used without further purification, unless stated otherwise. Anhydrous tetrahydrofuran was freshly distilled from sodium and benzophenone before use. ¹H and ¹³C NMR spectra were recorded on Brucker Avance 600 MHz, INOVA 600 MHz, and Varian VXRS 400 MHz NMR spectrometers in deuterated solvents using residual undeuterated solvents as internal standard. High-resolution mass spectra (HRMS) were recorded on a Waters Synapt high definition mass spectrometer (HDMS) equipped with nano-ESI source. Melting points were determined using a Fisher-Johns melting point apparatus. Purification of crude products was performed by either flash chromatography on silica gel (40-63 μ) from Sorbent Technologies or on a Teledyne ISCO CombiFlash Companion chromatography system on RediSep prepacked silica cartridges. Thin layer chromatography (TLC) plates (20 cm×20 cm) were purchased from Sorbent Technologies (catalog #4115126) and were viewed under Model UVG-54 mineral light lamp UV-254 nm. A Shimadzu Prominence HPLC with an LCT2OAT solvent delivery system coupled to a Shimadzu Prominence SPD 20AV Dual wavelength UV/Vis absorbance Detector, a Shimadzu C₁₈ column (1.9 m, 2.1 mm×50 m) and HPLC grade solvents (MeOH, H₂O with 0.1% formic acid) were used to determine the purity of compounds by HPLC. All compounds were >95% pure by HPLC analysis.

### Synthesis

### General Method for Sonogoshira Coupling

A mixture of the corresponding thiazole-bromide, TMS-acetylene (1.5 equiv.), PdCl₂ (PPh₃)₂ (5 mol %), CuI (6 mol %), Et₃N (2 equiv.), and DCE (5 mL/mmol) was heated under reflux at 83 ° C. for 1 h. The reaction was quenched by the addition of brine, followed by DCM. The organic layer was dried over anhydrous Na₂SO₄, filtered, and concentrated under reduced pressure. The product was purified by flash chromatography on silica in EtOAC/hexanes (0->100% EtOAc) to yield the pure product.

### General Method for Ester Hydrolysis

To a solution of the corresponding ester in freshly distilled THF (2 mL/mmol) and H₂O (1 mL/mmol) was added LiOH (3 equiv.). The resulting mixture was stirred for 2 h at r.t. Most of the THF was removed under reduced pressure, and the remaining aqueous solution was washed with EtOAc, acidified with conc. HCl, and extracted with EtOAc. The organic extract was dried over Na₂SO₄, filtered, and concentrated under reduced pressure to yield the pure product.

### General Method for NH₂ OTHP Coupling

To a stirred suspension of the corresponding carboxylic acid in anhydrous DCM (3 mL/mmol) was added EDC-HCl (1.5 equiv.), followed by the addition of THP-O-NH₂ (1.1 equiv.). The resulting mixture was stirred for 8 h at r.t. The reaction mixture was diluted with brine and extracted with EtOAc. The organic layer was dried over anhydrous Na₂SO₄, filtered, and concentrated under reduced pressure. The crude product was purified by flash chromatography on silica in EtOAC/DCM (0->100% EtOAc) to yield the pure compound.

### General Method for THP Deprotection

To a stirred solution of corresponding THP protected hydroxamic acid in MeOH (3 mL/mmol) was added 1 M HCl (4 equiv.), and the resulting mixture was stirred at r.t. for 3 h. After removal of most of the MeOH under reduced pressure, the residue was diluted with brine and extracted with EtOAC. The organic layer was dried over anhydrous Na₂SO₄, filtered, and concentrated under reduced pressure. The crude product was purified by flash chromatography on silica in DCM/MeOH (0->7% MeOH) to yield pure compound.

### 2-Bromothiazole-5-carboxylic acid (5)

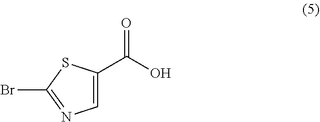

Synthesized according to general hydrolysis conditions. (10 mmol scale, 96% yield). ¹H NMR (600 MHz, DMSO-d₆) δ 13.33 (br, 1H), 8.47 (s, 1H). ¹³C NMR (151 MHz, DMSO-d₆) δ 161.35, 147.58, 137.10, 133.57.

### Methyl 7-aminoheptanoate Hydrochloride salt (7)

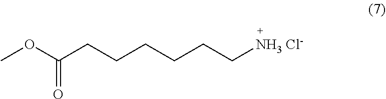

To a stirred solution of 7-aminoheptanoic acid (1.45 g, 10 mmol) in MeOH (30 mL) at 0° C. was added SOCl₂ (7 mL). The resulting mixture was allowed to warm to r.t and stirred at that temperature for 3 hr. The volatiles were removed under reduced pressure to yield the pure product (30) (1.96 g, 100%) as a white solid which was directly used in the next step without any purification. ¹H NMR (600 MHz, DMSO-d₆) δ 8.11 (br, 3H), 3.58 (s, 3H), 2.72 (t, J=7.2 Hz, 2H), 2.30 (t, J=7.4 Hz, 2H), 1.59 -1.46 (m, 4H), 1.34-1.19 (m, 4H). ¹³C NMR (151 MHz, DMSO-d₆) δ 173.80, 51.67, 39.06, 33.57, 28.36, 27.18, 25.97, 24.65.

### Methyl 7-(2-bromothiazole-4-carboxamido)heptanoate (NC-1)

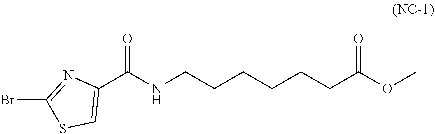 [SMILES: COC(=O)CCCCCCNC(=O)c1csc(Br)n1]

To a stirred suspension of 2-bromothiazole-5-carboxylic acid (5) (416 mg, 2 mmol) in anhydrous DCM (10 mL) were added EDC-HCl (575 mg, 3 mmol, 1.5 equiv.) and DMAP (10 mol %), followed by methyl 7-aminoheptanoate hydrochloric salt (7) (391 mg, 2 mmol, 1 equiv.) and Et₃ N (0.28 mL, 2 mmol, 1 equiv.). The resulting mixture was stirred overnight at r.t, after which it was diluted with brine and DCM. The two layers were separated, and the organic layer was dried over anhydrous Na₂SO₄, filtered, and concentrated under reduced pressure. The crude product was purified by flash chromatography on silica in EtOAC/hexanes (0->100% EtOAc) to yield the pure product (NC-1) (435 mg, 1.24 mmol, 62%) as a colorless oil. ¹H NMR (600 MHz, CDCl₃) δ 8.05 (s, 1H), 7.22 (br, 1H), 3.67 (s, 3H), 3.42 (dt, J=7.2, 3.6 Hz, 2H), 2.31 (t, J=7.5 Hz, 2H), 1.68-1.55 (m, 4H), 1.42-1.33 (m, 4H). ¹³C NMR (151 MHz, CDCl₃) δ 174.16, 159.59, 150.11, 135.72, 126.62, 51.47, 39.37, 33.98, 29.43, 28.75, 26.61, 24.87, 28.75, 26.61 , 24.87. HRMS calcd for C₁₂H₁₇BrN₂ NaO₃ S (M+Na) 371.0040; found 371.0041.

### Methyl 7-(2-ethynylthiazole-4-carboxamido)heptanoate (FC-1)

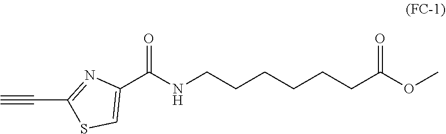 [SMILES: C#Cc1nc(C(=O)NCCCCCCC(=O)OC)cs1]

Synthesized according to general Sonogashira coupling conditions (1 mmol scale, 65% yield). ¹H NMR (600 MHz, CDCl₃) δ 8.13 (s, 1H), 7.33 (br, 1H), 3.67 (s, 3H), 3.56 (s, 1H), 3.44 (dd, J=13.4, 7.0 Hz, 2H), 2.31 (t, J=7.5 Hz, 2H), 1.68-1.58 (m, 4H), 1.45-1.32 (m, 4H). ¹³C NMR (151 MHz, CDCl₃) δ 174.16, 160.17, 150.73, 147.18, 124.93, 83.06, 75.72, 51.50, 39.34, 33.97, 29.43, 28.78, 26.58, 24.79. HRMS calcd for C₁₄H₁₈N₂NaO₃S (M+Na) 317.0935; found 317.0934.

### 7-(2-Bromothiazole-4-carboxamido)heptanoic acid (8)

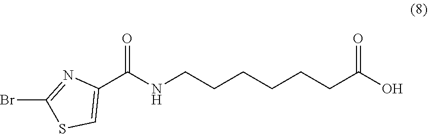 [SMILES: O=C(O)CCCCCCNC(=O)c1csc(Br)n1]

Synthesized according to general hydrolysis conditions. (0.27 mmol scale, 93% yield). ¹H NMR (600 MHz, DMSO-d₆) δ 11.98 (br, 1H), 8.51 (t, J=5.7 Hz, (NH), 1H), 8.25 (s, 1H), 3.21 (dd, J=13.7, 6.6 Hz, 2H), 2.19 (t, J=7.4 Hz, 2H), 1.53-1.44 (m, 4H), 1.30-1.24 (m, 4H). ¹³C NMR (151 MHz, DMSO-d₆) δ 174.96, 159.57, 150.40, 136.64, 128.74, 39.19, 34.08, 29.43, 28.74, 26.61, 24.91.

### 2-Bromo-N-(7-oxo-7-(((tetrahydro-2H-pyran-2-yl)oxy)amino)heptyl)thiazole-4-carboxamide (9)

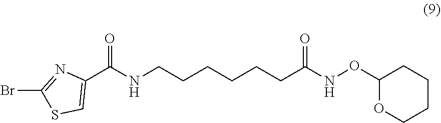 [SMILES: O=C(CCCCCCNC(=O)c1csc(Br)n1)NOC1CCCCO1]

Synthesized according to the general NH₂ OTHP coupling conditions (1.85 mmol scale, 85% yield). ¹H NMR (600 MHz, acetone-d₆) δ 10.22 (br, 1H), 8.23 (s, 1H), 7.96 (br, 1H), 4.93 (s, 1H), 3.97 (t, J=10.6 Hz, 1H), 3.42 (dd, J=13.6, 6.7 Hz, 2H), 2.10 (t, J=7.1 Hz, 2H), 1.78-1.47 (m, 10H), 1.38 (d, J=3.0 Hz, 4H). ¹³C NMR (151 MHz, acetone-d₆) δ 169.54, 159.29, 150.58, 135.59, 127.42, 101.26, 61.40, 38.99, 32.56, 29.45, 28.59, 27.98, 26.44, 25.25, 25.06, 18.42.

### 2-Bromo-N-(7-(hydroxyamino)-7-oxoheptyl)thiazole-4-carboxamide (HC-1)

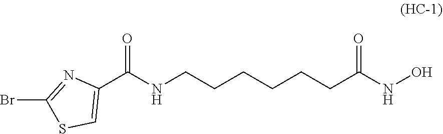 [SMILES: O=C(CCCCCCNC(=O)c1csc(Br)n1)NO]

Synthesized according to the general THP deprotection conditions (0.23 mmol scale, 68% yield). ¹H NMR (600 MHz, DMSO-d₆) δ 10.34 (s, 1H), 8.67 (s, 1H), 8.51 (t, J=5.8 Hz, 1H), 8.25 (s, 1H), 3.21 (dd, J=13.6, 6.7 Hz, 2H), 1.93 (t, J=7.4 Hz, 2H), 1.52-1.44 (m, 4H), 1.30-1.18 (m, 4H). ¹³C NMR (151 MHz, DMSO-d₆) δ 169.55, 159.58, 150.39, 136.66, 128.75, 39.07, 32.69, 29.48, 28.79, 26.61, 25.56. HRMS (ESI) cald for C₁₁H₁₆BrN₃NaO₃S (M+Na) 371.9993; found 371.9991.

### 2-Ethynyl-N-(7-oxo-7-0(tetrahydro-2H-pyran-2-yl)oxy)amino)heptyl)thiazole-4-carboxamide (10)

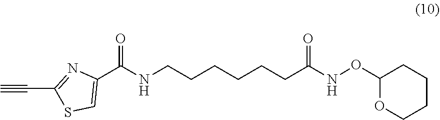 [SMILES: C#Cc1nc(C(=O)NCCCCCCC(=O)NOC2CCCCO2)cs1]

Synthesized according to general Sonogashira coupling conditions. (0.5 mmol scale, 60% yield). ¹H NMR (600 MHz, acetone-d₆) δ 10.10 (br, 1H), 8.27 (s, 1H), 7.94 (br, 1H), 4.92 (s, 1H), 4.43 (s, 1H), 3.96 (t, J=10.6 Hz, 1H), 3.51 (d, J=11.0 Hz, 1H), 3.43 (dd, J=13.6, 6.7 Hz, 2H), 2.13-2.04 (m, J=8.9, 6.3, 4.4 Hz, 3H), 1.79-1.46 (m, 10H), 1.44-1.34 (m, J=3.1 Hz, 4H). ¹³C NMR (151 MHz, acetone-d₆) δ 169.40, 159.70, 151.36, 147.11, 125.20, 101.24, 84.25, 75.68, 61.38, 38.88, 32.51, 29.44, 27.97, 26.40, 25.21, 25.04, 18.41.

### 2-Ethynyl-N-(7-(hydroxyamino)-7-oxoheptyl)thiazole-4-carboxamide (HY-1)

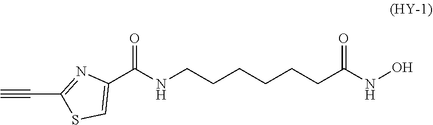 [SMILES: C#Cc1nc(C(=O)NCCCCCCC(=O)NO)cs1]

Synthesized according to the general THP deprotection conditions (0.26 mmol scale, 61% yield). ¹H NMR (600 MHz, acetone-d₆) δ 10.01 (br, 1H), 8.27 (s, 1H), 8.16 (br, J=28.9, 17.3 Hz, 1H), 7.94 (br, 1H), 4.43 (s, 1H), 3.42 (dd, J=13.6, 6.8 Hz, 2H), 2.11 (dd, J=10.3, 4.4 Hz, 2H), 1.67-1.58 (m, 4H), 1.44-1.33 (m, 4H). ¹³C NMR (151 MHz, acetone-d₆) δ 169.81, 159.69, 151.34, 147.11, 125.20, 84.20, 75.71, 38.87, 32.23, 29.41, 26.39, 25.13. HRMS (ESI) calculated for C₁₃H₁₇N₃NaO₃S (M+Na) 318.0888, found 318.0889.

### Ethyl (Z)-8-(2-bromothiazol-4-yl)oct-7-enoate (NC-2)

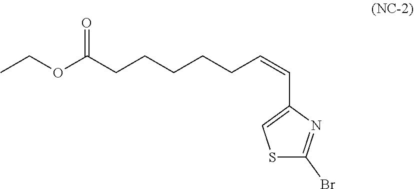 [SMILES: CCOC(=O)CCCCC/C=C\c1csc(Br)n1]

To a round bottom flask containing ethyl 7-bromoheptanoate (S23) (6 g, 35.3 mmol) and triphenyl phosphine (6.6 gr, 25.3 mmol, 1 equiv.) was added toluene (55 mL). The resulting mixture was refluxed for 24 h. Toluene was evaporated under reduced pressure and the residue was washed with hexanes and diethyl ether and dried under vacuum to obtain the phosphonium bromide salt (S24), which was used in the next step without further purification.

To a stirred solution of 2-bromothiazole-4-carbaldehyde (2) (prepared as shown in Scheme 1, FIG. 4) (2.5 g, 13 mmol) and (7-ethoxy-7-oxoheptyl)triphenylphosphonium bromide (S24) (6.6 g, 13 mmol, 1 equiv.) in DCM (20 mL) was added dropwise a solution of NaOH (8 mL of 50% solution in H₂O). The resulting biphasic mixture was stirred at r.t for 2 h and partitioned between DCM and brine. The organic layer was dried over anhydrous Na₂SO₄, filtered, and concentrated under reduced pressure. The crude product mixture was separated on silica in EtOAc/Hex (0->20% EtOAc), providing some fractions with pure Z-isomer (NC-2) (1.9 g, 5.85 mmol, 45%). ¹H NMR (600 MHz, CDCl₃) δ 7.03 (s, 1H), 6.38 (dt, J=11.7, 1.6 Hz, 1H), 5.77 (dt, J=11.7, 7.3 Hz, 1H), 4.15 (q, J=7.1 Hz, 2H), 2.55 (qd, J=7.4, 1.7 Hz, 2H), 2.34-2.30 (m, 2H), 1.68 (dt, J=15.2, 7.5 Hz, 2H), 1.51 (dd, J=15.1, 7.6 Hz, 2H), 1.44-1.38 (m, 2H), 1.27 (t, J=7.1 Hz, 3H). ¹³ C NMR (151 MHz, CDCl₃) δ 173.83, 153.87, 135.56, 134.72, 121.16, 119.34, 60.22, 34.30, 29.13, 28.79, 24.83, 14.28.

Some fractions with E/Z mixture (1.3 g, 3.9 mmol, 30%) were obtained as well and were used to obtain a mixture of E/Z analogs.

### Ethyl (Z)-8-(2-ethynylthiazol-4-yl)oct-7-enoate (FC-2)

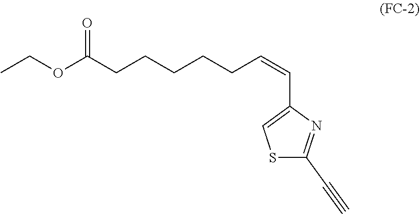 [SMILES: C#Cc1nc(/C=C\CCCCCC(=O)OCC)cs1]

Synthesized according to general Sonogashira coupling conditions (2 mmol scale, 62.5% yield). ¹H NMR (600 MHz, CDCl₃) δ 7.12 (s, 1H), 6.45 (dt, J=11.7, 1.7 Hz, 1H), 5.81 (dt, J=11.7, 7.2 Hz, 1H), 4.14 (q, J=7.1 Hz, 2H), 2.57 (qd, J=7.4, 1.7 Hz, 2H), 2.32 (t, J=7.5 Hz, 2H), 1.67 (dt, J=15.2, 7.6 Hz, 2H), 1.52 (dd, J=15.1, 7.6 Hz, 2H), 1.45-1.38 (m, 2H), 1.27 (t, J=7.1 Hz, 3H). ¹³C NMR (151 MHz, CDCl₃) δ 173.84, 154.20, 146.21, 135.70, 121.49, 117.89, 81.82, 76.68, 60.22, 34.31, 29.15, 28.95, 28.80, 24.85, 14.28.

### (Z)-8-(2-bromothiazol-4-yl)-N-hydroxyoct-7-enamide (HC-2)

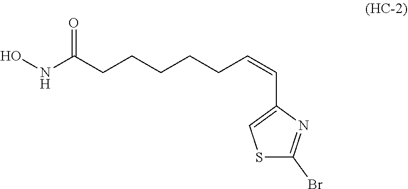 [SMILES: O=C(CCCCC/C=C\c1csc(Br)n1)NO]

Synthesized according to the general THP deprotection conditions (0.1 mmol scale, 76% yield). ¹H NMR (600 MHz, acetone-d₆) δ 9.94 (s, 1H), 7.86 (s, 1H), 7.46 (s, 1H), 6.38 (d, J=11.7 Hz, 2H), 5.80-5.74 (m, 1H), 2.61 (q, J=7.4 Hz, 2H), 2.11 (t, J=7.3 Hz, 2H), 1.64 (dt, J=14.9, 7.5 Hz, 2H), 1.50 (dt, J=14.9, 7.3 Hz, 2H), 1.39 (dt, J=15.2, 7.6 Hz, 2H). ¹³ C NMR (151 MHz, acetone-d₆) δ 169.59, 153.97, 134.93, 134.30, 122.34, 120.92, 32.26, 29.49, 29.04, 28.55, 25.16. HRMS (ESI) calcd for C₁₁H₁₆BrN₂O₂S (M+H) 319.0115 found 319.0122.

### (Z)-8-(2-ethynylthiazol-4-yl)-N-hydroxyoct-7-enamide (HY-2)

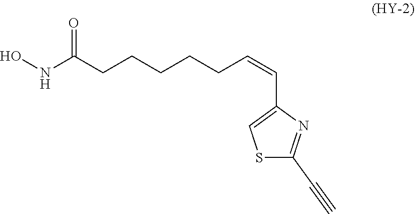 [SMILES: C#Cc1nc(/C=C\CCCCCC(=O)NO)cs1]

Synthesized according to the general THP deprotection conditions (0.1 mmol scale, 75% yield). ¹H NMR (600 MHz, acetone-d₆) δ 9.96 (s, 1H), 8.01 (s, 1H), 7.53 (s, 1H), 6.44 (d, J=11.7 Hz, 1H), 5.80 (dt, J=11.7, 7.3 Hz, 1H), 4.30 (s, 1H), 2.64 (dd, J=13.8, 7.1 Hz, 2H), 2.11 (t, J=7.3 Hz, 2H), 1.67-1.61 (m, 2H), 1.50 (dt, J=15.1, 7.5 Hz, 2H), 1.38 (dd, J=14.9, 7.0 Hz, 2H). ¹³C NMR (151 MHz, acetone-d₆) δ 169.69, 154.36, 146.10, 135.08, 121.22, 119.13, 83.04, 76.56, 32.26, 29.05, 28.84, 28.72, 25.20. HRMS (ESI) calcd for C₁₃H₁₇N₂O₂S (M+H) 265.1010, found 265.1017.

### Methyl (2E,4E)-5-(2-bromothiazol-4-yl)penta-2,4-dienoate (NC-4)

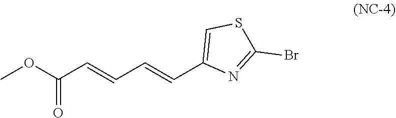

To a round bottom flask containing methyl (E)-4-bromobut-2-enoate (S28) (3.8 g, 21.7 mmol, 1 equiv.) was added triethyl phosphite (3.77 mL, 21.7 mmol, 1 equiv). The resulting mixture was heated at 160° C. for 30 min, after which point it was allowed to cool to room temperature. The prepared methyl (E)-4-(diethoxyphosphoryl)but-2-enoate (S29) was added to a stirred solution of 2-bromothiazole-4-carbaldehyde (4.1 gr, 21.7 mmol) in DME (50 mL), followed by the dropwise addition of freshly prepared 1 M MeONa in MeOH (21.7 mL, 1 equiv.). The resulting mixture was stirred at r.t. for 30 min, transferred to a flask containing iced water, and stirred for an additional 15 min. EtOAc was added and the organic layer was collected, dried over anhydrous Na₂SO₄, filtered, and concentrated under reduced pressure. The crude product mixture was purified by silica gel chromatography in EtOAc/hexanes (0->100% EtOAc) to yield pure compound (NC-4) (3.7 g, 13.4 mmol, 62%). ¹H NMR (600 MHz, CDCl₃) δ 7.42 (ddd, J=15.2, 11.5, 0.5 Hz, 1H), 7.24-7.19 (m, 1H), 7.19 (s, 1H), 6.78 (d, J=9.3 Hz, 1H), 6.07 (d, J=15.3 Hz, 1H), 3.79 (s, 3H). ¹³C NMR (151 MHz, CDCl₃) δ 167.18, 153.10, 143.70, 136.79, 130.48, 129.82, 122.92, 120.84, 51.76.

### Methyl (2E,4E)-5-(2-ethynylthiazol-4-yl)penta-2,4-dienoate (FC-4)

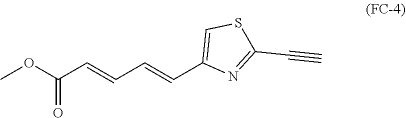

Synthesized according to general Sonogashira coupling conditions (0.66 mmol scale, 60% yield). ¹H NMR (600 MHz, CDCl₃) δ 7.49-7.40 (m, 1H), 7.33-7.25 (m, 2H), 6.85 (d, J=15.2 Hz, 1H), 6.08 (d, J=15.2 Hz, 1H), 3.80 (s, 3H), 3.53 (s, 1H). ¹³C NMR (151 MHz, CDCl₃) δ 167.37, 153.60, 147.90, 143.86, 130.93, 130.09, 122.70, 119.76, 82.82, 76.22, 51.68. HRMS (ESI) calcd for C₁₁H₁₀NO₂S (M+H) 220.0432, found 220.0441.

### (2E,4E)-5-(2-bromothiazol-4-yl)penta-2,4-dienoic acid (16)

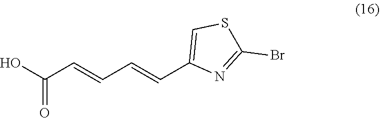 [SMILES: O=C(O)/C=C/C=C/c1csc(Br)n1]

Synthesized according to general hydrolysis conditions. (0.11 mmol scale, 88% yield). 1 H NMR (600 MHz, DMSO d₆) δ 12.37 (s, 1H), 7.81 (s, 1H), 7.32 (dd, J=15.1, 11.3 Hz, 1H), 7.14 (dd, J=15.1, 11.3 Hz, 1H), 7.03 (d, J=15.1 Hz, 1H), 6.11 (d, J=15.2 Hz, 1H). ¹³C NMR (151 MHz, DMSO d₆) δ 167.91, 153.21, 143.86, 137.53, 131.41, 129.53, 124.46, 124.11.

### (2E,4E)-5-(2-bromothiazol-4-yl)-N-((tetrahydro-2H-pyran-2-yl)oxy)penta-2,4-dienamide (17)

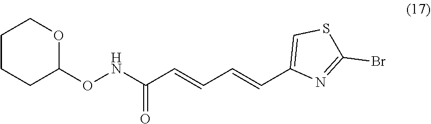 [SMILES: O=C(/C=C/C=C/c1csc(Br)n1)NOC1CCCCO1]

Synthesized according to the general NH₂ OTHP coupling conditions (0.1 mmol scale, 93% yield). ¹H NMR (600 MHz, acetone d₆) δ 10.38 (s, 1H), 7.66 (s, 1H), 7.40-7.34 (m, 1H), 7.18 (t, J=12.3 Hz, 1H), 6.97 (d, J=15.0 Hz, 1H), 6.20 (d, J=15.0 Hz, 1H), 4.99 (s, 1H), 4.00 (t, J=10.5 Hz, 1H), 3.54 (d, J=10.9 Hz, 1H), 1.88-1.65 (m, 3H), 1.66-1.48 (m, 3H). ¹³C NMR (151 MHz, acetone d₆) δ 153.70, 139.47, 136.21, 129.75, 123.20, 122.25, 101.49, 61.51, 27.98, 25.01, 18.42.

### (2E,4E)-5-(2-bromothiazol-4-yl)-N-hydroxypenta-2,4-dienamide (HC-4)

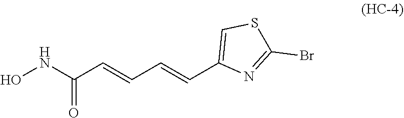 [SMILES: O=C(/C=C/C=C/c1csc(Br)n1)NO]

Synthesized according to the general THP deprotection conditions (0.07 mmol scale, 50% yield). ¹H NMR (600 MHz, MeOD-d₄) δ 7.54 (s,1H), 7.36-7.30 (m, J=14.5 Hz, 1H), 7.19-7.13 (m, J=14.8, 11.5 Hz, 1H), 6.92-6.85 (m, J=15.1 Hz, 1H), 6.09-6.03 (m, J=12.8 Hz, 1H). ¹³C NMR (151 MHz, MeOD d₄) δ 164.86, 153.52, 139.46, 136.77, 129.58, 129.40, 121.84. HRMS (ESI) calcd for C₈H₈BrN₂O₂S (M+H) 274.9489, found 274.9479.

### (2E,4E)-5-(2-ethynylthiazol-4-yl)-N-((tetrahydro-2H-pyran-2-yl)oxy)penta-2,4-dienamide (18)

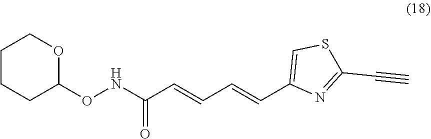 [SMILES: C#Cc1nc(/C=C/C=C/C(=O)NOC2CCCCO2)cs1]

Synthesized according to general Sonogashira coupling conditions (0.35 mmol scale, 70% yield). ¹H NMR (600 MHz, CDCl₃) δ 8.82 (br, 1H), 7.55-7.42 (m, 1H), 7.30-7.27 (m, 1H), 6.83 (d, J=15.1 Hz, 1H), 5.94 (br, J=69.6 Hz, 1H), 5.01 (s, 1H), 4.05-3.94 (m, 1H), 3.79-3.74 (m, 1H), 3.66 (ddd, J=11.1, 5.5, 4.1 Hz, 1H), 1.87-1.82 (m, 3H), 1.67-1.61 (m, 3H). ¹³C NMR (151 MHz, CDCl₃) δ 207.71, 157.04, 153.28, 152.99, 147.83, 130.54, 130.15, 127.52, 121.34, 119.54, 100.81, 82.50, 76.24, 62.66, 28.07, 24.69, 18.66.

### (2E,4E)-5-(2-ethynylthiazol-4-yl)-N-hydroxypenta-2,4-dienamide (HY-4)

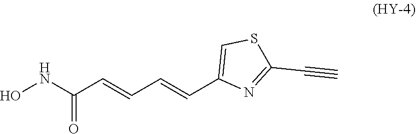

Synthesized according to the general THP deprotection conditions (0.063 mmol scale, 63% yield) using TFA/DCM instead of HCl/MeOH. ¹H NMR (600 MHz, acetone d₆) δ 10.36 (s, 1H), 8.11 (s, 1H), 7.73 (s, 1H), 7.35 (t, J=13.3 Hz, 1H), 7.27-7.21 (m, 1H), 7.00 (d, J=14.9 Hz, 1H), 6.18 (d, J=14.7 Hz, 1H), 4.37 (s, 1H). ¹³C NMR (151 MHz, acetone d₆) δ 163.23, 153.99, 147.41, 138.92, 130.03, 129.88, 122.58, 120.15, 83.64, 76.23. HRMS (ESI) calcd for C₁₀H₉N₂O₂S (M+H) 221.0384, found 221.0962.

### Methyl (E)-3-(2-bromothiazol-4-yl)acrylate (NC-5)

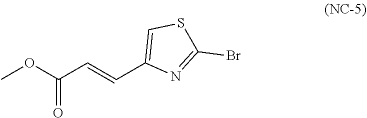

To a stirred solution of triphenyl phosphine (2.3 g, 9 mmol, 1.05 equiv.) in EtOAc (14 mL) was slowly added a solution of bromomethyl acetate (S30) (1.3 g, 8.5 mmol) in EtOAc (2.5 mL). The resulting mixture was stirred at r.t for 12 h. The precipitated white solid was collected by filtration, washed with diethyl ether, and air-dried overnight. It was resuspended in 1 N NaOH (16 mL) and stirred for 20 min, at which point DCM was added. The organic layer was washed with brine, dried over anhydrous Na₂SO₄, filtered, and concentrated under vacuum to provide ylide (19), which was used in the next step without further purification.

The ylide (19) synthesized as described above (9 mmol) and 2-bromothiazole-4-carbaldehyde (2) (1.7 g, 9 mmol, 1 equiv) were dissolved in THF (52 mL) and stirred at r.t for 12 h. Brine and DCM were added and the organic layer was separated and dried over anhydrous Na₂SO₄, filtered, and concentrated under vacuum. The crude product was purified by silica gel chromatography in EtOAc/Hexanes (0->100% EtOAc) to provide the pure product (NC-5) (2 g, 8.1 mmol, 90%). ¹H NMR (600 MHz, CDCl₃) δ 7.55-7.49 (m, 1H), 7.37 (s, 1H), 6.77 (d, J=15.5 Hz, 1H), 3.80 (s, 3H). ¹³C NMR (151 MHz, CDCl₃) δ 167.19, 151.69, 137.37, 134.50, 124.78, 121.49, 52.42. HRMS (ESI) calcd for C₇H₇BrNO₂S (M+H) 247.9380, found 247.9382.

### Methyl (E)-3-(2-ethynylthiazol-4-yl)acrylate (FC-5)

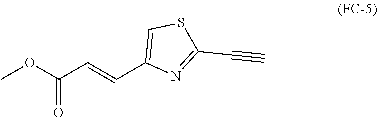

Synthesized according to general Sonogashira coupling conditions (0.415 mmol scale, 83% yield). ¹H NMR (600 MHz, CDCl₃) δ 7.60 (d, J=15.6 Hz, 1H), 7.46 (s, 1H), 6.84 (d, J=15.6 Hz, 1H), 3.82 (s, 3H), 3.54 (s, 1H). ¹³C NMR (151 MHz, CDCl₃) δ 167.34, 152.04, 148.16, 135.40, 122.77, 121.61, 82.79, 75.98, 51.77. HRMS (ESI) calcd for C₉H₈NO₂S (M+H) 194.0275, found 194.0279.

### (E)-3-(2-bromothiazol-4-yl)acrylic acid (20)

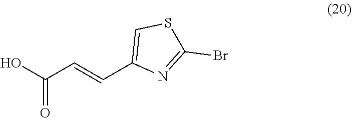 [SMILES: O=C(O)/C=C/c1csc(Br)n1]

Synthesized according to general hydrolysis conditions. (2.17 mmol scale, 99% yield). ¹H NMR (600 MHz, DMSO-d₆) δ 12.54 (s, 1H), 8.10 (s, 1H), 7.53 (d, J=15.5 Hz, 1H), 6.48 (d, J=15.5 Hz, 1H). 13C NMR (151 MHz, DMSO-d₆) δ 167.75, 151.43, 138.01, 135.71, 128.28, 121.84.

### (E)-3-(2-bromothiazol-4-yl)-N-((tetrahydro-2H-pyran-2-yl)oxy)aciylamide (21)

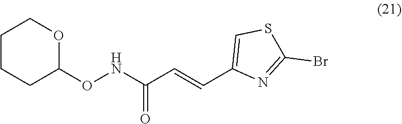 [SMILES: O=C(/C=C/c1csc(Br)n1)NOC1CCCCO1]

Synthesized according to the general NH₂ OTHP coupling conditions (2.11 mmol scale, 88% yield). ¹H NMR (600 MHz, acetone d₆) δ 10.42 (s, 1H), 7.86 (s, 1H), 7.53 (d, J=15.3 Hz, 1H), 6.79 (t, J=13.7 Hz, 1H), 5.02 (s, 1H), 4.02 (t, J=10.5 Hz, 1H), 3.58-3.54 (m, 1H), 1.81-1.74 (m, 3H), 1.63-1.56 (m, 3H). ¹³C NMR (151 MHz, acetone d₆) δ 162.64, 152.48, 136.59, 133.36, 131.35, 101.43, 67.43, 61.48, 27.94, 27.32, 25.04, 25.00, 22.77, 18.35.

### (E)-3-(2-bromothiazol-4-yl)-N-hydroxyacrylamide (HC-5)

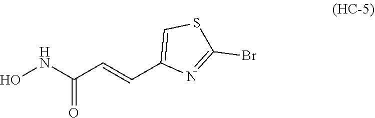 [SMILES: O=C(/C=C/c1csc(Br)n1)NO]

Synthesized according to the general THP deprotection conditions (0.29 mmol scale, 97% yield). ¹H NMR (600 MHz, DMSO-d₆) δ 10.83 (S, 1H), 9.12 (d, J=1.7 Hz, 1H), 7.95 (s, 1H), 7.40 (d, J=15.3 Hz, 1H), 6.63 (d, J=15.3 Hz, 1H). ¹³C NMR (151 MHz, DMSO-d₆) δ 162.61, 151.88, 137.57, 130.78, 126.63, 121.85. HRMS (ESI) calcd for C₆H₆BrN₂O₂S (M+H) 248.9333, found 248.9345.

### (E)-3-(2-ethynylthiazol-4-yl)-N-((tetrahydro-2H-pyran-2-yl)oxy)aciylamide (22)

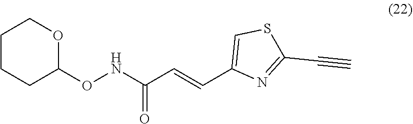 [SMILES: C#Cc1nc(/C=C/C(=O)NOC2CCCCO2)cs1]

Synthesized according to general Sonogashira coupling conditions (1.16 mmol scale, 83% yield). ¹H NMR (600 MHz, acetone d₆) δ 10.45 (s, 1H), 7.96 (s, 1H), 7.61 (d, J=15.8 Hz, 2H), 6.90 (d, J=15.8 Hz, 1H), 5.05 (s, 1H), 4.41 (s, 1H), 4.06 (s, 1H), 3.59 (s, 1H), 1.80 (m, 3H), 1.62 (m, 3H). ¹³C NMR (151 MHz, acetone d₆) δ 162.60, 152.64, 147.77, 131.74, 128.57, 123.06, 121.73, 101.50, 83.89, 76.11, 61.50, 27.99, 25.05, 18.39.

### (E)-3-(2-ethynylthiazol-4-yl)-N-hydroxyacrylamide (HY-5)

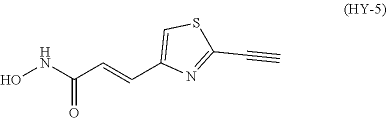

Synthesized according to the general THP deprotection conditions (0.29 mmol scale, 96% yield). ¹H NMR (600 MHz, DMSO-d₆) δ 10.85 (s, 1H), 9.12 (s, 1H), 8.05 (s, 1H), 7.44 (d, J=15.3, 6.9 Hz, 1H), 6.68 (d, J=15.3 Hz, 1H), 5.02 (s, 1H). ¹³C NMR (151 MHz, DMSO-d₆) δ 162.79, 152.45, 147.73, 131.00, 124.14, 122.28, 87.09, 76.58. HRMS (ESI) calcd for C₈H₇N₂O₂S (M+H) 195.0228, found 195.0222.

### Ethyl (E)-8-(2-bromothiazol-4-yl)oct-7-enoate (NC-3)

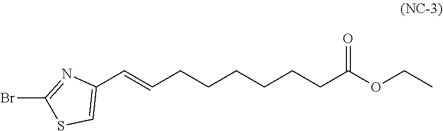 [SMILES: CCOC(=O)CCCCCC/C=C/c1csc(Br)n1]

Synthesized as previously described for the synthesis of compound (14) using LiOH as the base instead of NaOH (1.41 mmol scale, 70% yield). ¹H NMR (600 MHz, CDCl₃) δ 6.88 (s, 1H), 6.63-6.56 (m, 1H), 6.31 (d, J=14.6 Hz, 1H), 4.17-4.11 (m, 2H), 2.35-2.28 (m, 2H), 2.26-2.17 (m, 2H), 1.70-1.62 (m, J=6.9 Hz, 2H), 1.54-1.47 (m, 2H), 1.42-1.35 (m, 2H), 1.30-1.24 (m, 3H). ¹³C NMR (151 MHz, CDCl₃) δ 173.80, 154.76, 135.84, 135.27, 121.98, 116.28, 60.24, 34.29, 32.43, 28.66, 28.60, 24.80, 14.27. HRMS (ESI) calcd for C₁₃H₁₉BrNO₂S (M+H) 334.0319, found 334.0311.

### Ethyl (E)-8-(2-ethynylthiazol-4-yl)oct-7-enoate (FC-3)

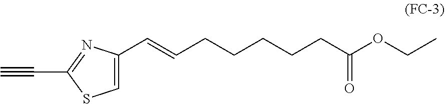 [SMILES: C#Cc1nc(/C=C/CCCCCC(=O)OCC)cs1]

Synthesized according to general Sonogashira coupling conditions (0.268 mmol scale, 67% yield). ¹H NMR (600 MHz, CDCl₃) δ 6.99 (s, 1H), 6.68-6.62 (m, 1H), 6.39 (dd, J=15.6, 1.4 Hz, 1H), 4.17-4.12 (m, 2H), 3.47 (s, 1H), 2.31 (dd, J=9.7, 5.4 Hz, 2H), 2.26-2.22 (m, 2H), 1.66 (dt, J=15.4, 7.6 Hz, 2H), 1.51 (dt, J=15.2, 7.6 Hz, 2H), 1.44-1.36 (m, 3H), 1.27 (t, J=7.1 Hz, 3H). 13 C NMR (151 MHz, CDCl₃) δ 173.82, 155.04, 147.05, 135.40, 122.29, 114.94, 81.92, 76.61, 60.22, 34.30, 32.53, 29.71, 28.67, 28.61, 24.81, 14.26. HRMS (ESI) calcd for C₁₅H₂₀NO₂S (M+H) 278.1214, found 278.1242.

### (E)-8-(2-bromothiazol-4-yl)-N-hydroxyoct-7-enamide (HC-3)

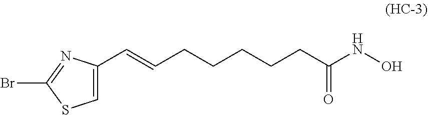 [SMILES: O=C(CCCCC/C=C/c1csc(Br)n1)NO]

Synthesized according to the general THP deprotection conditions (0.04 mmol scale, 77% yield). ¹H NMR (600 MHz, MeOH-d₄) δ 7.22 (s, 1H), 6.58-6.51 (m, 1H), 6.38 (d, J=15.6 Hz, 1H), 3.66 (s, 1H), 2.25 (q, J=6.9 Hz, 2H), 2.12 (t, J=7.4 Hz, 2H), 1.67 (dt, J=15.0, 7.5 Hz, 3H), 1.52 (dd, J=15.0, 7.5 Hz, 2H), 1.41 (dd, J=15.3, 8.3 Hz, 3H), 0.92 (t, J=6.8 Hz, 2H). 13 C NMR (151 MHz, MeOH d₄) δ 171.53, 154.59, 135.81, 134.41, 121.96, 117.06, 32.28, 32.02, 28.40, 28.23, 25.19. HRMS (ESI) calcd for C₁₁H₁₆BrN₂O₂S (M+H) 319.0115, found 319.0122.

### (E)-8-(2-ethynylthiazol-4-yl)-N-hydroxyoct-7-enamide (HY-3)

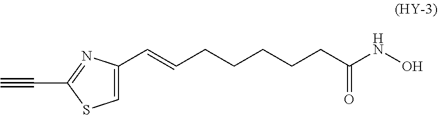 [SMILES: C#Cc1nc(/C=C/CCCCCC(=O)NO)cs1]

Synthesized according to the general THP deprotection conditions (0.105 mmol scale, 75% yield). ¹H NMR (600 MHz, acetone-d₆) δ 9.95 (s, 1H), 7.86 (s, 1H), 7.42 (s, 1H), 6.66-6.61 (m, 2H), 6.48 (d, J=15.6 Hz, 2H), 4.29 (s, 1H), 2.24 (q, J=6.9 Hz, 3H), 2.12 (dd, J=12.0, 4.6 Hz, 3H), 1.64 (dt, J=15.1, 7.5 Hz, 4H), 1.50 (dd, J=15.0, 7.5 Hz, 4H), 1.38 (dt, J=15.0, 7.6 Hz, 4H). ¹³C NMR (151 MHz, acetone-d₆) δ 169.58, 155.07, 146.68, 134.76, 122.64, 115.78, 82.96, 76.54, 32.22, 28.57, 28.49, 25.12. HRMS (ESI) calcd for C₁₃H₁₇N₂O₂S (M+H) 265.1010, found 265.1034.

### 5 -Methoxy-5 -oxopentan-1-aminium chloride (S2)

Synthesized following similar protocol for the synthesis of compound (7). 100% yield. ¹H NMR (600 MHz, DMSO) δ 7.99 (s, 3H), 3.58 (s, 3H), 2.75 (s, 2H), 2.34 (s, 2H), 1.56 (s, 4H). ¹³C NMR (151 MHz, DMSO) δ 173.56, 51.75, 38.80, 33.11, 26.82, 21.82.

### Methyl 5-(2-bromothiazole-4-carboxamido)pentanoate (S3)

Synthesized following similar protocol for the synthesis of (NC-1). 36% yield. ¹H NMR (600 MHz, CDCl₃) δ 8.03 (s, 1H), 7.24 (d, J=5.6 Hz, 1H), 3.67 (s, 3H), 3.44 (dd, J=13.1, 6.8 Hz, 2H), 2.36 (t, J=7.3 Hz, 2H), 1.75-1.60 (m, 4H). ¹³C NMR (151 MHz, CDCl₃) δ 173.81, 159.71, 150.01, 135.82, 126.70, 51.63, 39.01, 33.56, 29.06, 22.21.

### 5-(2-Bromothiazole-4-carboxamido)pentanoic acid (S4)

Synthesized according to general hydrolysis conditions. 92% yield. ¹H NMR (600 MHz, DMSO) δ 8.54 (t, J=6.0 Hz, 1H), 8.20 (s, 1H), 3.26-3.18 (m, 2H), 2.21 (t, J=6.9 Hz, 2H), 1.50-1.45 (m, 4H). ¹³C NMR (151 MHz, DMSO) δ 175.31, 160.00, 149.95, 136.92, 128.84, 38.82, 33.71, 28.88, 22.23.

### 2-Bromo-N-(5-oxo-5-(((tetrahydro-2H-pyran-2-yl)oxy)amino)pentyl)thiazole-4-carboxamide (S5)

Synthesized according to the general NH₂ OTHP coupling conditions. 72% yield. ¹HNMR (600 MHz, CDCl₃) δ 9.19 (s, 1H), 8.07 (s, 1H), 7.38 (s, 1H), 4.97 (s, 1H), 3.97 (t, J=9.9 Hz, 1H), 3.65-3.58 (m, 1H), 3.45 (dd, J=12.8, 6.3 Hz, 2H), 2.23 (dd, J=20.5, 13.0 Hz, 2H), 1.84-1.66 (m, 10H). ¹³C NMR (151 MHz, CDCl₃) δ 170.29, 160.01, 149.80, 135.95, 126.96, 102.36, 94.50, 62.44, 38.67, 32.46, 28.84, 27.99, 25.02, 22.51, 18.53.

### 2-Ethynyl-N-(5-oxo-5-(((tetrahydro-2H-pyran-2-yl)oxy)amino)pentyl)thiazole-4-carboxamide (S6)

Synthesized according to general Sonogashira coupling conditions. 58% yield. ¹H NMR (400 MHz, CDCl₃) δ 8.81 (s, 1H), 8.10 (s, 1H), 7.40 (s, 1H), 4.94 (s, 1H), 3.94 (t, J=9.6 Hz, 1H), 3.65-3.55 (m, 1H), 3.53 (s, 1H), 3.49-3.37 (m, 4H), 1.81-1.58 (m, 10H). ¹³C NMR (151 MHz, CDCl₃) δ 170.24, 160.46, 150.47, 147.24, 125.08, 102.33, 83.16, 75.65, 62.40, 38.60, 32.52, 29.70, 28.87, 27.98, 25.02, 22.56, 18.41.

### 2-Ethynyl-N-(5-(hydroxyamino)-5-oxopentyl)thiazole-4-carboxamide (S7)

Compound S7 was synthesized using the general THP deprotection conditions. 14% yield. ¹H NMR (600 MHz, acetone-d₆) δ 10.04 (s, 1H), 8.27 (s, 1H), 8.23 (s, 1H), 7.95 (s, 1H), 4.43 (s, 1H), 3.47-3.41 (m, 2H), 2.17 (t, J=6.9 Hz, 2H), 1.73-1.60 (m, 4H). ¹³C NMR (151 MHz, acetone-d₆) δ 169.79, 159.80, 151.24, 147.14, 125.30, 84.24, 75.69, 38.49, 31.90, 29.02, 22.62.

### 6-Methoxy-6-oxohexan-l-aminium chloride (S9)

Synthesized following similar protocol for the synthesis of compound (7). 100% yield. ¹H NMR (600 MHz, DMSO) δ 8.11 (s, 3H), 3.58 (s, 3H), 2.76-2.67 (m, 2H), 2.34-2.22 (m, 2H), 1.61-1.45 (m, 4H), 1.34-1.25 (m, 2H). ¹³C NMR (151 MHz, DMSO) δ 173.27, 51.28, 38.49, 33.06, 26.59, 25.33, 23.92.

### Methyl 6-(2-bromothiazole-4-carboxamido)hexanoate (S10)

Synthesized following similar protocol for the synthesis of (NC-1). 53% yield. ¹H NMR (600 MHz, CDCl₃) δ 8.03 (s, 1H), 7.22 (s, J=10.8 Hz, 1H), 3.66 (s, 3H), 3.43 (dd, J=13.6, 6.8 Hz, 2H), 2.32 (t, J=7.5 Hz, 2H), 1.71-1.65 (m, 2H), 1.65-1.59 (m, 2H), 1.44-1.37 (m, 2H). ¹³C NMR (151 MHz, CDCl₃) δ 174.16, 159.79, 150.19, 135.91, 126.80, 51.68, 39.37, 34.02, 29.44, 26.54, 24.68.

### 6-(2-Bromothiazole-4-carboxamido)hexanoic acid (S11)

Synthesized according to general hydrolysis conditions. 92% yield. ¹H NMR (600 MHz, DMSO) δ 11.99 (s, 1H), 8.51 (t, J=5.8 Hz, 1H), 8.25 (s, 1H), 3.21 (dd, J=13.5, 6.7 Hz, 2H), 2.19 (t, J=7.4 Hz, 2H), 1.54-1.45 (m, 4H), 1.30-1.23 (m, 2H). ¹³C NMR (151 MHz, DMSO) δ 174.95, 159.58, 150.38, 136.66, 128.76, 39.08, 34.04, 29.31, 26.39, 24.69.

### 2-Bromo-N-(6-oxo-6-(((tetrahydro-2H-pyran-2-yl)oxy)amino)hexyl)thiazole-4-carboxamide (S12)

Synthesized according to the general NH₂ OTHP coupling conditions. 79% yield. ¹H NMR (600 MHz, CDCl₃) δ 9.20 (s, 1H), 8.04 (s, 1H), 7.31 (s, 1H), 4.93 (s, 1H), 3.93 (t, J=9.8 Hz, 1H), 3.57 (d, J=11.1 Hz, 1H), 3.38 (dd, J=13.2, 6.6 Hz, 2H), 2.10 (s, J=6.5 Hz, 2H), 1.80-1.54 (m, 12H). ¹³C NMR (151 MHz, CDCl₃) δ 170.43, 159.83, 149.93, 135.88, 126.90, 102.35, 62.43, 39.28, 33.04, 29.24, 28.00, 25.01, 18.57.

### 2-Ethynyl-N-(6-oxo-6-0(tetrahydro-2H-pyran-2-yl)oxy)amino)hexyl)thiazole-4-carboxamide (S13)

Synthesized according to general Sonogashira coupling conditions. 55% yield. ¹H NMR (400 MHz, CDCl₃) δ 9.21 (s, 1H), 8.11 (s, 1H), 7.39 (s, 1H), 4.92 (s, 1H), 3.92 (t, J=9.0 Hz, 1H), 3.59-3.52 (m, 2H), 3.38 (dd, J=13.2, 6.6 Hz, 2H), 2.10 (s, 2H), 1.77-1.51 (m, 12H). ¹³C NMR (151 MHz, CDCl₃) δ 170.46, 160.33, 150.49, 147.28, 125.18, 102.20, 83.46, 75.63, 62.27, 39.27, 32.99, 29.23, 27.98, 26.37, 25.00, 18.53.

### 2-Ethynyl-N-(6-(hydroxyamino)-6-oxohexyl)thiazole-4-carboxamide (S14)

Compound S14 was synthesized using the general THP deprotection conditions. 24% yield. ¹H NMR (600 MHz, acetone-d₆) δ 10.02 (s, 1H), 8.27 (s, 1H), 8.27 (s, 1H), 7.95 (s, 1H), 4.43 (s, 1H), 3.42 (dd, J=13.5, 6.7 Hz, 2H), 2.13 (t, J=7.3 Hz, 2H), 1.71-1.59 (m, 4H), 1.45-1.36 (m, 2H). ¹³ C NMR (151 MHz, acetone-d₆) δ 169.75, 159.72, 151.31, 147.12, 125.24, 84.23, 75.70, 38.83, 32.24, 26.24, 25.00.

### 8-Methoxy-8-oxooctan-1-aminium chloride (S16)

Synthesized following similar protocol for the synthesis of compound (7). 100% yield. 1 H NMR (600 MHz, DMSO) δ 7.80 (s, 3H), 3.58 (s, 3H), 2.76-2.72 (m, 2H), 2.31-2.27 (m, 2H), 1.56-1.46 (m, 4H), 1.31-1.21 (m, 6H). ¹³C NMR (151 MHz, DMSO) δ 173.84, 51.67, 39.19, 33.68, 28.70, 28.62, 27.38, 26.09, 24.76.

### Methyl 8-(2-bromothiazole-4-carboxamido)octanoate (S17)

Synthesized following similar protocol for the synthesis of NC-1. 40% yield. ¹¹H NMR (600 MHz, CDCl₃) δ 8.03 (s, J=1.0 Hz, 1H), 7.20 (s, 1H), 3.66 (s, 3H), 3.41 (dd, J=13.5, 6.9 Hz, 2H), 2.30 (t, J=7.5 Hz, 2H), 1.66-1.55 (m, 4H), 1.41-1.27 (m, 6H). ¹³C NMR (151 MHz, CDCl₃) δ 174.28, 159.64, 150.14, 135.76, 126.62, 51.51, 39.47, 34.05, 29.54, 29.01, 28.93, 26.75, 24.86.

### 8-(2-Bromothiazole-4-carboxamido)octanoic acid (S18)

Synthesized according to general hydrolysis conditions. 83% yield. ¹¹H NMR (600 MHz, DMSO) δ 8.50 (t, J=5.9 Hz, 1H), 8.23 (s, 1H), 3.20 (dd, J=13.6, 6.7 Hz, 2H), 2.17 (t, J=7.4 Hz, 2H), 1.50-1.43 (m, 4H), 1.25 (s, J=2.1 Hz, 6H). ¹³C NMR (151 MHz, DMSO) δ 175.06, 159.64, 150.33, 136.69, 128.74, 39.20, 34.11, 29.48, 28.95, 28.90, 26.71, 24.91.

### 2-Bromo-N-(8-oxo-8-0(tetrahydro-2H-pyran-2-yl)oxy)amino)octyl)thiazole-4-carboxamide (S19)

Synthesized according to the general NH₂ OTHP coupling conditions. 72% yield. ¹¹H NMR (600 MHz, CDCl₃) δ 8.88 (s, 1H), 8.08 (t, J=15.9 Hz, 1H), 7.26 (s, J=14.1 Hz, 1H), 4.94 (s, 1H), 3.94 (s, 1H), 3.62-3.59 (m, 1H), 3.44-3.38 (m, 2H), 2.13 (m, 2H), 1.85-1.76 (m, 4H), 1.66-1.45 (m, 12H). ¹³C NMR (151 MHz, CDCl₃) δ 170.67, 159.84, 149.97, 135.86, 102.52, 62.60, 39.21, 38.82, 29.39, 28.52, 28.03, 27.79, 24.93, 18.66.

### 2-Ethynyl-N-(8-oxo-8-0(tetrahydro-2H-pyran-2-yl)oxy)amino)octyl)thiazole-4-carboxamide (S20)

Synthesized according to general Sonogashira coupling conditions. 34% yield. ¹¹H NMR (600 MHz, CDCl₃) δ 8.70 (s, 1H), 8.16 (s, 1H), 7.34 (s, 1H), 4.95 (s, 1H), 3.95 (t, J=9.0 Hz, 1H), 3.61 (ddd, J=9.8, 4.8, 3.5 Hz, 1H), 3.54 (s, 1H), 3.43 (dd, J=13.4, 6.8 Hz, 2H), 2.10 (s, 2H), 1.84-1.54 (m, 10H), 1.34 (s, 6H). ¹³C NMR (151 MHz, CDCl₃) δ 160.31, 150.65, 147.23, 125.11, 102.52, 83.08, 75.70, 62.61, 39.13, 33.08, 29.42, 28.65, 28.35, 28.03, 26.37, 25.03, 18.66.

### 2-Ethynyl-N-(8-(hydroxyamino)-8-oxooctyl)thiazole-4-carboxamide (S21)

Compound (S21) was synthesized using the general THP deprotection conditions. 62% yield. ¹¹H NMR (600 MHz, acetone-d₆) δ 10.07 (s, 1H), 8.40 (s, 1H), 8.29 (s, 1H), 7.96 (s, 1H), 4.43 (s, 1H), 3.43 (dd, J=13.4, 6.8 Hz, 2H), 2.11 (dd, J=9.4, 5.3 Hz, 2H), 1.67-1.56 (m, 4H), 1.42-1.25 (m, 6H). ¹³C NMR (151 MHz, acetone-d₆) δ 169.98, 159.77, 151.29, 147.13, 125.27, 84.25, 75.70, 38.87, 38.74, 32.24, 29.47, 28.77, 28.67, 26.54, 25.21.

### (Z)-8-(2-bromothiazol-4-yl)oct-7-enoic acid (S25)

Synthesized according to general hydrolysis conditions. (0.27 mmol scale, 92% yield.) ¹H NMR (600 MHz, acetone-d 6) δ 10.47 (br, 1H), 7.46 (s, 1H), 6.38 (dt, J=11.7, 1.5 Hz, 1H), 5.81-5.75 (m, 1H), 2.65-2.60 (m, 2H), 2.33-2.28 (m, 2H), 1.68-1.61 (m, 2H), 1.55-1.49 (m, 2H), 1.46-1.40 (m, 2H). ¹³C NMR (151 MHz, acetone-d 6) δ 173.67, 153.95, 134.90, 134.24, 120.92, 33.24, 29.02, 28.64, 28.57, 24.62.

### (Z)-8-(2-bromothiazol-4-yl)-N-((tetrahydro-2H-pyran-2-yl)oxy)oct-7-enamide (S26)

Synthesized according to the general NH₂ OTHP coupling conditions (0.9 mmol scale, 90% yield). ¹H NMR (600 MHz, CDCl₃) δ 8.53 (s, 1H), 7.02 (s, 1H), 6.36 (d, J=11.7 Hz, 1H), 5.75 (dt, J=11.8, 7.3 Hz, 1H), 4.96 (s, 1H), 3.95 (d, J=8.8 Hz, 1H), 3.63 (dtd, J=11.2, 4.2, 1.6 Hz, 2H), 2.52 (q, J=6.8 Hz, 3H), 2.20-2.02 (m, 2H), 1.87-1.76 (m, 4H), 1.73-1.56 (m, 9H), 1.50 (dt, J=14.5, 7.2 Hz, 4H), 1.42 (dt, J=13.1, 6.5 Hz, 4H). ¹³C NMR (151 MHz, CDCl₃) δ 170.58, 153.82, 135.50, 134.75, 121.17, 119.43, 102.53, 62.58, 33.32, 29.09, 28.76, 28.00, 25.19, 25.02, 18.60.

### (Z)-8-(2-ethynylthiazol-4-yl)-N-((tetrahydro-2H-pyran-2-yl)oxy)oct-7-enamide (S27)

Synthesized according to general Sonogashira coupling conditions (0.06 mmol scale, 65% yield). ¹H NMR (600 MHz, CDCl₃) δ 8.38 (s, 1H), 7.12 (s, 1H), 6.44 (d, J=11.7 Hz, 1H), 5.80 (dt, J=11.7, 7.3 Hz, 1H), 4.96 (s, 1H), 3.98-3.93 (m, 1H), 3.66-3.62 (m, 1H), 2.55 (q, J=6.9 Hz, 2H), 2.16-2.11 (m, 2H), 1.87-1.77 (m, 2H), 1.73-1.58 (m, 6H), 1.51 (dd, J=14.7, 7.4 Hz, 2H), 1.42 (dd, J=14.6, 7.5 Hz, 2H). ¹³C NMR (151 MHz, CDCl₃) δ 170.59, 154.17, 146.24, 135.65, 121.49, 117.99, 102.52, 81.88, 76.69, 62.56, 33.33, 29.04, 28.88, 28.75, 27.99, 25.16, 25.01, 18.58.

### (E)-8-(2-bromothiazol-4-yl)oct-7-enoic acid (S31)

Synthesized according to general hydrolysis conditions. (1.3 mmol scale, 92% yield). 1 H NMR (600 MHz, MeOH-d₄) δ 7.22 (s, 1H), 6.58-6.51 (m, 1H), 6.38 (dt, J=15.5, 1.3 Hz, 1H), 2.32 (t, J=7.4 Hz, 2H), 2.24 (tt, J=7.0, 3.4 Hz, 2H), 1.65 (dt, J=15.1, 7.5 Hz, 2H), 1.53 (dt, J=14.9, 7.4 Hz, 2H), 1.44 -1.39 (m, 2H). ¹³C NMR (151 MHz, MeOH d₄) δ 176.24, 154.60, 135.80, 134.46, 121.93, 117.04, 33.48, 32.05, 28.47, 28.36, 24.53.

### (E)-8-(2-bromothiazol-4-yl)-N-((tetrahydro-2H-pyran-2-yl)oxy)oct-7-enamide (S32)

Synthesized according to the general NH 2 OTHP coupling conditions (530 mg, 1.27 mmol, 90%). ¹H NMR (600 MHz, CDCl₃) δ 8.26 (s, 1H), 6.90 (s, 1H), 6.62-6.55 (m, 1H), 6.32 (d, J=15.5 Hz, 1H), 4.96 (s, 1H), 3.99-3.92 (m, 1H), 3.65 (m, 1H), 2.23 (q, J=7.0 Hz, 2H), 2.12 (S, 2H), 1.88-1.80 (m, 4H), 1.71-1.62 (m, 6H), 1.53-1.48 (m, 2H), 1.42-1.38 (m, 2H). ¹³C NMR (151 MHz, CDCl₃) δ 156.74, 154.70, 135.84, 122.10, 116.33, 100.51, 33.35, 32.40, 29.12, 28.60, 27.97, 25.28, 25.01, 20.31, 18.56.

### (E)-8-(2-ethynylthiazol-4-yl)-N-((tetrahydro-2H-pyran-2-yl)oxy)oct-7-enamide (S33)

Synthesized according to general Sonogashira coupling conditions (0.167 mmol scale, 67% yield). ¹¹H NMR (600 MHz, CDCl₃) δ 8.23 (br, J=20.4 Hz, 1H), 7.00 (s, 1H), 6.64 (dt, J=14.4, 7.0 Hz, 1H), 6.39 (d, J=15.6, 1.3 Hz, 1H), 4.96 (s, 1H), 3.95 (t, J=7.5 Hz, 1H), 3.67-3.63 (m, 1H), 2.24 (dd, J=13.9, 6.9 Hz, 2H), 2.12 (s, 2H), 1.87-1.78 (m, 4H), 1.71-1.59 (m, 6H), 1.51 (dt, J=14.7, 7.2 Hz, 2H), 1.44-1.37 (m, 2H). ¹³C NMR (151 MHz, CDCl₃) δ 170.55, 155.01, 147.07, 135.34, 122.35, 114.99, 102.53, 81.95, 76.61, 62.57, 33.38, 32.49, 29.72, 28.66, 27.96, 25.19, 25.01, 18.54.

### Docking

The docking analysis was accomplished using Glide tool on maestro version 10.6. A library of the compounds was generated utilizing the ligand prep tool allowing possible ionization states at pH=7±2. Protein structures were obtained from protein data bank (PDB) and their structures were prepared using the protein preparation wizard. The prepared protein structures were used to generate the receptor grid using the receptor grid generation tool. The active site was determined by utilizing the co-crystalized ligand. Metal coordination to the zinc ion was implemented as a constraint. Rotation of amino acid side chains was allowed only within the determined active site. Then the library of the compounds was screened with the obtained receptors utilizing the ligand docking tool using extra precision and OPLS3 force field. The results were analyzed with the xp visualizer tool and pose analysis was performed with the pose viewer tool. For the pose analysis the Maestro version 13.1 for academic use was used.

### Cell Culture and Cell Viability Assays for NCI-H522, HCT-116, WI38, and RPE

HCT116 human colorectal carcinoma cells, NCI-H522 human lung cancer cells, WI38 diploid human cell line, and human retinal pigment epithelial RPE cells were maintained in Dulbecco's Modified Eagle's medium (Mediatech, Inc.) supplemented with 10% calf serum (Atlanta Biologicals) or 10% fetal bovine serum (Gemini Bio-Products # 100-106) and 1000 U/ml of both Penicillin and Streptomycin (Mediatech, Inc.) at 37° C. with 5% CO₂. Cell viability was assessed using methylene blue staining: cells were plated at 5000/well in 96 well plates (n=3) respectively and treated the next day. Three days after treatment cells were fixed/stained in methylene blue saturated in 50% ethanol for 30 min at RT. Plates were washed with excess water to wash off extra dye. Retained dye was dissolved in 0.1 N HCl and absorbance was measured at 668 nm.

### Cell Culture and Cell Viability Assays for SH-SY5Y Cells

Human neuroblastoma cells (SH-SY5Y) were grown on poly-D-lysine coated plates and cultured in DMEM/F12 medium (HyClone, Thermo Scientific) containing 10% fetal bovine serum and 1% penicillin-streptomycin. Cells were maintained in a 37° C. incubator with a 5% CO₂ atmosphere. Cells were seeded at a density of 15,000 cells in 96 well plates for 36 hr before treatment of drugs.

### MTT Cell Proliferation and Viability Assay

Cell viability was assessed using the MTT reagent 3-[4,5 dimethylthiazol-2-yl]-2,5-diphenyltetrazolium bromide (MTT, Promega Corporation USA, Ref no. G4102) according to the manufacturer's instructions. Cells were treated with amide series analogs for 24 hrs. Briefly, the cells were incubated with MTT reagent for 2 h at 37° C. incubator. Then, the solubilization/stop solution was added to solubilize the formazan products, incubated for 1 hr, and absorbance at 570 was measured using micro-plate reader (SynergyH1, BioTek, USA). DMSO was used a vehicle control. The experimental data represent the means±SD (*P<0.05) of triplicates from three separate experiments. The statistical significance was determined by one-way ANOVA and significant differences P<0.05, P<0.01, P<0.001, and P<0.0001 are symbolized as *, **, ***, and ****, respectively.

### Cytotoxicity Evaluation on Undifferentiated PC-12 Cells

Pheochromocytoma (PC-12) cells were grown on poly-D-lysine coated plates and cultured in DMEM/F12 medium (HyClone, Thermo Scientific) containing 5% fetal bovine serum, 5% horse serum, and 1% penicillin-streptomycin. Cells were maintained in a 37° C. incubator with a 5% CO₂ atmosphere. Cells were seeded at a density of 15,000 cells per well in 96 well plates for 36 hr before treatment with the test compounds. The MTT assay was performed after 24 hr of treatment.

### Cytotoxicity Evaluation on Differentiated PC-12 Cells

Pheochromocytoma (PC-12) cells were seeded on poly-D-lysine coated plates containing neuron differentiation medium (100 ng nerve growth factor, DMEM/F12, 5% fetal bovine serum, 5% horse serum, and 1% penicillin-streptomycin) and grown for 5 days to differentiate to neurons. Cells were maintained in a 37° C. incubator with a 5% CO₂ atmosphere. Cells were trypsinized, and seeded at a density of 15,000 cells/well in 96 well plates with neuron differentiation medium for 36 hr before treatment with the test compounds. The MTT assay was performed after 24 hr of treatment.

### Microscopy

For time lapse imaging, NCI-H522 cells were plated at ˜70% density and left overnight to adhere and pre-equilibrate to 10% CO₂. The next day cells were treated with DMSO or inhibitors, and the flask was maintained sealed. Flasks were placed on a 37° C. heated stage on an inverted microscope. Images were captured every 12 min and for a total of 300 images using a ×40 microscope objective and an Olympus C740 digital camera controlled by AmScope software. Image analysis was done using Image J. For the Kaplan-Maier graphs at least 100 cells were counted and the event of cell death was measured on a time frame of either 1 h or 0.2 h.

### Flow Cytometry

### C11-BODIPY Lipid Peroxidation Assay

NCI-H522 cells (˜70% density) were plated on 7 cm dishes and let overnight to adhere. The next day cells were treated with DMSO, Inhibitors (10 mM) [with (0.25 mM) or without Liproxstatin-1]. Bodipy 581/591 C11 (1 μM) (ThermoFischer) was added at the time of treatment. After 6 h, cells were washed with 1×PBS and collected by trypsinization and centrifugation. The cells were washed once with 1×PBS and resuspended in PBS containing 2% FBS. Cells were analyzed using a BD LSR Fortessa FACScanner and FlowJo software. For each sample 20×10³ cells were analyzed.

### Cell Cycle Analysis

NCI-H522 cells (5*10⁵ cells per condition) were plated on 7 cm dishes and left overnight to adhere. After 12 h, 24 h, or 48 h of treatment with DMSO or inhibitors (in presence of Liproxstatin-1 0.25 mM), floating and attached cells were collected by trypsinization and centrifugation. Cells were then washed once with 1×PBS and fixed by pre-chilled ethanol (70% final concentration). After fixation, cells were collected by centrifugation and resuspended in PBS, followed by treatment with RNase H (10 μg/ml) for 30 min at 37° C. Cells again were collected by centrifugation and resuspended in PBS containing 2% FBS and propidium iodide. Cells were analyzed using a BD LSR Fortessa FACScanner and FlowJo software. For each sample 20×10³ cells were analyzed.

### Western Blotting

NCI-H522 cell pellets obtained after the corresponding treatment conditions were lysed using lysis buffer containing 50 mM Tris (pH 7.4), 150 mM NaCl, 0.5% NP-40, (supplemented with 1 μg/ml aprotinin, 2 μg/ml leupeptin, 1 μg/ml pepstatin A, 1 mM DTT, and 0.1 M PMSF) for 30 minutes on ice and centrifuged at 13×10³ g for 25 minutes at 4° C. The protein levels of the obtained lysates were normalized using BCA Protein Assay Kit (Pierce) and separated by SDS-polyacrylamide gel electrophoresis (12.5% acrylamide). Transfer to polyvinylidene difluoride membranes (Millipore) was followed by blocking of membranes with blocking buffer containing 5% (w/v) non-fat dry milk dissolved in PBST [1×PBS containing 0.05% (v/v) Tween 20] for 1 hour at room temperature. Membranes were then incubated with corresponding primary antibodies overnight at 4° C. The membranes were then washed (3×15 min each) with PBST and incubated with secondary antibodies conjugated to horse-radish peroxidase, obtained from Biorad and used at a dilution of 1:10,000. Bound antibodies were detected using enhanced chemiluminescence (Biorad).

### Isoform Selectivity

Inhibitor testing with HDAC class I, IIa, and IIb: In a half-area 96-well white opaque plate (Corning), recombinant HDAC1 (1 μL; 3 ng/μL, BPS Bioscience), HDAC2 (1 μL; 1 ng/μL, BPS Bioscience), HDAC3 (1 μL; 30 ng/μL, BPS Bioscience), HDAC6 (1 μL; 35 ng/μL, BPS Bioscience), and HDAC8 (1 μL; 70 ng/μL, BPS Bioscience) were added to HDAC-Glo™ buffer (43 μL) provided by the manufacturer (Promega). In the case of HDAC4, recombinant HDAC4 (1 μL; 2 ng/μL, BPS Bioscience) was added to HDAC-Glo IIa™ buffer (43 μL) provided by Promega. For negative controls, either the HDAC-Glo™ or the HDAC-Glo™ IIa buffer (44 μL) alone was used. Serial dilutions or single concentrations of inhibitors (1 μL in DMSO, concentrations shown in Tables S4-9) or DMSO alone (1 μL) were added to the enzyme solution, followed by 3 hr. incubation at room temperature. For HDAC1, 2, 3, 6, and 8, the HDAC-Glo™ reagent was prepared using the pre-measured lyophilized HDAC-Glo I/II Substrate (Promega) and dissolving in the buffer provided (10 mL). To activate the HDAC-Glo I/II Substrate, the developer reagent was added (1 μL for every 1 mL of HDAC-Glo I/II Substrate solution). For HDAC4, the HDAC-Glo IIa™ reagent was prepared using the pre-measured lyophilized HDAC-Glo Ha luciferase (Promega) and dissolving in the HDAC-Glo IIa buffer provided (10 mL). To activate, the HDAC-Glo IIa substrate (7 μL for every 1 mL of buffer luciferase solution) was added to the luciferase buffer solution and incubated at 37° C. for 1 hour. The developer reagent was added (1 μL for every 1 mL of HDAC-Glo IIa substrate/luciferase solution). The HDAC-Glo™ reagent (5 μL) was added to each reaction containing HDAC1, 2, 3, 5, and 8, whereas the HDAC-Glo™ IIa reagent (5 μL) was added to each reactions containing HDAC4. Luminescent signal was measured every 3 minutes over the course of 30 minutes using an M-Plex Infinite 200 Pro (Tecan). To determine IC₅₀, the luminescent signal at peak reading was first background corrected with the signal from a background control reaction where HDAC was excluded. The background corrected luminescence signal of each inhibitor-containing reaction was divided by the signal of the reaction without inhibitor for each HDAC enzyme to generate a percent deacetylase activity remaining value. IC₅₀ values were calculated by fitting the percent deacetylase activity remaining as a function of inhibitor concentration to a sigmoidal dose-response curve (y=100/(1+(x/IC₅₀)^{z}), where y=percent deacetylase activity and x=inhibitor concentration) using non-linear regression with KaleidaGraph 4.1.3 software.

### Cinnamate Analogs

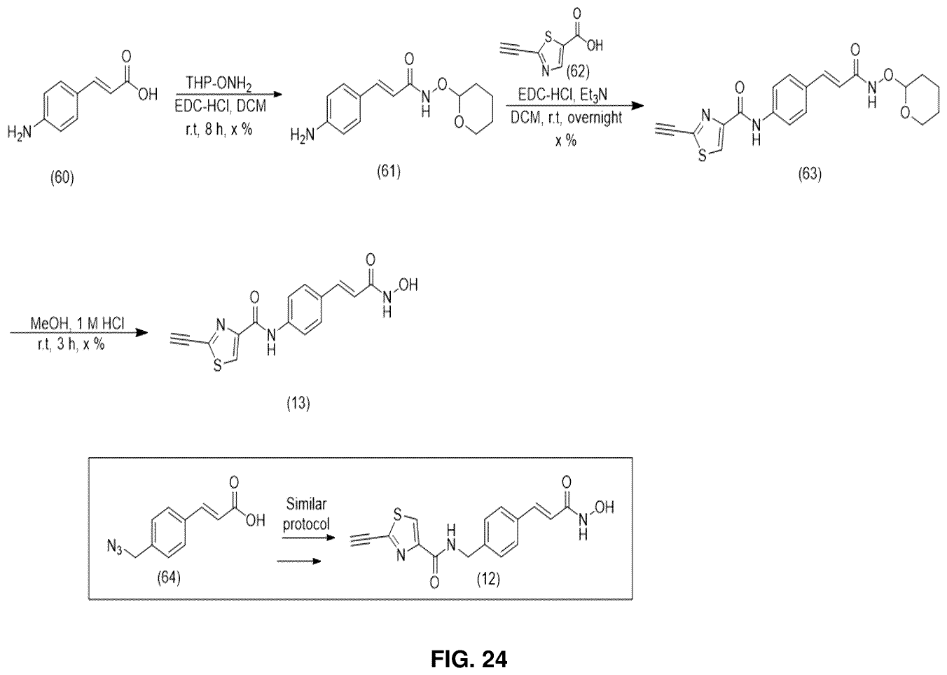

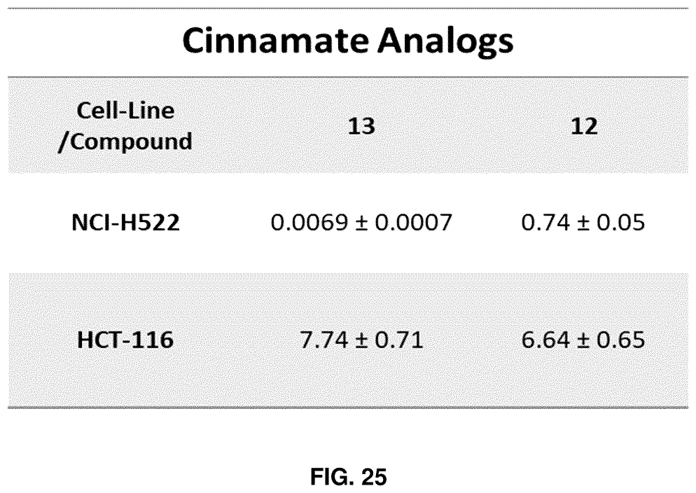

FIG. 24 depicts the synthesis of cinnamate analogs. The cinnamate analogs were tested on NCI-H522 and HCT-116 cell lines (FIG. 25), and showed low cytotoxicity on HCT-116 cells, indicating insufficient HDAC inhibition. H522 cells were more sensitive to the cinnamate analogs, especially to hybrid molecule 12 (IC₅₀ of 6.9 nM).

### Abbreviations Used

BDR, bromodomain; CD, Catalytic Domain; CDK, cyclin dependent kinases; CNS, central nervous system; d, doublet (spectral); DCM, dichloromethane; EMT, epithelial to mesenchymal transition; ESI, electrospray ionization; FC, ferroptosis control; FDA, Food and Drug Administration; GPX4, glutathione peroxidase 4; HAT, histone acetyl transferase; HC, HDAC control; HDAC, histone deacetylase; HDACi, histone deacetylase inhibitor; HPLC, high performance liquid chromatography; HRMS, high-resolution mass spectra; HY, hybrid molecules ; MEF2, myocyte enhancer factor-2; NAD, nicotinamide adenine dinucleotide; NC, negative control; NCI, National Cancer Institute; NMR, nuclear magnetic resonance; PDB, protein data bank ; PI, propidium iodide; PUFAs, polyunsaturated fatty acids; q, quartet (spectral); ROS, reactive oxygen species; RPE, retinal pigment epithelial; RPE, retinal pigment epithelial; s, singlet (spectral); SAR, structure activity relationship; SD, standard deviation; t, triplet (spectral); THP, tetrahydropyran; THP, tetrahydropyran-2-yl; TLC, thin layer chromatography; TMS, trimethylsilyl; TNBC, triple-negative breast cancer; Topo, topoisomerase ; UV, ultraviolet; ZBG, zinc binding group; Z-VAD-FMK, N-benzyloxycarbonyl-Val-Ala-Asp(O-Me) fluoromethyl ketone.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A composition comprising a hybrid molecule having both a ferroptotic pharmacophore and a histone deacetylase (HDAC) inhibitor pharmacophore.

2. The composition of claim 1, wherein the ferroptotic pharmacophore comprises a terminal alkyne attached to a thiazole.

3. The composition of claim 1, wherein the HDAC inhibitor pharmacophore comprises a hydroxamic acid metal-binding group.

4. The composition of claim 1, wherein the hybrid molecule has Formula A: 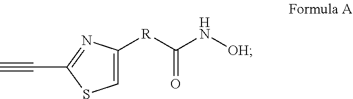
wherein:
R is alkyl, alkenyl, amidoalkyl, amidoalkenyl, arylalkyl, arylalkenyl, or amidoarylalkenyl;
and salts, stereoisomers, racemates, solvates, hydrates, and polymorphs thereof.

5. The composition of claim 4, wherein R is (C1-C6)alkyl or (C1-C6)alkenyl.

6. The composition of claim 1, wherein the hybrid molecule is HY-1:

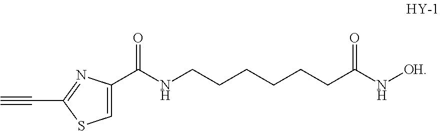 [SMILES: C#Cc1nc(C(=O)NCCCCCCC(=O)NO)cs1]

7. The composition of claim 1, wherein the hybrid molecule is HY-2:

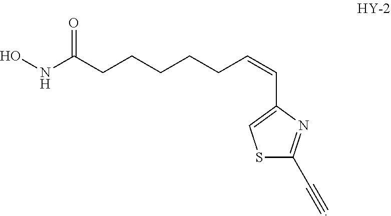

8. The composition of claim 1, wherein the hybrid molecule is HY-3:

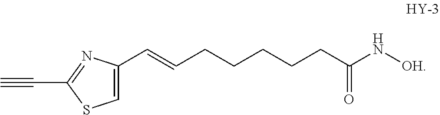 [SMILES: C#Cc1nc(/C=C/CCCCCC(=O)NO)cs1]

9. The composition of claim 1, wherein the hybrid molecule is HY-4:

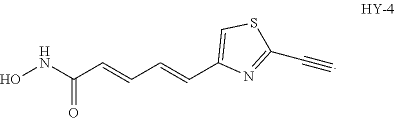

10. The composition of claim 1, wherein the hybrid molecule is HY-5:

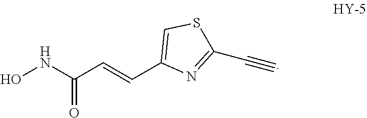

11. A composition comprising a histone deacetylase (HDAC) inhibitor having Formula C: 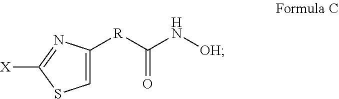
wherein:
X is a halide, and
R is an alkyl, alkenyl, alkynyl, amidoalkyl, amidoalkenyl, arylalkyl, arylalkenyl, or amidoarylalkenyl group;
and salts, stereoisomers, racemates, solvates, hydrates, and polymorphs thereof.

12. The composition of claim 11, wherein X is Br, and R includes an aliphatic chain of from 4 to 6 carbons.

13. The composition of claim 11, wherein X is Br, and R includes an alkenyl group.

14. The composition of claim 11, wherein the HDAC inhibitor is HC-1:

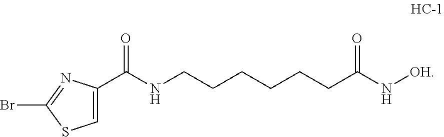 [SMILES: O=C(CCCCCCNC(=O)c1csc(Br)n1)NO]

15. The composition of claim 11, wherein the HDAC inhibitor is HC-2:

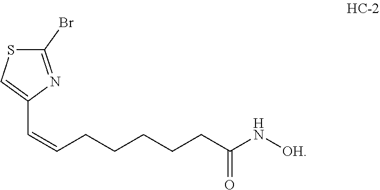 [SMILES: O=C(CCCCC/C=C\c1csc(Br)n1)NO]

16. The composition of claim 11, wherein the HDAC inhibitor is HC-3:

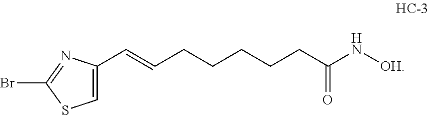 [SMILES: O=C(CCCCC/C=C/c1csc(Br)n1)NO]

17. The composition of claim 11, wherein the HDAC inhibitor is HC-4:

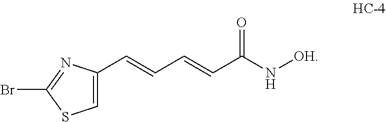 [SMILES: O=C(/C=C/C=C/c1csc(Br)n1)NO]

18. The composition of claim 11, wherein the HDAC inhibitor is HC-5:

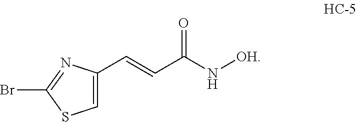 [SMILES: O=C(/C=C/c1csc(Br)n1)NO]

19. A method of killing cancer cells, the method comprising contacting cancer cells with an effective amount of a composition comprising Formula A, Formula B, or Formula C, and killing the cancer cells: 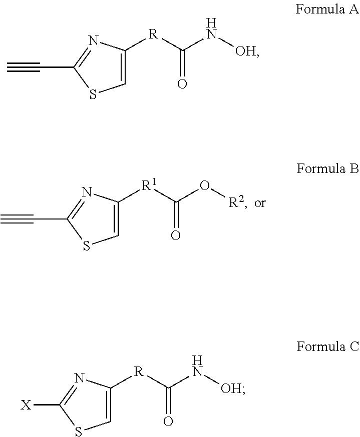
wherein:
R is alkyl, alkenyl, amidoalkyl, amidoalkenyl, arylalkyl, arylalkenyl, or amidoarylalkenyl;
X is a halide;
R¹ is alkyl, alkenyl, amidoalkyl, amidoalkenyl, arylalkyl, arylalkenyl, or amidoarylalkenyl; and
R² is alkyl.

20. The method of claim 19, wherein the cancer is triple negative breast cancer, renal cancer, leukemia, colon cancer, ovarian cancer, breast cancer, lung cancer, melanoma, or CNS cancer.

21. A method of treating a cancer, the method comprising administering to a subject having cancer an effective amount of a composition comprising Formula A or Formula C, and treating the cancer: 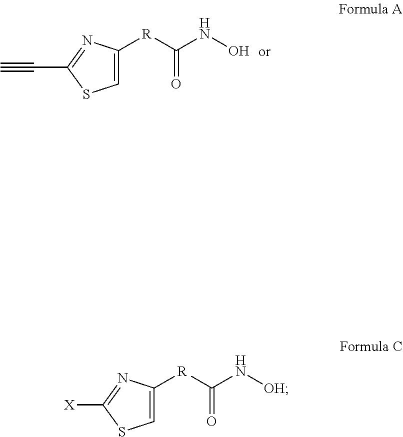
wherein:
R is alkyl, alkenyl, amidoalkyl, amidoalkenyl, arylalkyl, arylalkenyl, or amidoarylalkenyl; and
X is a halide.

22. A method of inhibiting histone deacetylase (HDAC) in a subject, the method comprising administering to the subject an effective amount of a composition comprising Formula A or Formula C, and inhibiting HDAC in the subject: 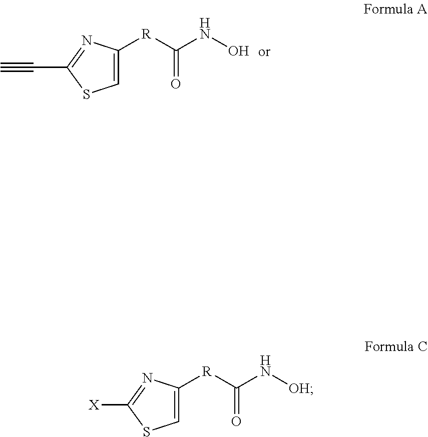
wherein:
R is alkyl, alkenyl, amidoalkyl, amidoalkenyl, arylalkyl, arylalkenyl, or amidoarylalkenyl; and
X is a halide.
